(19) United States
(12) Patent Application Publication
(10) Pub. No.: US 2022/0193275 A1
(43) Pub. Date: Jun. 23, 2022

(54) INDUCING FAVORABLE EFFECTS ON TUMOR MICROENVIRONMENT VIA ADMINISTRATION OF NANOPARTICLE COMPOSITIONS

(71) Applicants: Memorial Sloan Kettering Cancer Center, New York, NY (US); Cornell University, Ithaca, NY (US)

(72) Inventors: Michelle S. Bradbury, New York, NY (US); Steven Saeid Zanganeh, New York, NY (US); Brian Madajewski, New York, NY (US); Luis Felipe Campesato, New York, NY (US); Taha Marghoub, Jersey City, NJ (US); Michael Overholtzer, Glen Ridge, NJ (US); Michael R. McDevitt, New York, NY (US); Ulrich Wiesner, Ithaca, NY (US)

(21) Appl. No.: 17/414,414

(22) PCT Filed: Dec. 17, 2019
(86) PCT No.: PCT/US19/66944

§ 371 Date: Jun. 16, 2021

Related U.S. Application Data
(60) Provisional application No. 62/780,791, filed on Dec. 17, 2018.

(51) Int. Cl. A61K 51/12; A61K 9/16; A61K 51/08; A61K 51/04; A61K 38/20; A61K 38/21; A61K 31/499; A61K 31/245; A61K 39/395; A61K 9/50; A61P 35/00

(52) U.S. Cl. CPC A61K 51/1244; A61K 9/1611; A61K 51/088; A61K 51/0482; A61K 51/0478; A61P 35/00; A61K 38/217; A61K 31/499; A61K 31/245; A61K 39/3955; A61K 9/5031; A61K 38/2013

(57) ABSTRACT

Described herein are methods of treating cancer by inducing favorable effects on tumor microenvironment (e.g., including macrophage polarization, cytokine profile, and/or immunophenotype) via administration of nanoparticles (e.g., silica-based ultra-small nanoparticles and nanoparticle conjugates such as nanoparticle drug conjugates). In certain embodiments, the methods may be used in concert with, or as part of, checkpoint inhibition therapy (e.g., anti-PD1) or radiotherapy, or a combination of both radiotherapy and checkpoint inhibitor therapy.

### CROSS REFERENCE TO RELATED APPLICATIONS

This application claims the benefit of U.S. Application Ser. No. 62/780791 filed on Dec. 17, 2018, the disclosure of which is hereby incorporated by reference in its entirety.

### GOVERNMENT SUPPORT

This invention was made with government support under grant numbers CA132378, CA008748, CA161280, and CA55349 awarded by the National Institutes of Health. The government has certain rights in the invention.

### FIELD OF THE INVENTION

This invention relates generally to methods and compositions for the treatment of cancer in subjects. More specifically, in certain embodiments, the invention relates to methods of treating cancer by inducing favorable effects on tumor microenvironment (e.g., including macrophage polarization, cytokine profile, and/or immunophenotype) via administration of nanoparticles (e.g., silica-based nanoparticles and nanoparticle conjugates such as nanoparticle drug conjugates).

### BACKGROUND OF THE INVENTION

Identifying new therapies that can eliminate cancer has been a significant research and clinical goal for decades. Cancer cells have traditionally been targeted with pharmacological agents that are either preferentially cytotoxic to dividing cells, or that block specific cancer-activated pathways to inhibit division or induce cell death. Such treatments, which generally induce apoptosis with or without unregulated necrosis, are associated with significant toxic effects on normal tissues or fail to eliminate all cells within cancerous lesions, limiting efficacy and promoting tumor recurrence. While immune checkpoint blockade and engineered cellular therapies (e.g., chimeric antigen receptor (CAR) T cells) have yielded dramatic responses in hard-to-treat tumors, their use is limited by ineffective solid tumor tissue penetration, off-target effects in immunosuppressed tumor microenvironments, and/or toxic side-effect profiles.

### SUMMARY OF THE INVENTION

Presented herein are methods and compositions for the treatment of cancer in subjects and for improving the immunogenicity of the tumor microenvironment to overcome limitations of prior technologies. More specifically, in certain embodiments, the invention relates to methods of treating cancer by inducing favorable effects on tumor microenvironment (e.g., including macrophage polarization, cytokine profile, and/or immunophenotype) via administration of nanoparticles (e.g., silica-based nanoparticles and nanoparticle conjugates such as nanoparticle drug conjugates).

Surprisingly, it has been found that administration of even low dosages of nanoparticles (e.g., C′ dots) induces favorable changes in the immune profile of the tumor microenvironment without being cytotoxic to normal tissues. For example, macrophages have been found to be activated or polarized in response to the administration of C′ dots as described herein. Macrophages play an important role in recognition and destruction of cancer cells within the tumor microenvironment. Use of nanoparticles at low dosages in vivo or in vitro enhances response of anti-tumorigenic phenotypes (e.g., M1 phenotype) of macrophages in the microenvironment, and suppresses the activation of anti-inflammatory macrophages (e.g., M2 macrophages) understood to be pro-tumorigenic. These effects occur independently of ferroptotic-induction of cell death in the tumor microenvironment, which is known to occur when higher concentrations of nanoparticles are administered to subjects. Furthermore, following treatment with nanoparticles, the progression of tumors is stalled both in vitro and in vivo. Accordingly, delivery of nanoparticles to tumor microenvironment at lower dosages may be used to augment the immune response of cells in the tumor microenvironment and/or halt tumor progression, while avoiding the negative effects of administering high dosages of drug to a subject.

Moreover, it was also found that nanoparticles targeted to a tumor microenvironment also induce changes in the immune profile and tumor progression of the in vivo tumor microenvironment when conjugated with a radionucleotide. For example, conjugating peptides (e.g., αMSH) to nanoparticles allowed nanoparticles to be targeted to melanoma or glioma tumors in a subject. αMSH-PEG-Cy5 C′ dots, radiolabeled with 225-Actinium, were found to be cytotoxic to tumor cells due to the delivery of radiation to tumor in a targeted manner. Surprisingly, it was found that, both radiolabeled “hot” [²²⁵Ac]αMSH-PEG-Cy5 C′ dots and “cold” αMSH-PEG-Cy5 C′ dots caused changes in the immune profiles of the tumor microenvironment, and the administration of either resulted in a decrease in tumor volume and/or slowed tumor growth. Accordingly, the C′ dot component itself was found to initiate a favorable pseudo-pathogenic response in the tumor microenvironment. Furthermore, this is observed through distinct changes in the fractions of naive and activated CD8 T cells, Th1 and regulatory T cells, immature dendritic cells, monocytes, MΦ and M1 macrophages, and activated natural killer cells. Therefore, the administration of tumor targeting C′ dots is a potent modulator of the microenvironment, and C′ dots may be administered in combination with other therapies such as checkpoint blockade therapy or radiotherapy to enhance the anti-tumorigenic nature of the tumor microenvironment.

In one aspect, in the invention is directed to a method of treatment of a subject (e.g., a subject having been diagnosed with cancer), the method comprising administering a composition comprising ultrasmall (e.g., no greater than 20 nm in diameter, e.g., no greater than 10 nm in diameter) nanoparticles (e.g., a silica-containing, e.g., silica-based nanoparticle) to activate a tumor microenvironment (e.g., macrophages, T cells, and/or antigen-presenting cells (APCs, such as dendritic cells)).

In certain embodiments, the method comprises administering the composition comprising ultrasmall nanoparticles in concert with, or as part of, checkpoint inhibitor therapy (e.g., anti-PD1), or radiotherapy, or a combination of both radiotherapy and checkpoint inhibitor therapy.

In certain embodiments, the nanoparticle comprises a radiolabel (e.g., ²²⁵Actinium).

In certain embodiments, the nanoparticle comprises from 1 to 25 targeting ligands (e.g., 2 to 20 ligands, 5 to 15 ligands, 5 to 10 ligands, or 6-8 ligands). In certain embodiments, the targeting ligand is a targeting ligand for a cellular receptor (e.g., MC1-R, PSMA, etc.). In certain embodiments, the targeting ligand comprises αMSH.

In certain embodiments, the nanoparticle does not comprise a targeting ligand. In certain embodiments, the nanoparticle comprises PEG (e.g., a PEG coating).

In certain embodiments, the nanoparticle comprises a heterogeneous surface characterized by one or more of (i) to (iv) as follows: (i) an unincorporated dye; (ii) variation in a PEG coating (e.g., due to length of PEG chains and/or number of PEG chains per nanoparticle, e.g., said number from about 100 to about 500 chains per nanoparticle); (iii) variation in dye encapsulation (e.g., by PEG); and (iv) number of targeting ligands.

In certain embodiments, the nanoparticle has a hydrodynamic diameter no greater than 10 nm (e.g., wherein the hydrodynamic diameter is in a range from 1 nm to 10 nm).

In certain embodiments, the nanoparticle comprises a silica core. In certain embodiments, the silica core has a diameter less than 10 nm (e.g., less than 9 nm, e.g., less than 8 nm, e.g., less than 7 nm, e.g., less than 6 nm, e.g., within a range from 2.7 nm to 5.8 nm).

In certain embodiments, the nanoparticle comprises a polyethylene glycol (PEG) shell. In certain embodiments, the thickness of the PEG shell is less than 2 nm (e.g., about 1 nm).

In certain embodiments, the nanoparticles have a silica composition such that ferroptosis is not induced (e.g., ferroptosis is switched “off”). In certain embodiments, the nanoparticles are made using a ratio of phosphonate-silane to tetramethyl orthosilicate (TMOS) in a reaction feed at or above 20%.

In certain embodiments, the nanoparticles have a silica composition such that ferroptosis may be induced (e.g., ferroptosis is not switched “off”). In certain embodiments, the nanoparticles are made using a ratio of phosphonate-silane to tetramethyl orthosilicate (TMOS) in a reaction feed in a range from about 0% to about 20%.

In certain embodiments, the nanoparticle comprises a chelator. In certain embodiments, the chelator is selected from the group comprising DOTA-Bz-SCN, 1,4,7,10-tetraazacyclododecane-1,4,7,10-tetraacetic acid (DOTA), and desferoxamine (DFO).

In certain embodiments, the nanoparticle is non-toxic to normal tissue.

In certain embodiments, the nanoparticles are internalized (e.g., phagocytosed) within one or more cell types (e.g., macrophages, tumor cells, THP-1 cells) of the microenvironment. In certain embodiments, the one or more cell types comprise macrophages, cancer cells, and/or THP-1 cells.

In certain embodiments, the tumor is a cancer. In certain embodiments, the cancer is a glioma. In certain embodiments, the cancer is melanoma.

In certain embodiments, local concentration of nanoparticles within the microenvironment of the tumor is in a range from about 0.013 nmol/cm³ to about 86 nmol/cm³ or from about 0.013 nmol/cm³ to about 0.14 nmol/cm3 or from about 8 nmol/cm³ to about 86 nmol/cm³ (e.g., wherein an administered dose (e.g., by IV) has particle concentration from about 100 nM to about 60 μM, or wherein an administered dose has particle concentration less than 150 nM (e.g., less than 100 nM, e.g., less than 50 nM, less than 10 nM, less than 5 nM). For example, tumor size may range from about 0.14 g to about 1.5 g; assuming a single dose of 200 μL of a 100 nM nanoparticle solution, microenvironment concentration may be about 0.013 nmol/cm³ for the 1.5 g tumor to about 0.14 nmol/cm³ for the 0.14 g tumor; assuming a single dose of 200 μL of a 60 μM nanoparticle solution, microenvironment concentration may be about 8 nmol/cm³ for the 1.5 g tumor to about 86 nmol/cm³ for the 0.14 g tumor.

In certain embodiments, the activation of the microenvironment of the tumor comprises a change (e.g., an increase) in at least one M1 macrophage polarization marker. In certain embodiments, the at least one M1 macrophage polarization marker is a member selected from the group consisting of iNOS, TNFα, IL12p70, IL12p40, CD86, and CD8.

In certain embodiments, the activation of the microenvironment of the tumor comprises a change (e.g., a decrease) in at least one M2 macrophage polarization marker. In certain embodiments, the at least one M2 macrophage polarization marker is a member selected from the group consisting of IL-4, IL-10, and IL-13.

In certain embodiments, the activation of the microenvironment of the tumor comprises an increase in at least one M1 macrophage polarization marker and a decrease in at least one M2 macrophage polarization marker.

In certain embodiments, the activation of the tumor microenvironment causes a change (e.g., an increase) in one or more cytokines and/or cytolytic proteins. In certain embodiments, the one or more cytokines and/or cytolytic proteins comprises at least one member selected from the group consisting of IL18, IL12, IFN gamma, TNF, and a Granzyme.

In certain embodiments, the activation of the microenvironment comprises changing (e.g., increasing, decreasing) a population and/or level of activation of one or more cell types within the microenvironment. In certain embodiments, the method comprises increasing the population and/or level of activation of one or more immune-related cell types. In certain embodiments, the one or more immune-related cell types comprise at least one member selected from the group consisting of immature dendritic cells, regulatory T cells, monocytes, M1 macrophages, and natural killer cells.

In certain embodiments, the method comprises decreasing the population and/or level of activation of one or more immune-related cell types. In certain embodiments, the one or more immune-related cell types comprise M2 macrophages and/or MΦ macrophages.

In certain embodiments, the composition is administered in multiple doses (e.g., at fixed intervals, e.g., every 1, 2, 3, 5, or 10 days).

In certain embodiments, the method comprises administering a macromolecule (e.g., a protein). In certain embodiments, the macromolecule is an interleukin (e.g., IL12). In certain embodiments, the macromolecule is an interferon (e.g., IFN gamma).

In certain embodiments, the method comprises activating the tumor microenvironment in the absence of ferroptosis.

In certain embodiments, the method comprises activating the tumor microenvironment in the presence of ferroptosis.

In certain embodiments, the method comprises administering one or more regulators of ferroptosis. In certain embodiments, the regulator of ferroptosis is an inhibitor of ferroptosis. In certain embodiments, the one or more inhibitors of ferroptosis comprises a member selected from the group consisting of liproxstatin-1, ferrostatin-1, and/or other compounds which scavenge lipid peroxides.

In another aspect, the invention is directed to a composition for use in the method of any one of the preceding claims, the composition comprising ultrasmall nanoparticles having the following attributes: (i) a number of targeting ligands (e.g., αMSH) from 5 to 15 per nanoparticle; (ii) a heterogeneous surface characterized by one or more of (a) to (d) as follows: (a) an unincorporated dye; (b) a variation in a PEG coating (e.g., due to length of PEG chains and/or number of PEG chains per nanoparticle, e.g., said number from about 100 to about 500 chains per nanoparticle); (c) a variation in dye encapsulation (e.g., by PEG); and (d) a number of targeting ligands (e.g., from 1 to 60 per nanoparticle, or from 1 to 15 per nanoparticle, or from 40 to 60 per nanoparticle); (iii) a particle core and shell having a hydrodynamic diameter in a range from 4.7 nm to 7.8 nm (e.g., with a silica core diameter in a range from 2.7 nm to 5.8 nm and/or with a PEG shell thickness of about 1 nm); and (iv) a silica composition controlled for ferroptosis “switch-off” (e.g., wherein the nanoparticles are made using a ratio of phosphonate-silane to tetramethyl orthosilicate (TMOS) in a reaction feed at or above 20% such that ferroptosis may occur, or wherein the nanoparticles are made using a ratio of phosphonate-silane to tetramethyl orthosilicate (TMOS) in a reaction feed from 0% to 20% such that ferroptosis may not occur.

In another aspect, the invention is directed to a composition (e.g., a pharmaceutical composition) for use in a medicament, the composition comprising ultrasmall nanoparticles having the following attributes: (i) a number of targeting ligands (e.g., αMSH) from 5 to 15 per nanoparticle (ii) a heterogeneous surface characterized by one or more of (a) to (d) as follows: (a) an unincorporated dye; (b) a variation in a PEG coating (e.g., due to length of PEG chains and/or number of PEG chains per nanoparticle, e.g., said number from about 100 to about 500 chains per nanoparticle); (c) a variation in dye encapsulation (e.g., by PEG); and (d) a number of targeting ligands (e.g., from 1 to 60 per nanoparticle, or from 1 to 15 per nanoparticle, or from 40 to 60 per nanoparticle); (iii) a particle core and shell having a hydrodynamic diameter in a range from 4.7 nm to 7.8 nm (e.g., with a silica core diameter in a range from 2.7 nm to 5.8 nm and/or with a PEG shell thickness of about 1 nm); and (iv) a silica composition controlled for ferroptosis “switch-off” (e.g., wherein the nanoparticles are made using a ratio of phosphonate-silane to tetramethyl orthosilicate (TMOS) in a reaction feed at or above 20% such that ferroptosis may occur, or wherein the nanoparticles are made using a ratio of phosphonate-silane to tetramethyl orthosilicate (TMOS) in a reaction feed from 0% to 20% such that ferroptosis may not occur.

In another aspect, the invention is directed to a treatment comprising a therapeutically effective amount of a composition (e.g., wherein the composition comprises a tumor microenvironment activating nanoparticle with a ligand for targeting MC1-R) (e.g., a composition as described herein) for use in a method of treating cancer in a subject.

In another aspect, the invention is directed to a method of treating cancer in a subject, the method comprising: administering a composition (e.g., via IV) to the subject to activate a tumor microenvironment. In certain embodiments, the composition comprises a nanoparticle.

### BRIEF DESCRIPTION OF THE FIGURES

The foregoing and other objects, aspects, features, and advantages of the present disclosure will become more apparent and better understood by referring to the following description taken in conjunction with the accompanying drawings, in which:

FIG. 1A shows a graph of normalized tumor volume over a period of 9 days in mice administered three doses of either αMSH-C′ dots (n=4) or saline vehicle (n=4) at days 0, day 2, and day 6.

FIG. 1B shows histological sections of B16-F10 xenografted tumors in mice having been treated with either saline (top row) or αMSH-C′ dots (bottom row).

FIG. 1C shows a series of graphs indicating the amount of area or number of cells in the tumor microenvironment having tested positive for a marker after treatment with either saline vehicle (‘C’) or αMSH-C′ dots (αMSH C′ dot).

FIGS. 2A-H show graphs of gene expression profiles of mouse bone marrow-derived macrophages (BMDMs) treated with 5 nM C′ dots or 100 nM C′ dots at time points of 24 hours, 1 week (1W), or 2 weeks (2W) in vitro.

FIG. 2A shows a graph of iNOS gene expression profiles of BMDMs.

FIG. 2B shows a graph of TNFα gene expression profiles of BMDMs.

FIG. 2C shows a graph of IL12p70 gene expression profiles of BMDMs.

FIG. 2D shows a graph of IL12p40 gene expression profiles of BMDMs.

FIG. 2E shows a graph of CD86 gene expression profiles of BMDMs.

FIG. 2F shows a graph of Arg1 gene expression profiles of BMDMs.

FIG. 2G shows a graph of CD206 gene expression profiles of BMDMs.

FIG. 2H shows a graph of IL10 gene expression profiles of BMDMs.

FIG. 3A shows immunofluorescent images of BMDMs.

FIG. 3B is a graph of the change in expression of iNOS and CD206 as measured with qRT-PCR in BMDMs.

FIG. 3C is a heat map of M1 and M2 associated polarization markers.

FIG. 3D shows two panels of DIC microscopy images of BMDMs.

FIG. 3E shows a graph of percentage cell survival of BMDMs.

FIG. 4 shows a western blot of BMDMs that were either exposed to either 0, 10 nM, or 100 nM αMSH-C′ dots with or without DFO. At the conclusion of the experiment, cells were collected and expression levels of FTH1 and tubulin were evaluated using western blot.

FIGS. 5A-F show graphs of cytokine release profiles in BMDMs exposed in vitro to either 5 nM PEG-C′ dots or 100 nM PEG-C′ dots for time periods of 6 hours, 24 hours, 48 hours, 1 week, or 2 weeks. CTRL indicates control BMDMs, which were left untreated.

FIG. 5A shows a graph of expression of TNFα in BMDMs.

FIG. 5B shows a graph of expression of IL-12p40 in BMDMs.

FIG. 5C shows a graph of expression of IL-12p70 in BMDMs.

FIG. 5D shows a graph of expression of IL-4 in BMDMs.

FIG. 5E shows a graph of expression of IL-10 in BMDMs.

FIG. 5F shows a graph of expression of IL-13 in BMDMs.

FIG. 6A shows the cytokine expression profile of BMDMs left untreated, treated with 50 nM PEG-C′ dots, or treated with 100 nM PEG-C′ dots at time points of 6hrs, 24 h, and 48 h after initiation of the experiment.

FIG. 6B shows a heat map of gene expression of BMDMs left untreated, treated with 50 nM PEG-C′ dots, or treated with 100 nM PEG-C′ dots at time points of 6hrs, 24 h, and 48 h after initiation of the experiment.

FIG. 6C shows the cytokine expression profile of BMDMs left untreated, treated with 50 nM PEG-C′ dots, or treated with 100 nM αMSH-C′ dots at time points of 6hrs, 24 h, and 48 h after initiation of the experiment.

FIG. 6D shows a heat map of gene expression of BMDMs left untreated, treated with 50 nM αMSH-C′ dots, or treated with 100 nM PEG-C′ dots at time points of 6hrs, 24 h, and 48 h after initiation of the experiment.

FIG. 7A shows a representative flow cytometry plot for BMDM polarization using markers for CD80 and CD206.

FIG. 7B shows the mean fluorescent intensity (MFI) of M1 (CD80) and M2 (CD206) phenotype markers.

FIG. 7C is a representative flow cytometry plot of cell populations labeled with markers CFSE and F4/80.

FIG. 7D is a graph showing the percent of BMDMs having phagocytosed tumor (GBM) cells.

FIG. 8A shows a schematic of an experimental protocol for studying C′ dot administration in a PDGF-B-driven genetically-engineered mouse model of glioblastoma.

FIG. 8B shows a graph of normalized glioblastoma tumor volume over time in the brains of mice treated with αMSH-C′ dots (n=3; square) or only saline vehicle (n=5; circle).

FIG. 8C shows corresponding coronal MR images comparing tumor growth in a mouse administered saline vehicle (top row) and in a mouse administered αMSH-C′ dots (bottom row) at day 0 and day 9 of the experimental procedure as outlined in FIG. 8A.

FIG. 8D shows a H&E (hematoxylin and eosin) stained of brain of a mouse with a tumor outlined in a dashed line. A mouse that was administered only saline vehicle (top panel) is compared with the brain of a mouse having been administered αMSH-C′ dots (bottom panel).

FIG. 8E is a graph indicating the percentage of cells in the tumor microenvironment of a murine brain having been determined to be positive for both Iba1 and CD206 (left panel) or at least Iba1 (right panel) using immunofluorescence.

FIG. 8F shows representative immunofluorescent images from brain tumors and contralateral normal brain from mice that have been administered either αMSH-C′ dots (bottom row of panels) or saline vehicle (top row of panels). Contralateral normal brain images have no tumors.

FIG. 9A shows a schematic of an experimental protocol for studying C′ dot administration in a PDGFB-driven genetically-engineered mouse model of high grade glioblastoma used for conducting the experiment of FIGS. 9B-C.

FIG. 9B shows graphs of flow cytometry studies carried out on cells of brain specimens of mice.

FIG. 9C shows three graphs of the percentage of infiltrating macrophages (left panel), M1-like macrophages (center panel), M2-like macrophages (right panel) from the specimens analyzed in the flow cytometry experiment of FIG. 9B.

FIG. 9D shows a plot obtained from a flow cytometry study where cells were marked to determine the presence of Ki67 and CD45.

FIG. 9E shows a graph of the percentage of non-myeloid cell populations.

FIG. 10A is an illustrative schematic of three tissue sources tested using genetic profiling.

FIG. 10B are graphs of gene profiles for each gene of interest as noted for control (CTRL, no tumor), tumor (i.e., untreated tumor), and tumor having been treated with αMSH-C′ dots.

FIG. 11 shows graphs of secreted cytokines in brains of mice without tumor (WT CTRL brain), untreated tumor (CTRL Tumor-C), and PEG C′ dot treated tumor after 96h.

FIG. 12 show a representative histological image of a mouse brain illustrating regions from which samples are taken for cytokine release studies.

FIG. 13A is a heat map of a cytokine release profile from tissue from tumor center.

FIG. 13B is a heat map of a cytokine release profile from tissue from the tumor boundary.

FIG. 13C is a heat map of a cytokine release profile from brain parenchymal tissue adjacent to and ipsilateral to tumor.

FIG. 13D is a heat map of a cytokine release profile from the tumor center.

FIG. 14A shows graphs of antigen specific T-cell responses to the administration of C′ dots through metrics of T-cell proliferation (left panel) and T-cell activation (right panel).

FIG. 14B shows graphs of antigen-unspecific T-cell responses to the administration of C′ dots through metrics of T-cell proliferation (left panel) and T-cell activation (right panel).

FIG. 15 shows graphs of results of in vitro human dendritic cell activation studies carried out using flow cytometry.

FIG. 16A is a GPC elugram of NH₂-PEG-Cy5-C′ dots.

FIG. 16B is an FCS curves of NH₂-PEG-Cy5-C′ dots with a line fit.

FIG. 16C is a UV-Vis absorbance of NH₂-PEG-Cy5-C′ dots.

FIG. 16D is a GPC elugram of αMSH-PEG-Cy5-C′ dots.

FIG. 16E is an FCS curve of αMSH-PEG-Cy5-C′ dots with a line fit.

FIG. 16F is a UV-Vis absorbance of αMSH-PEG-Cy5-C′ dots.

FIG. 16G is a UV-Vis absorbance spectra of Cy5.

FIG. 16H is a UV-Vis absorbance spectra of αMSH peptide.

FIG. 17A shows an illustrative representation of the molecular structure of [²⁵⁵Ac]αMSH-PEG-Cy5-C′ dots, in an embodiment.

FIG. 17B shows an illustration of the radiosynthesis of [²⁵⁵Ac]αMSH-PEG-Cy5-C′ dots, in an embodiment.

FIG. 17C show an illustrative representation of Actinium-225 decay.

FIG. 18A shows a FACS plot of PDPN-PE-Cy7 versus C′ dot-Cy5 in B16-F10 cells isolated from tumor 4 days after intravenous administration of 50 μmole of αMSH-PEG-Cy5-C′ dots.

FIG. 18B shows a FACS plot of PDPN-PE-Cy7 versus C′ dot-Cy5 in B16-F10 cells isolated from tumor 4 days after intravenous administration of 1% HSA injection.

FIG. 18C shows a FACS plot of F4/80-PE-Cy7 versus C′ dot Cy5 in macrophages isolated from tumor 4 days after intravenous administration of 50 μmole of αMSH-PEG-Cy5-C′ dots.

FIG. 18D shows a FACS plot of F4/80-PE-Cy7 versus C′ dot Cy5 in macrophages isolated from tumor 4 days after intravenous administration of 1% HSA injection.

FIG. 18E shows FACS plot of F4/80-PE-Cy7 versus C′ dot Cy5 in intraperitoneal tissue macrophages harvested from naive mice 2 days after IP administration of 50 μmole of αMSH-PEG-Cy5-C′ dots.

FIG. 18F shows FACS plot of F4/80-PE-Cy7 versus C′ dot Cy5 in intraperitoneal tissue macrophages harvested from naive mice 2 days after IP administration of 1% HSA injection.

FIG. 18G shows a FACS analysis of Forward Scatter (FSC) versus C′ dot Cy5 at 2 days after introduction of 25 μmole of αMSH-PEG-Cy5-C′ dots to B16-F10 cells in vitro.

FIG. 18H shows a FACS analysis of Forward Scatter (FSC) versus C′ dot Cy5 at 2 days after introduction of 1×PBS to B16-F10 cells in vitro.

FIG. 18I shows a FACS analysis of FSC versus C′ dot Cy5 at 2 days after introduction of 25 μmole of αMSH-PEG-Cy5-C′ dots to wild type THP-1 cells in vitro.

FIG. 18J shows a FACS analysis of FSC versus C′ dot Cy5 at 2 days after introduction of 1×PBS to wild type THP-1 cells in vitro.

FIG. 18K shows a FACS analysis of FSC versus C′ dot Cy5 at 2 days after introduction of 25 μmole of αMSH-PEG-Cy5-C′ dots.

FIG. 18L shows a FACS analysis of FSC versus C′ dot Cy5 at 2 days after introduction of 1×PBS to PMA-differentiated THP-1 cells in vitro.

FIG. 19A shows a graph of tissue biodistribution of [²²⁵Ac]αMSH-PEG-Cy5-C′ dots in naive mice (n=3). Data are reported as the mean±standard error of the mean (SEM).

FIG. 19B shows a graph of blood clearance of [²²⁵Ac]αMSH-PEG-Cy5-C′ dots in naive mice (n=3). Data are reported as the mean±standard error of the mean (SEM).

FIG. 19C shows a graph of urinary excretion of [²²⁵Ac]αMSH-PEG-Cy5-C′ dots in naive mice (n=3). Data are reported as the mean±standard error of the mean (SEM).

FIG. 19D shows a graph of tissue biodistribution of [²²⁵Ac]αMSH-PEG-Cy5-C′ dots in tumor-bearing C57BL/6J mice (n=5). Data are reported as the mean±standard error of the mean (SEM).

FIG. 19E shows a graph of blood clearance of [²²⁵Ac]αMSH-PEG-Cy5-C′ dots in tumor-bearing C57BL/6J mice (n=5). Data are reported as the mean±standard error of the mean (SEM).

FIG. 19F shows a graph of urinary excretion of [²²⁵Ac]αMSH-PEG-Cy5-C′ dots in tumor-bearing C57BL/6J mice (n=5). Data are reported as the mean±standard error of the mean (SEM).

FIG. 20A shows a graph of the maximum tolerated dose of [²²⁵Ac]αMSH-PEG-Cy5-C′ dots in naïve C57BL/6J mice (n=5 per group) that received 0, 23.1, 46.3, or 92.5 kBq per mouse.

FIG. 20B shows a graph of alpha particle radiotherapeutic effects on B16-F10 tumor volume.

FIG. 20C shows a graph of alpha particle radiotherapeutic effects on B16-F10 mouse survival.

FIG. 21 shows representative immunofluorescent images of immune cells in the B16-F10 tumor microenvironment.

FIG. 22 shows representative images and graphs of time-dependent increases and decreases of T cells, macrophages, and neutrophils in B16-F10 tumor-bearing mice through the use of staining.

FIG. 23A shows a heat map of top differentially expressed genes in an vehicle-treated control group versus the [²²⁵Ac]αMSH-PEG-Cy5-C′ dot-treated group.

FIG. 23B shows a heat map of top differentially expressed genes in an vehicle-treated control group versus an unlabeled αMSH-PEG-Cy5-C′ dot-treated control group.

FIG. 23C shows a heat map of top differentially expressed genes in the [²²⁵Ac]αMSH-PEG-Cy5-C′ dot-treated group versus an unlabeled αMSH-PEG-Cy5-C′ dot-treated control group

FIG. 24A shows an unsupervised principal component analysis (PCA) showing the first two principal components of all samples using data obtained from RNA-seq.

FIG. 24B shows a heat map of the mean fraction of immune cells signatures in the CD45⁺ cells isolated from individual B16-F10 tumors.

FIG. 24C shows a graph of population changes in T cells, macrophages, monocytes and natural killer cells within the tumor microenvironment as a function of treatment.

FIG. 25A shows a tabular RNA seq data heat map obtained from CIBERSORT and ImmuneCC analysis.

FIG. 25B shows additional tabular RNA seq data heat map obtained from CIBERSORT and ImmuneCC analysis.

FIG. 26A shows a plot of tumor volume measurements over time.

FIG. 26B shows a survival plot of tumor-bearing mice.

FIG. 27 shows a heap map of differentially expressed cytokines.

FIG. 28 shows a schematic of a proposed mechanism of action for a macrophage-initiated, pseudo-pathogenic response to αMSH-PEG-Cy5-C′ dots in the tumor microenvironment.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Definitions

In order for the present disclosure to be more readily understood, certain terms are first defined below. Additional definition for the following terms and other terms are set forth throughout the specification.

About: The term “about”, as used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition comprising a nanoparticle to a subject or system. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous . In certain embodiments, administration is intraperitoneal.

Agent: The term “agent”, as used herein, may refer to a compound, molecule, or entity of any chemical and/or biological class including, for example, a small molecule, polypeptide, nucleic acid, saccharide, lipid, metal, or a combination or complex thereof. In certain embodiments, the term “agent” may refer to a compound, molecule, or entity that comprises a polymer. In certain embodiments, the term may refer to a compound or entity that comprises one or more polymeric moieties. In certain embodiments, the term may refer to a compound, molecule, or entity that lacks or is substantially free of any polymer or polymeric moiety. In some embodiments, the term may refer to a nanoparticle.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CHL CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, an antibody utilized in accordance with certain embodiments of the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with certain embodiments of the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload or other pendant group). In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antigen: The term “antigen”, as used herein, refers to an agent that elicits an immune response; and/or (ii) an agent that binds to a T cell receptor (e.g., when presented by an WIC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, an antigen binds to an antibody and may or may not induce a particular physiological response in an organism.

Antigen presenting cell: The phrase “antigen presenting cell” or “APC,” as used herein, has its art understood meaning referring to cells which process and present antigens to T-cells. Exemplary antigen cells include dendritic cells, macrophages and certain activated epithelial cells.

Biocompatible: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death. In certain embodiments, materials are biodegradable.

Cancer: As used herein, the term “cancer” refers to a malignant neoplasm or tumor (Stedman's Medical Dictionary, 25th ed.; Hensly ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CML, T cell CML), and chronic lymphocytic leukemia (CLL) (e.g., B cell CLL, T cell CLL)); lymphoma such as Hodgkin lymphoma (HL) (e.g., B cell HL, T cell HL) and non Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

Chemotherapeutic Agent: As used herein, the term “chemotherapeutic agent” or “oncolytic therapeutic agent”(e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In many embodiments, chemotherapeutic agents and/or oncolytic therapeutic agents are useful in the treatment of cancer. In some embodiments, a chemotherapeutic agent and/or oncolytic therapeutic agents may be or comprise one or more hormonal agents (e.g., androgen inhibitors), one or more alkylating agents, one or more anthracyclines, one or more cytoskeletal disruptors (e.g., microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinumbased agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, all-trans retinoic acid, an Auiristatin, Azacitidine, Azathioprine, Bleomycin, Bortezomib, Carboplatin, Capecitabine, Cisplatin, Chlorambucil, Cyclophosphamide, curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-PDox, hLL1-Pro-2-P-Dox, P4D1 0-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be or comprise one or more of famesyl-thiosalicylic acid (FTS), 4-(4-Chloro-2-methylphenoxy)-N-hydroxybutanamide (CMI-1), estradiol (E2), tetramethoxystilbene (TMS), δ-tocatrienol, salinomycin, or curcumin. In certain embodiments, chemotherapeutic agents and/or oncolytic therapeutic agents for anti-cancer treatment comprise (e.g., are) biological agents such astumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors) or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-OO1, Bicalutamide, Nilutamide, Flutamide, Enzalutamide, ARN-509).

Marker: A “marker”, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular immune cell type, immune cell subclass, activation of immune cells, and/or polarization of immune cells. Alternatively or additionally, in some embodiments, a presence or level of a particular marker correlates with activity (or activity level) of a particular signaling pathway, for example that may be characteristic of a particular class of immune cells. The statistical significance of the presence or absence of a marker may vary depending upon the particular marker. In some embodiments, detection of a marker is highly specific in that it reflects a high probability that the cell is of a particular immune cell type and/or subclass. In certain embodiments, a marker is a cytokine. In certain embodiments, a marker is a chemokine. In certain embodiments, a marker is a receptor. In certain embodiments, a marker is a genetic marker (e.g., mRNA, RNA) indicative of activation of a gene.

Pharmaceutical composition: As used herein, the term “pharmaceutical composition” refers to an active agent, formulated together with one or more pharmaceutically acceptable carriers. In certain embodiments, active agent is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Radiolabel: As used herein, “radiolabel” refers to a moiety comprising a radioactive isotope of at least one element. Exemplary suitable radiolabels include but are not limited to those described herein. In certain embodiments, a radiolabel is one used in positron emission tomography (PET). In certain embodiments, a radiolabel is one used in single-photon emission computed tomography (SPECT). In certain embodiments, radioisotopes comprise mTc, In, ⁶⁴Cu, ⁶⁷Ga, ¹⁸⁶Re, ¹⁸⁸Re, ¹⁵³sm, ¹⁷⁷Lu, ⁶⁷Cu, ¹²³I, ¹²⁴¹I, ¹²⁵I, ¹¹C, ⁴³N, ¹⁵⁰O, ¹⁸F, ¹⁸⁶Re, ¹⁸⁸Re, ¹⁵³Sm, ¹⁶¹Ho, ¹⁷⁷Lu, ¹⁴⁹Pm, ⁹⁰Y, ²¹³Bi, ¹⁰³Pd, ¹⁰³Pd, ¹⁵⁹Gd, ¹⁴⁰La, ¹⁹⁸AI, ¹⁹⁹AU, ¹⁶⁹Yb, ¹⁷⁵Yb, ¹⁶⁵Dy, ¹⁶⁶Dy, ⁶⁷Cu, ¹⁰⁵Rh , ¹¹¹Ag, ⁸⁹Zr, ²²⁵Ac, ¹⁹²Ir, and ⁸⁹Zr.

Subject: As used herein, the term “subject” includes humans and mammals (e.g., mice, rats, pigs, cats, dogs, and horses). In many embodiments, subjects are mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

Therapeutically effective amount: as used herein, is meant an amount that produces the desired effect for which it is administered. In certain embodiments, the term refers to an amount that is sufficient, when administered to a population suffering from or susceptible to a disease, disorder, and/or condition in accordance with a therapeutic dosing regimen, to treat the disease, disorder, and/or condition. In certain embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In certain embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in certain embodiments, a therapeutically effective amount of a particular agent or therapy may be formulated and/or administered in a single dose. In certain embodiments, a therapeutically effective agent may be formulated and/or administered in a plurality of doses, for example, as part of a dosing regimen.

Therapeutic agent: As used herein, the phrase “therapeutic agent” in general refers to any agent that has a therapeutic effect and/or elicits a desired biological and/or pharmacological effect when administered to a subject.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments as discussed herein, a tumor is associated with, or is a manifestation of, a cancer. In some embodiments as discussed herein, a tumor may be a solid tumor.

Drawings are presented herein for illustration purposes, not for limitation.

### DETAILED DESCRIPTION

It is contemplated that methods, compositions, and processes of the claimed invention encompass variations and adaptations developed using information from the embodiments described herein. Adaptation and/or modification of the methods, compositions, and processes described herein may be performed, as contemplated by this description.

Throughout the description, where methods, compositions, and processes are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

Experiments with ultrasmall silica nanoparticles demonstrate favorable activation of the tumor microenvironment (e.g., macrophages, T cells, and antigen-presenting cells (APCs, such as dendritic cells)). These effects may be beneficial, for example, in checkpoint inhibition therapy (e.g., anti-PD1) or radiotherapy, or a combination of both radiotherapy and checkpoint inhibitors. From the experiments described herein, it is also presently found that it is possible to activate the tumor microenvironment with “cold” particles without a targeting moiety. Experiments conducted either without or with a targeting moiety attached (e.g., PEG-C′ dots vs. αMSH-bound C′ dots) each resulted in activation of the tumor microenvironment.

Without wishing to be bound to any particular theory, iron entrained within the pores of particles, and used at low concentrations, initiate pro-inflammatory responses, while much higher particle concentrations do not (i.e., high concentrations are needed to drive ferroptotic induction). Further experiments described herein are designed to test this mechanism by blocking iron uptake. In vivo experiments are also are used to determine whether T cell activation arises following particle injection, e.g., cytotoxic T cells.

In certain embodiments, the nanoparticle is or comprises an inhibitor-functionalized ultrasmall nanoparticle as described in International Patent Application No. PCT/US17/63641, “Inhibitor-Functionalized Ultrasmall Nanoparticles and Methods Thereof,” filed Nov. 29, 2017, published as WO/2018/102372, the text of which is incorporated herein by reference in its entirety. In certain embodiments, the nanoparticle has from 1 to 100 targeting ligands (e.g., from 1 to 80, e.g., from 1 to 60, e.g., from 1 to 40, e.g., from 1 to 30, e.g., from 1 to 25 targeting ligands) attached thereto. In certain embodiments, the targeting ligands comprise alpha-MSH. In certain embodiments, the nanoparticle has an average diameter of no greater than about 50 nm (e.g., no greater than about 40 nm, e.g., no greater than about 30 nm, e.g., no greater than about 25 nm, e.g., no greater than about 20 nm, e.g., no greater than about 10 nm, e.g., no greater than about 8 nm).

In certain embodiments, the nanoparticle is administered in a combination therapy and/or along with ferroptotic inhibiting agents as described in International Patent Application No. PCT/US18/63751, “Methods of Cancer Treatment via Regulated Ferroptosis,” filed Dec. 4, 2018, published as WO/2019/113004, the text of which is incorporated herein by reference in its entirety. In certain embodiments, the method comprises administering one or more regulators of ferroptosis. In certain embodiments, the one or more regulators of ferroptosis comprise one or more one or more inhibitors of ferroptosis. In certain embodiments, the regulator of ferroptosis is an inhibitor of ferroptosis. In certain embodiments, the one or more inhibitors of ferroptosis comprises a member selected from the group consisting of liproxstatin-1, ferrostatin-1, and/or other compounds which scavenge lipid peroxides.

In certain embodiments, nanoparticles herein comprise a silica core and shell. In certain embodiments the diameter of the nanoparticle core ranges from 1 to 20 nm, from 1.5 to 20 nm, from 2 to 8 nm. In certain preferable embodiments, the diameter of the nanoparticles range from 2 to 6 nm. In certain embodiments, the nanoparticle shell thickness is less than 5 nm,

In certain embodiments, the nanoparticles have an ability to target cancerous tissues and/or cells. In certain embodiments, the nanoparticles comprise 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more ligands. In certain preferable embodiments, the nanoparticle comprises at least 5 ligands. In certain preferable embodiments, the nanoparticle comprises no more than 15 ligands. In certain embodiments, nanoparticles comprises one or more ligands for targeting a cellular receptor (e.g., MC1-R, MSHR). In certain embodiments, the one or more ligands comprise a peptide (e.g., a-melanocyte stimulating hormone (αMSH)).

In certain embodiments, the nanoparticle comprises hydrophobic surface patches. In certain embodiments, the nanoparticle comprises 0, 1, 2, 3, 4, 5, 6, 7 or more hydrophobic surface patches. In certain preferable embodiments, the nanoparticle comprises 0 hydrophobic surface patches. In certain preferable embodiments, the nanoparticle comprises 4 hydrophobic surface patches.

In certain embodiments, the particles induce the death of cells (e.g., cancer cells) through ferroptosis. In certain embodiments, the particles do not induce ferroptosis in cells.

In certain embodiments, the nanoparticles accumulate tumors. In certain embodiments, the nanoparticles accumulate in primary tumors and/or metastatic tumors. In certain embodiments, the nanoparticles accumulate in melanomatous lesions.

In certain embodiments, nanoparticles comprise fluorescent core-shell silica particles.

In certain embodiments, nanoparticles may be internalized (e.g., phagocytosed) within one or more cell types (e.g., macrophages, THP-1 cells, cancer cells, e.g., B16-F10 cells).

In certain embodiments, nanoparticles one or more ligands may have high binding affinities. In certain embodiments, the binding affinity may be less than 100 nM, less than 50 nM, less than 10 nM.

In certain embodiments, the nanoparticles demonstrate relatively rapid renal clearance. In certain embodiments, the nanoparticles do not induce a toxic response in non-tumor tissue (i.e., normal tissue).

In certain embodiments, the nanoparticles induce tumor regression. In certain embodiments, nanoparticles augment checkpoint blockade.

In certain embodiments, nanoparticles are directed to and/or accumulate in the tumor microenvironment. In certain embodiments, nanoparticles target and/or activate immune cells. In certain embodiments, nanoparticles induce M1 pro-inflammatory phenotype. In certain embodiments, nanoparticles inhibit M2 anti-inflammatory phenotype. In certain embodiments, nanoparticles do not induce ferroptosis.

In certain embodiments, nanoparticles may be imaged using an imaging technique (e.g., fluorescent imaging, MRI, PET/CT imaging, PET imaging, e.g., ⁸⁹Zr PET imaging).

In certain embodiments, the provided compositions comprising nanoparticles are useful in medicine.

### Nanoparticle Induction of Immune Response in the Tumor Microenvironment

In certain embodiments as discussed herein, ultrasmall fluorescent core-shell silica nanoparticles (e.g., C′ dots, C dots) have therapeutic capabilities. In certain embodiments, the nanoparticles allow for a distinct combination of activities that: target cancer cells directly for cell death through the mechanism ferroptosis and/or modulate immune cells directly for polarization toward a pro-inflammatory phenotype. In certain embodiments, a nanoparticle-based agent that can directly induce cancer cell death (e.g., through ferroptosis), in addition to activating and/or priming immune cells through separable activities.

Moreover, among efforts to identify mechanisms of cell death with relevance to human disease, ferroptosis has emerged as a form of cell death with a unique property that promotes the spreading of cell death throughout cell populations (e.g., within a tumor environment, within tumors, within cancer cell populations), an activity that is of clinical significance for eliminating cancerous lesions. In certain embodiments, nanoparticles (e.g., C dots, C′ dots) have a unique ability to engage this form of cell death, underscoring a further innovative aspect of the proposed work that seeks to leverage a unique death-inducing activity for cancer therapy.

In certain embodiments, the surface chemical properties of nanoparticles are characterized (e.g., using high-performance liquid chromatography (HPLC), using gel-permeation chromatography (GPC)). In certain embodiments, GPC is used to characterize the size dispersity of nanoparticles. In certain embodiments, characterization of nanoparticles may be used, in part, to determine an immune response.

### Synthesis of Nanoparticles

In certain embodiments, nanoparticles comprising C′ dots are synthesized as discussed herein.

PEGylated, Cy5-dye encapsulating and αMSH-ligand bearing targeted fluorescent core-shell silica nanoparticles (e.g., αMSH-PEG-Cy5-C′ dots) together with their non-targeted (PEG-Cy5-C′ dots) controls, is synthesized in an aqueous solution.

Cy5-maleimido derivatives is first coupled to a mercapto-silane to form a dye-silane conjugate. The dye-silane conjugate is subsequently co-condensed with TMOS in aqueous solutions at basic pH to form the Cy5 dye-encapsulating silica core. In certain embodiments, silica particle growth is quenched at appropriate time intervals to control silica core size by adding either monofunctional PEG-silane (6-9 EO units per chain), resulting in untargeted PEG-Cy5-C′ dots. In certain embodiments, first hetero-bifunctional PEG, functionalized on one end with a silane and on the other with αMSH peptide, immediately followed by monofunctional PEG-silane, is used to quench the reaction. In certain embodiments, the nanoparticle comprises αMSH ligands. In certain embodiments, ligand density is varied between 5 and 15 ligands per particle by adding increasing amounts of heterobifunctional PEG to the growing silica cores. In certain embodiments, subsequent purification from unreacted precursors and/or particle aggregates is performed using gel permeation chromatography (GPC).

In certain embodiments, nanoparticles as discussed herein are characterized using a particle characterization technique (e.g., FCS, DLS, zeta-potential, UV-VIS absorption, emission spectroscopy, transmission electron microscopy).

For example, fluorescence correlation spectroscopy (FCS) determines particle hydrodynamic size and concentration. Dynamic light scattering (DLS) and/or zeta-potential measurements determine hydrodynamic size and/or surface charge. UV-VIS absorption and emission spectroscopy determine a number of dyes and/or αMSH ligands per particle (e.g., in conjunction with FCS). Transmission electron microscopy (TEM) determines silica core size.

### Controlling Hydrophobic Particle Surface Patchiness

In certain embodiments, hydrophobic “patchiness” of nanoparticles is controlled using the methods and techniques described herein. The surface patchiness of the nanoparticles is used to control, among other things, tumor microenvironment response to nanoparticles.

In certain embodiments, two Cy5-maleimido dye derivatives with different net charges are used: negatively charged sulfo-Cy5(-)-maleimide dye (GE) or positively charged Cy5(+)-maleimide dye (Lumiprobe). As a result of Coulombic interactions with negatively charged ˜2 nm sized silica clusters, initially formed in the sol-gel synthesis of silica, negatively charged sulfo-Cy5 dye preferentially ends up on the silica core surface, while positively charged Cy5 can be fully encapsulated.

In certain embodiments, control over the surface patchiness can be exerted by controlling the number of Cy5 dyes on the surface of the silica core of a nanoparticle by using different concentrations of ammonia as sol-gel catalyst. In certain embodiments, there are between zero and four Cy5 dyes on the silica core surface. In certain embodiments, patchiness has an effect on ferroptosis induction. In certain embodiments, patchiness has an effect on immune cell priming and/or activation. Hydrophobic patchiness from Cy5 dyes ending up on the C′ dot surface can be verified by HPLC. For example, a HPLC using 150 mm Waters Xbridge BEH C4 protein separation columns with 300 Å pore size and 3.5 μm particle size, and a water/acetonitrile mixture as mobile phase may be used.

### Controlling Silica Core and PEG/Ligand Shell Size

In certain embodiments, the synthesis of the silica core of the nanoparticle is controlled as described herein. The water-based synthesis of C′ dots enables control of the silica core size at the level of a single atomic SiO₂ layer. As described herein, the exceptional degree of particle size control allows generation of nanoparticles (e.g., C′ dots) with overall particle size maintained below the cut-off for renal clearance (e.g., below 15 nm) to reduce unwanted off-target accumulations (e.g., in the liver), while varying sizes of core and/or shell. Silica core size is reduced by increasing reaction temperature and/or by decreasing the time of core growth before PEG-silane is added. In certain embodiments, the length of the PEG-silane chains (Gelest) is increased to maintain an overall hydrodynamic size of the nanoparticle. Changing relative sizes of silica core and/or PEG shell of otherwise same hydrodynamic size of nanoparticles (e.g., C′ dots) allows decoupling contributions of silica core and PEG shell to ferroptosis and/or immune cell priming.

### Controlling Silica Core Composition

In certain embodiments, the silica core composition of nanoparticles is modulated as described herein. In certain embodiments, modulation of the composition of the silica cores affects affinity of iron to C′ dots, which will be chelated by silanol (—SiOH) surface groups in micropores of the sol-gel derived silica core. Silica core composition can be varied, e.g. by the addition of aluminum sec-butoxide, mercapto-silane, and/or iodo-silane moieties into the aqueous sol-gel reaction mixture.

In certain embodiments, affinity of iron to a silica core is modulated through phosphonate-silane conjugates co-condensed with TMOS in the silica core synthesis. Phosphonates are known for their high affinity to metal ions like iron. Beyond about 15 mole% of phosphonate-silane in the reaction, relative to TMOS, the effect of ferroptosis on amino-acid-deprived MDA-MB-468 TNBCs at C′ dot concentrations of 15 μM is essentially switched off. Without wishing to be bound to any particular theory, this is due to the high affinity of iron to the phosphonate groups and related reduction of iron release once the iron-loaded particles are internalized by cells. In certain embodiments, phosphonate group bearing C′ dots effect ferroptosis and/or immune cell priming and/or activation. In certain embodiments, microwave plasma atomic emission spectroscopy is used to evaluate nanoparticle iron concentrations. These nanoparticles help delineate molecular mechanisms by which C′ dots induce ferroptosis and/or activation of immune cells.

### Example 1: Induction of Tissue Microenvironment Changes in Melanoma Tumor-Bearing Models Using C′ Dots

In certain embodiments, nanoparticles as disclosed herein (e.g., C′ dots) inhibit tumor growth and/or induces tumor regression.

For example, intravenous (i.v. or IV) administration of 60 μM of stock C′ dots (36 nmoles in total) to mice bearing 786-O renal carcinoma xenografts inhibits tumor growth and leads to regression of HT1080 fibrosarcoma tumors, but has no toxic effects on normal tissues as shown by complete blood counts, serum chemistry, and histopathology.

Regression of HT1080 xenografts by C′ dots was blocked by co-injection of liproxstatin-1, a specific inhibitor of ferroptosis as ferroptosis is known to occur in response to the accumulation of intracellular iron. Furthermore, a resulting increase in reactive oxygen species leads to lipid peroxidation and cell membrane rupture. In addition, macrophages are recruited to C′ dot-treated tumors. This demonstrates that C′ dots also engage immune responses during HT1080 tumor regression.

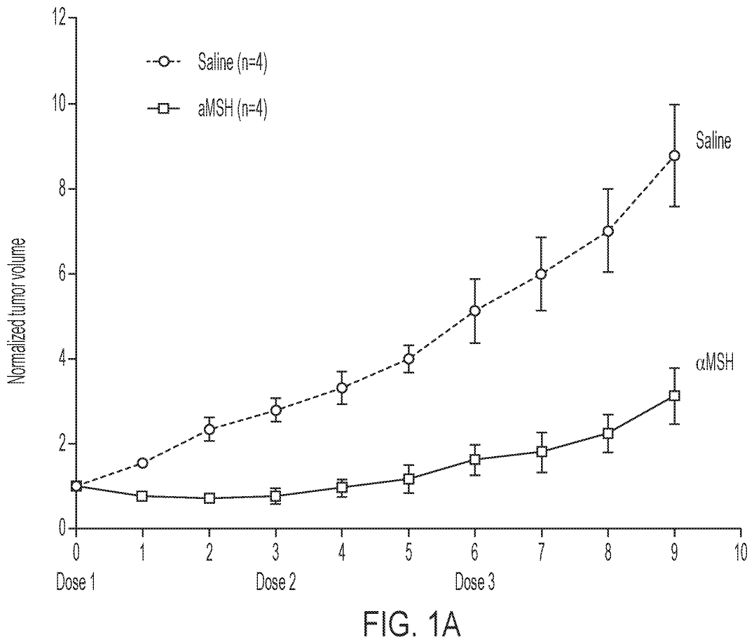

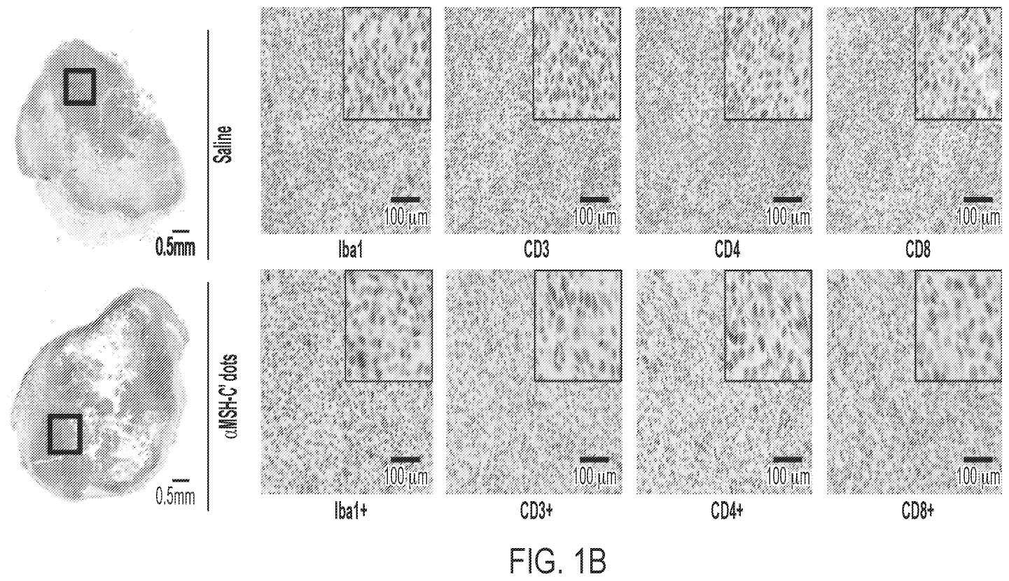

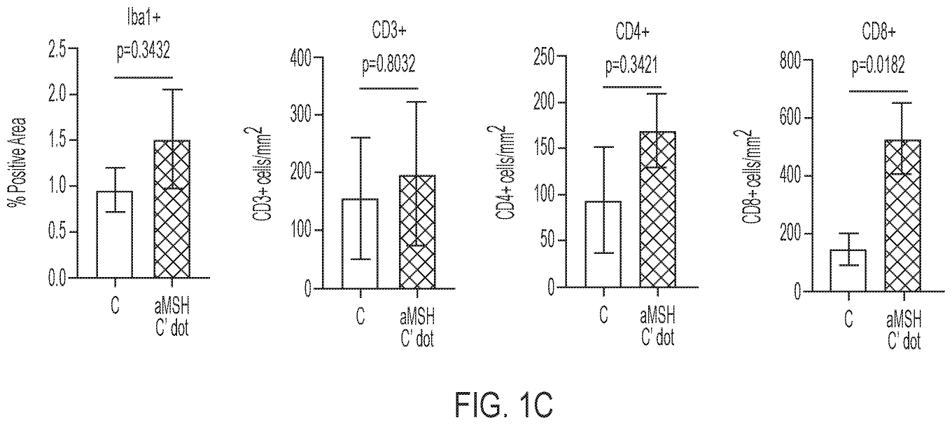

In certain embodiments, C′ dot administration inhibits the growth of B16-F10 melanoma as seen in FIG. 1A-C. In FIG. 1A, tumor growth inhibition can be seen over a period of 9 days after implantation when comparing the normalized tumor volume of mice having been administered αMSH-C′ dots (αMSH-PEG-C′ dots is used interchangeably herein with αMSH-C′ dots) to mice having been administered saline vehicle.

Mice (n=4 at each data point) with a B16-F10 xenografted tumor are administered either saline vehicle (top line, green line) or αMSH-C′ dots (bottom line, blue line) at 0 days, 3 days, and 6 days after implantation of the tumor. Each dose of αMSH-C′ dots is 36 nmoles of αMSH-C′ dots having been administered to a mouse via i.v. injection from a 60 μM stock of αMSH-C′ dots in saline. Each data point in FIG. 1A is representative of the mean normalized tumor volume of 4 mice.

In addition, FIG. 1B shows histological sections of B16-F10 xenografted tumors. Representative images of tumors from mice having been administered saline (top row) or αMSH-C′ dots (bottom row) are shown. These tumors were obtained after 10 days.

The histological sections of FIG. 1B show alterations in immunogenic cell populations through the use of antibody markers and red chromagen. Without wishing to be bound to any specific theory, the presence of these antibody markers in the tumor microenvironment indicate the presence of particular cell populations. These cell populations testing positive for each of the respective markers include macrophages (i.e., Iba1+), pan T cells (i.e., CD3+), helper T cells (i.e., CD4+), and cytotoxic T cell populations (i.e., CD8+). The tumor exposed to αMSH-C′ dots shows a general increase in these aforementioned populations of cells as shown in the graphs of FIG. 1C. FIG. 1C shows histograms indicating the percentage of positive area or number of cells per area for each of the aforementioned cell populations in a tumor of a mouse having been treated with saline vehicle (‘C’) or αMSH-C′ dots (αMSH C′ dot). Of particular note, there is a statistically significant increase in the number of CD8+ T cells per unit area in the tumor microenvironment (TME).

### Example 2: Induction of Macrophage Changes in In Vitro Co-Culture Models

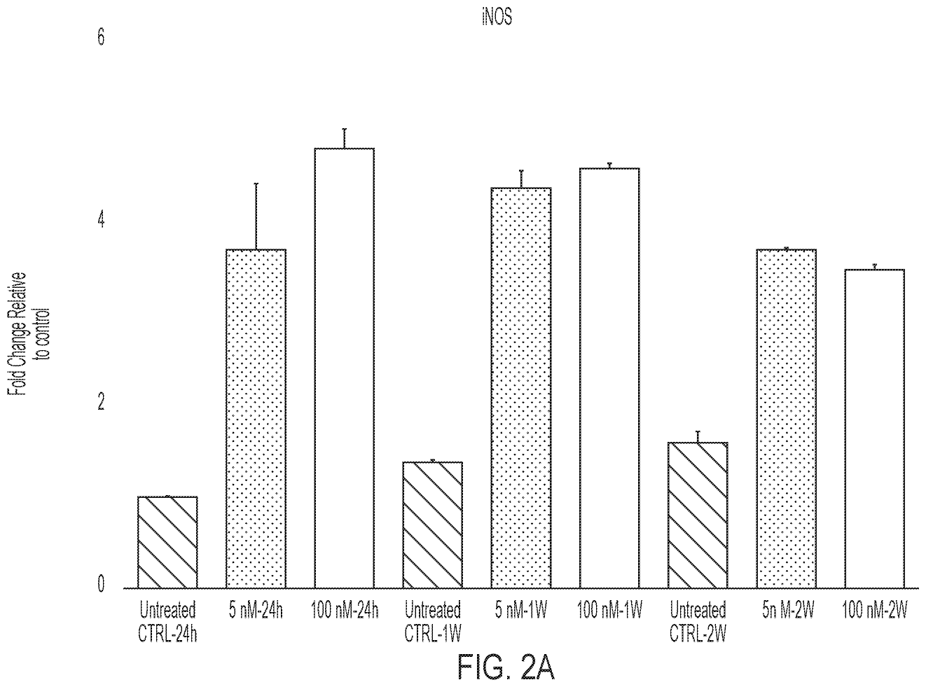

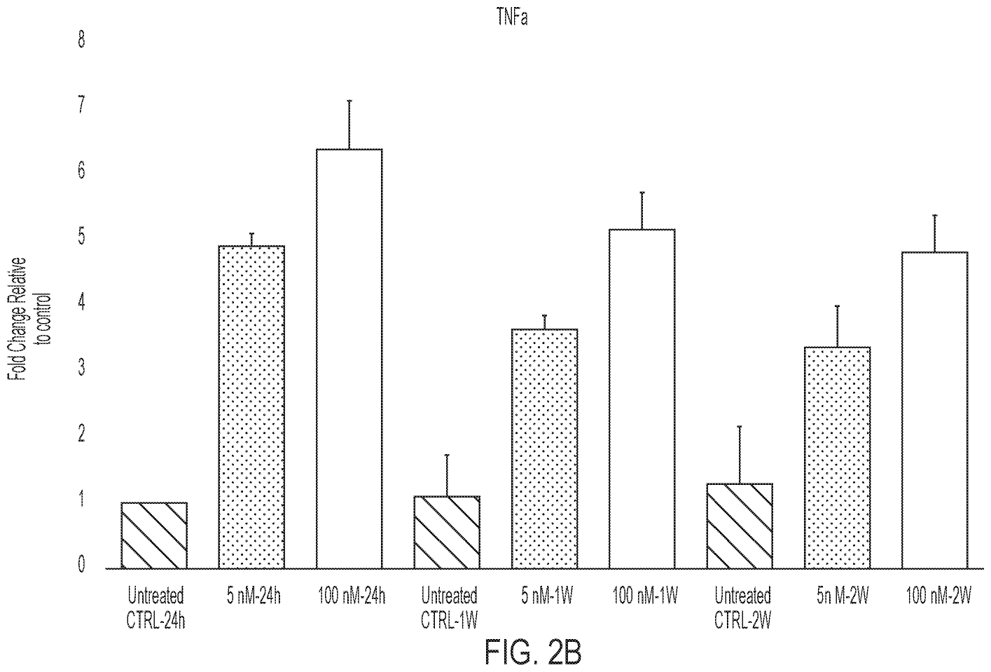

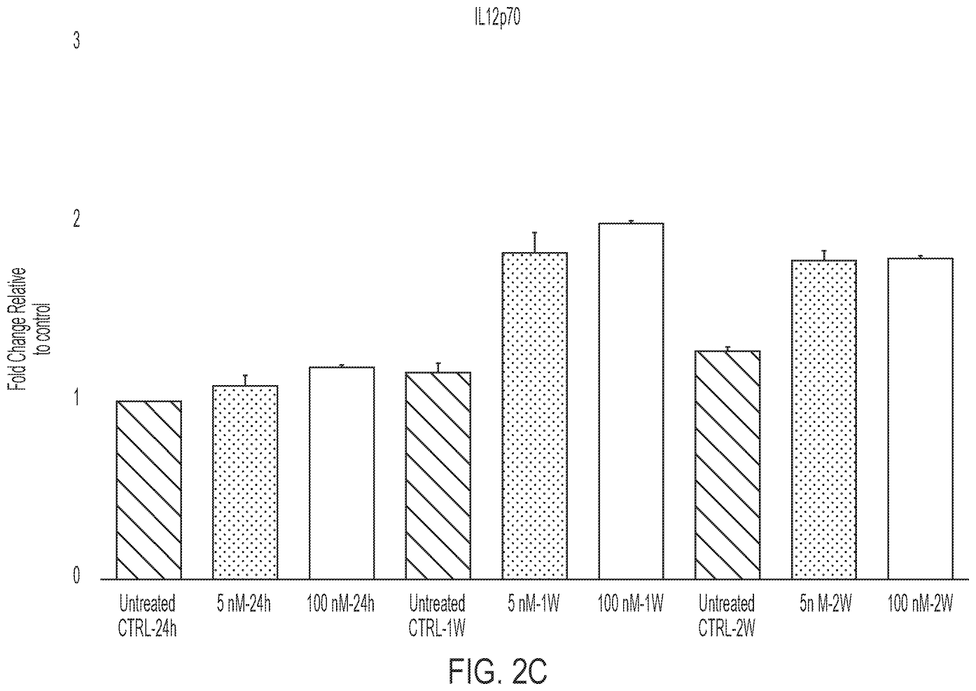

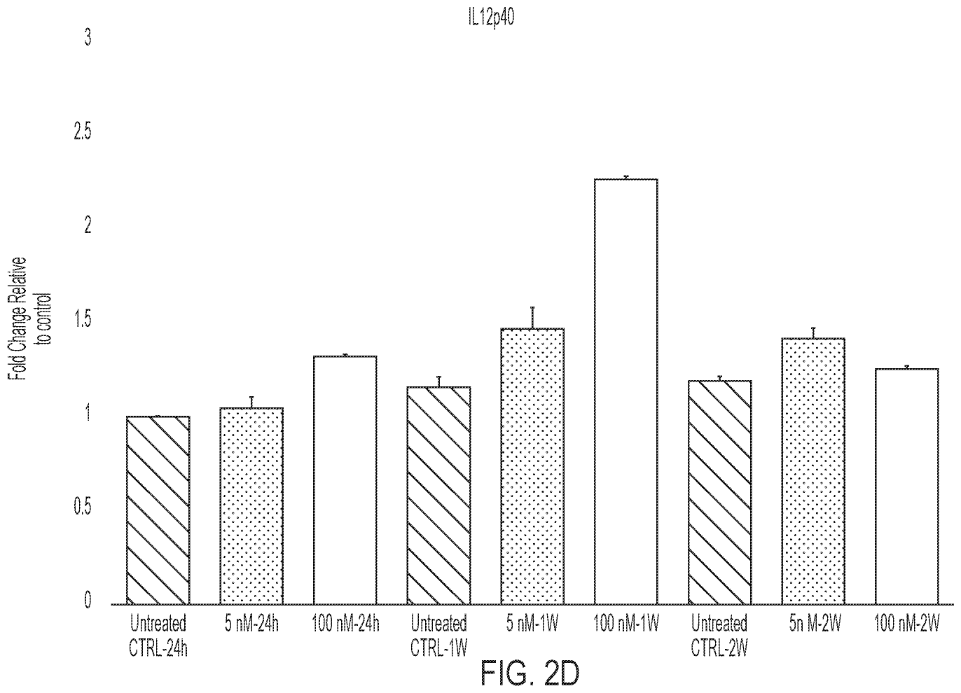

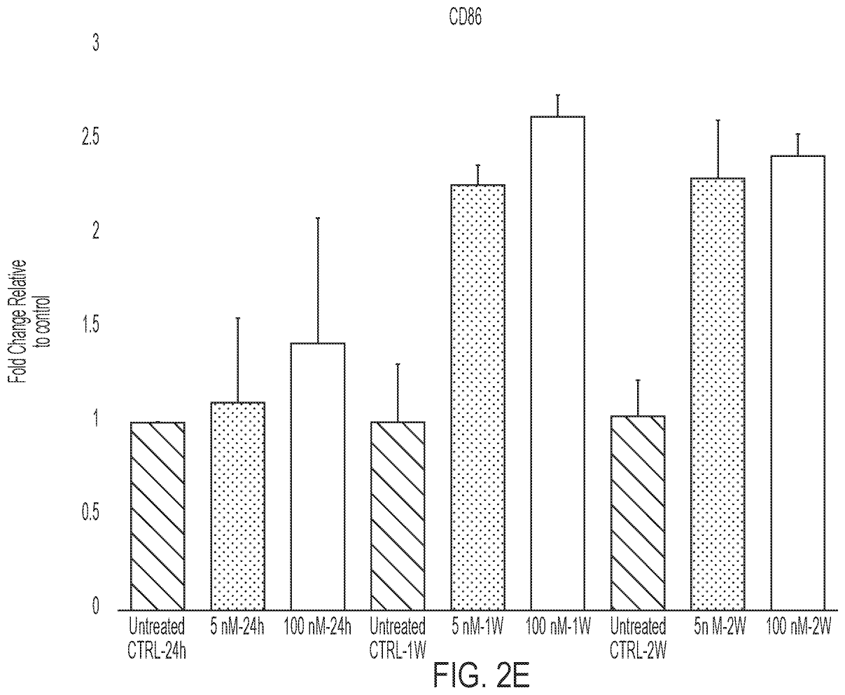

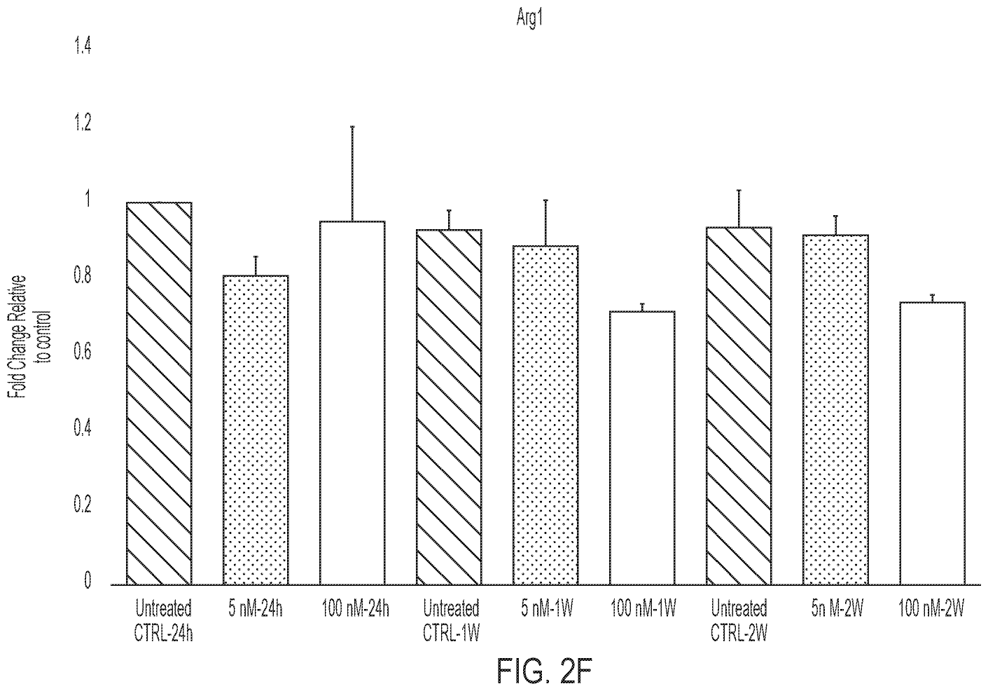

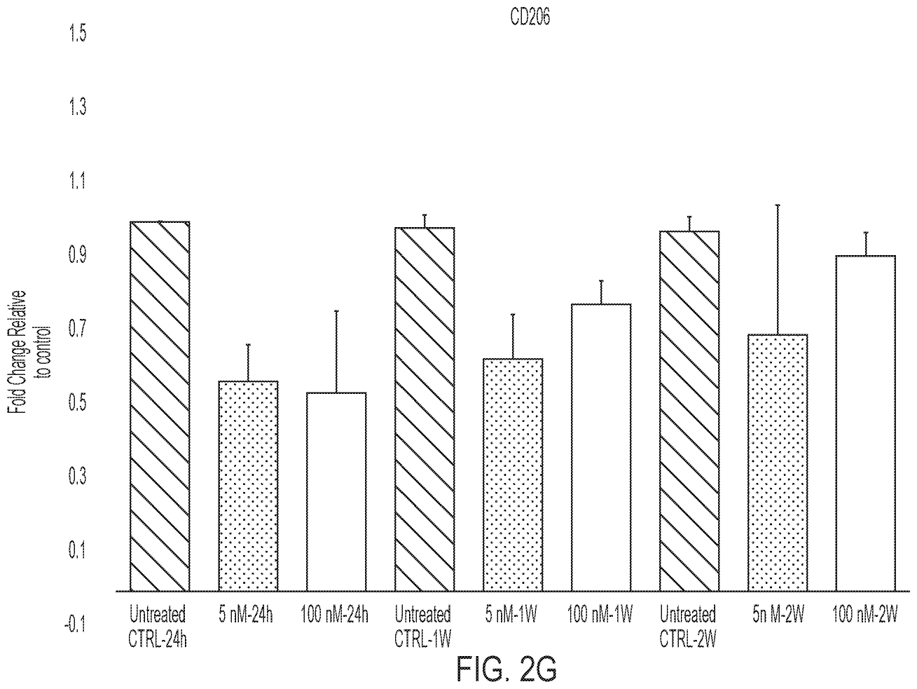

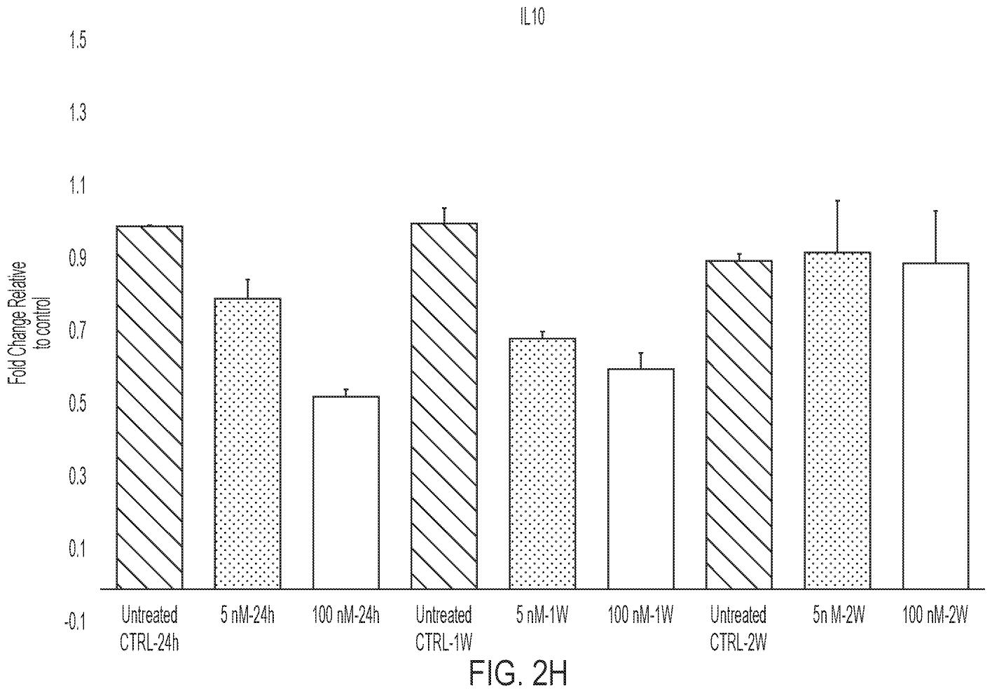

The experiments in FIGS. 2A-H demonstrate changes in the gene expression profiles of mouse bone marrow-derived macrophages (BMDMs) treated with low dosages of C′ dots. Treatment with low dosages of C′-dots is seen to increase pro-inflammatory, anti-tumor markers, while decreasing pro-tumor markers. Accordingly, C′-dots are indicative of the induction of a pro-inflammatory tumor microenvironment.

Mouse bone marrow derived macrophages (BMDMs) treated with either 5 nM C′ dots or 100 nM C′ dots show signs of pro-inflammatory macrophage activation over the course of 1 (24 h), 7 (1 W) and 14 (2 W) days, as measured by quantitative reverse transcription polymerase chain reaction (qRT-PCR). In FIGS. 2A-E, iNOS (FIG. 2A), TNFα (FIG. 2B), IL12p70 (FIG. 2C), IL12p40 (FIG. 2D), and CD86 (FIG. 2E) are markers associated with M1 type, pro-inflammatory macrophages. These markers are generally seen to increase after treatment with C′ dots and are indicative that a pro-tumor microenvironment may be created in vivo as well.

In contrast, the gene expression of Argl (FIG. 2F), CD206 (FIG. 1G), and IL10 (FIG. 1H) are seen to decrease under treatment using C′ dots as compared to the control, untreated cells at similar time points. These aforementioned markers are generally indicative of M2 type, pro-tumor macrophages. Accordingly, treatment with C′-dots is associated with creating a tissue microenvironment that is less tumorigenic.

### Example 3: Induction of Macrophage Changes in In Vitro Co-Culture Models

While other nanoparticle platforms elicit immune cell responses, these generally involve large-particle (e.g., 30-100 nm) delivery of exogenous cytokines, antigens, or Toll-like receptor (TLR) agonists. Other nanoparticles with intrinsic activity have been shown to engage complement activation or damage endosomes, thereby inducing oxidative stress and cell death after uptake (e.g., through ferroptosis). These mechanisms do not result in nanoparticle immune effects as discussed herein. In certain embodiments, the response of cells to administration of nanoparticles does not induce cellular dysfunctions (e.g., lysosome dysfunction) and cell death.

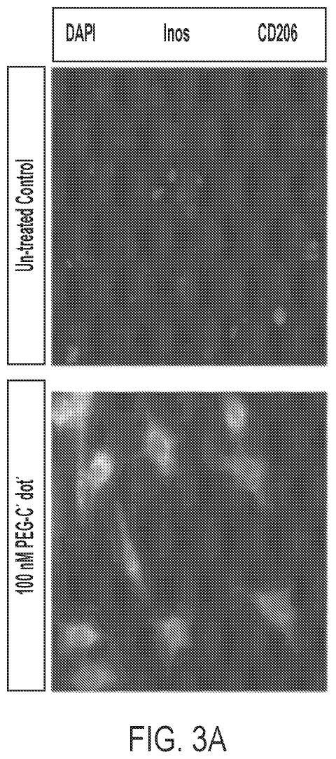

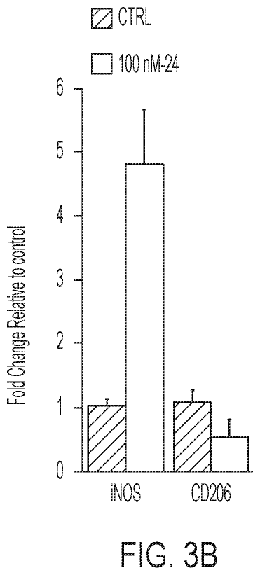

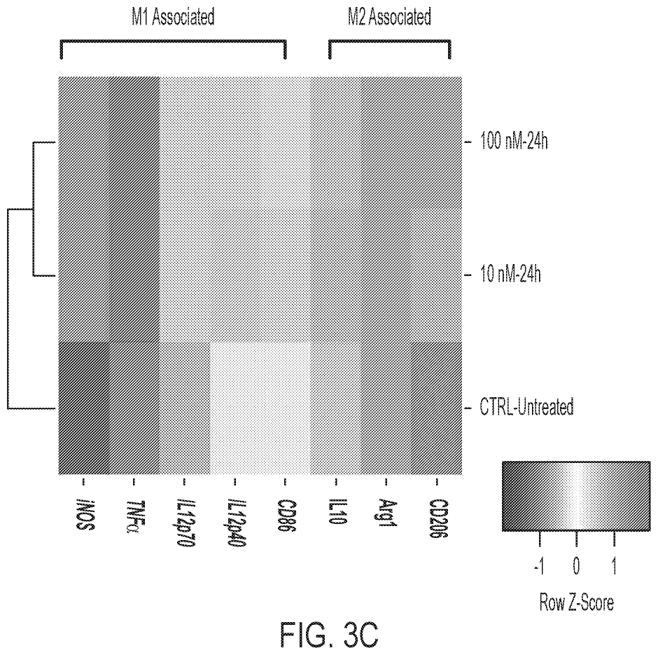

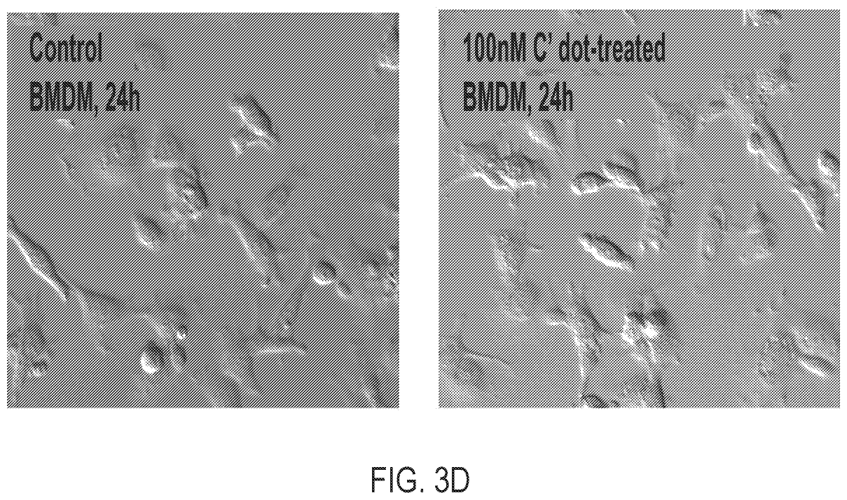

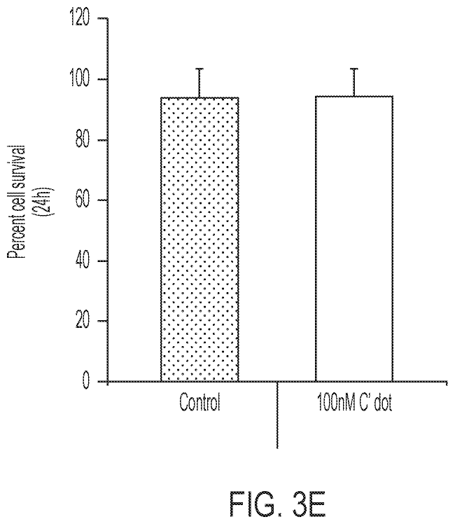

In certain embodiments, nanoparticles (e.g., C′ dots) are directly delivered to cells. For example, direct C′ dot delivery to macrophages results in M1 macrophage polarization in a ferroptosis-independent manner (e.g., see FIGS. 3A-E), demonstrating that C′ dot treatment directly regulates macrophage phenotypes in the tumor microenvironment (TME). For immune cells, treatment with low-dose particle concentrations (e.g., 10 nM or 100 nM) are sufficient to polarize macrophages, but do not induce ferroptosis of cells in the tumor microenvironment.

For example, in FIGS. 3A-E, mouse bone marrow-derived macrophages (BMDMs) treated with either 10 nM or 100 nM PEG-C′ dots for 24 hours show upregulation of M1 polarization markers (iNOS and TNFα) and downregulation of M2 markers (CD206, Argl and IL-10). The upregulation of M1 associated polarization markers are indicative of a pro-inflammatory TME.

FIG. 3A shows immunofluorescent staining of BMDMs treated with 100 nM PEG-C′ dots or BMDMs not having been treated with PEG-C′ dots. Cells were stained using DAPI for nuclei and markers for iNOS (green) and CD206 (red) indicative of macrophage polarization. An increase in iNOS is indicative of M1 macrophage polarization, while an increase in CD206 is indicative of M2 macrophage polarization. As can be seen from the immunofluorescence image of FIG. 3A, the relative amount of iNOS (green) staining increases with the treatment of a low dosage of PEG-C′ dots, while the relative amount of CD206 (red) does not significantly change with the treatment. Accordingly, a low dosage of PEG-C′ dots has been found to induce M1 polarization. Furthermore, FIG. 3B shows a graph demonstrating similar results using qRT-PCR. iNOS is also upregulated and CD206 is downregulated in BMDMs treated with 100 nM PEG-C′ dots for 24 h as compared to untreated BMDMs over the same time period.

FIG. 3C is a heat map of M1 and M2 associated polarization markers when cells are treated with low dosages of PEG-C′ dots. The M2 markers show a decrease in expression when cells are treated with either 100 nM PEG-C′ dots for 24 hours or 10 nM PEG-C′ dots for 24 hours, as compared to untreated cells. In addition, the M1 associated markers iNOS and TNFα are seen to increase (blue) with treatment of low doses of either 100 nM PEG-C′ dots for 24 hours or 10 nM PEG-C′ dots for 24 hours, as compared to untreated cells.

The treatment of BMDMs with low dosages of PEG-C′ dots also does not induce cell death as can be seen in FIGS. 3D-E. In FIG. 3D, DIC images show control (left) and treated (right) BMDMs taken at 24 h from a time-lapse sequence. Cells from both images appear to be healthy and show no obvious phenotypic differences.

Furthermore, treatment with low dosages of PEG-C′ dots was found to not have an effect on overall cell survivability over 24 h. FIG. 3E shows the average percent cell survival for control (black) and 100 nM PEG-C′ dot treated (gray) BMDMs after 24 h. Data in FIG. 3E show the average percentage of surviving cells out of 15 microscopic fields of view, wherein each field of view was taken from a separate, independent experiment. As shown in FIG. 3E, percent cell survival over 24 h does not significantly change when cells are treated with 100 nM PEG-C′ dots for 24 hours as compared to control cells.

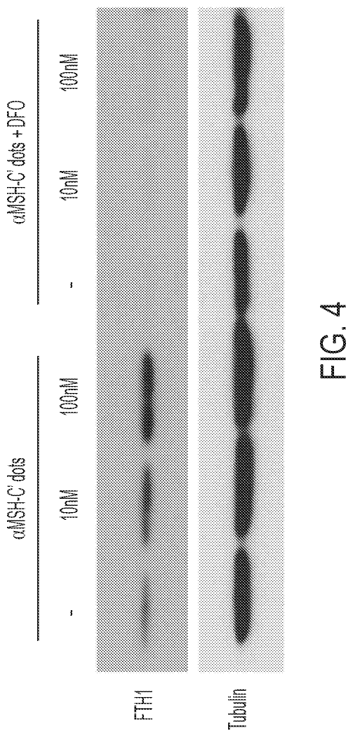

Without wishing to be bound to any particular theory, in certain embodiments iron delivery by C′ dots into macrophage lysosomes induces M1 polarization as indicated by changes in marker expression profiles as seen herein. When cells are exposed to αMSH-C′ dots in low concentrations, BMDMs upregulate ferritin heavy chain (FTH1) as can be seen in the left half of FIG. 4. The increase in FTH1 is consistent with an increased level of intracellular iron due to particle-mediated delivery of iron. To verify this mechanism of action, αMSH-C′ dots were also administered to BMDMs along with DFO (Deferoxamine), an iron chelator, as can be seen in the right half of FIG. 4. The administration of the iron chelator is correlated with a lack of FTH1 expression in αMSH-C′ dot treated and untreated BMDMs. Accordingly, as evidenced herein, an activity such as iron loading may underlie the ability of C′ dots to polarize M1 immune cells in the tumor microenvironment in the absence of ferroptosis or ferroptotic conditions.

### Example 4: C′ Dot-Induced Changes in Cytokine Expression Profile In Vitro

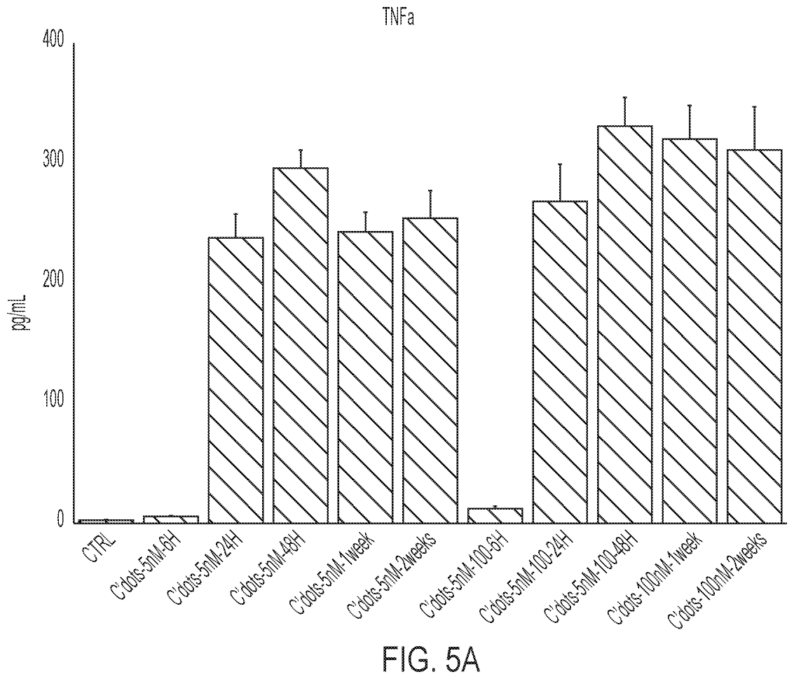

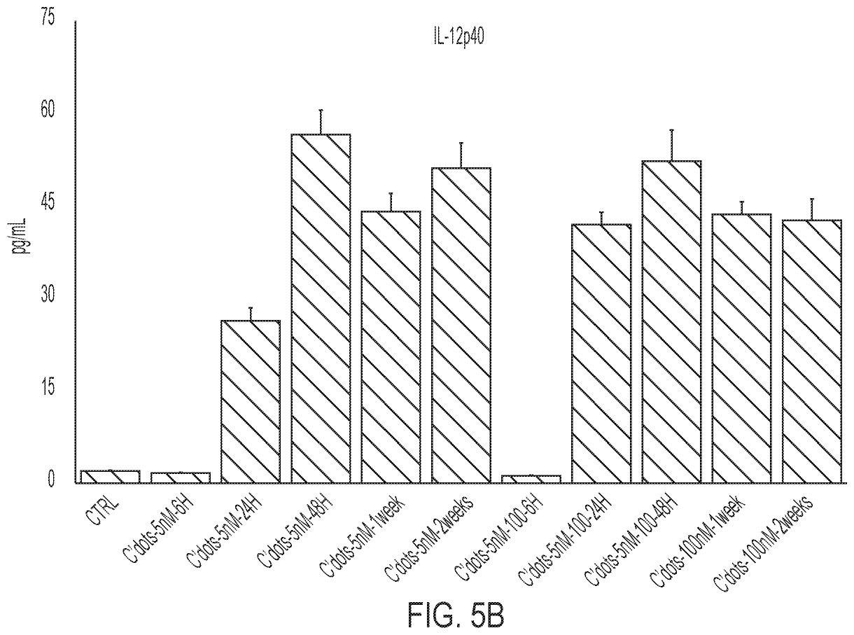

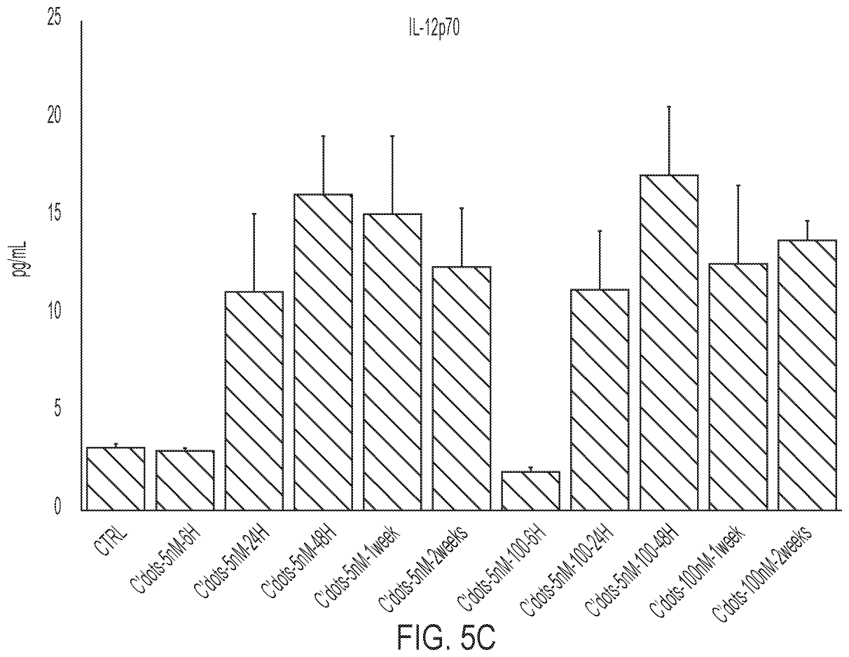

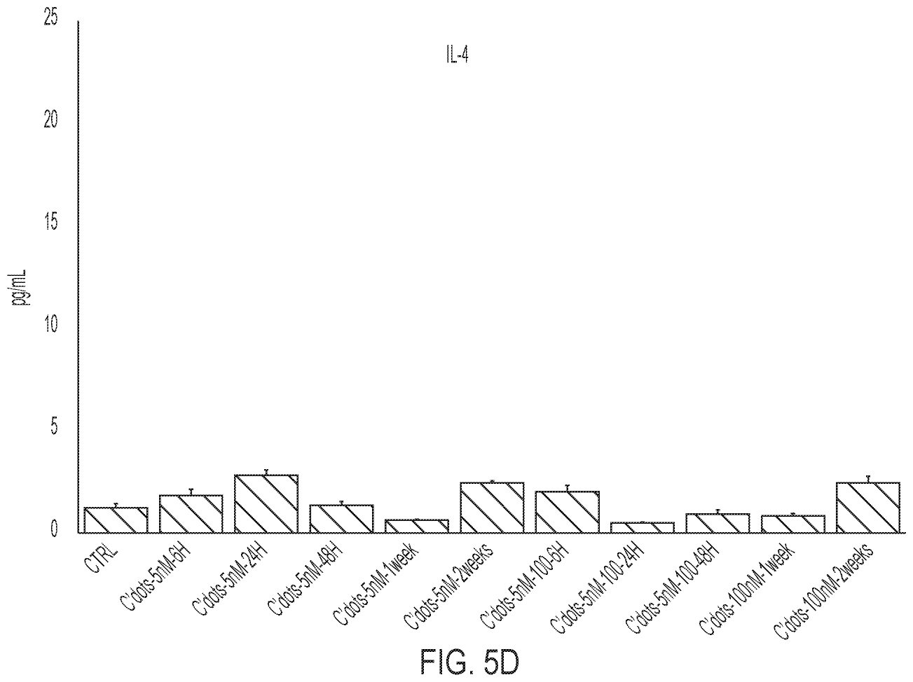

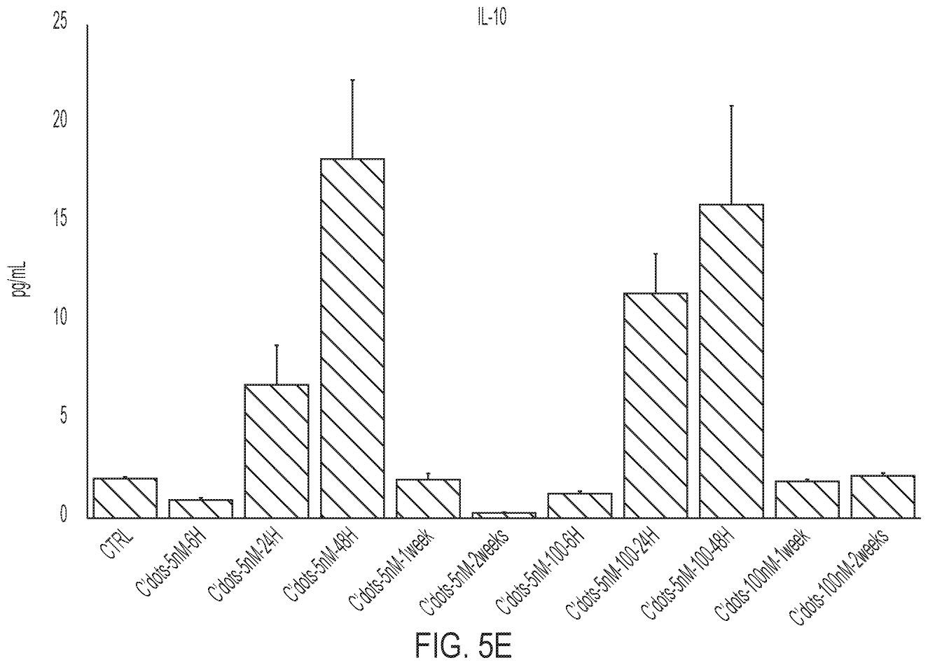

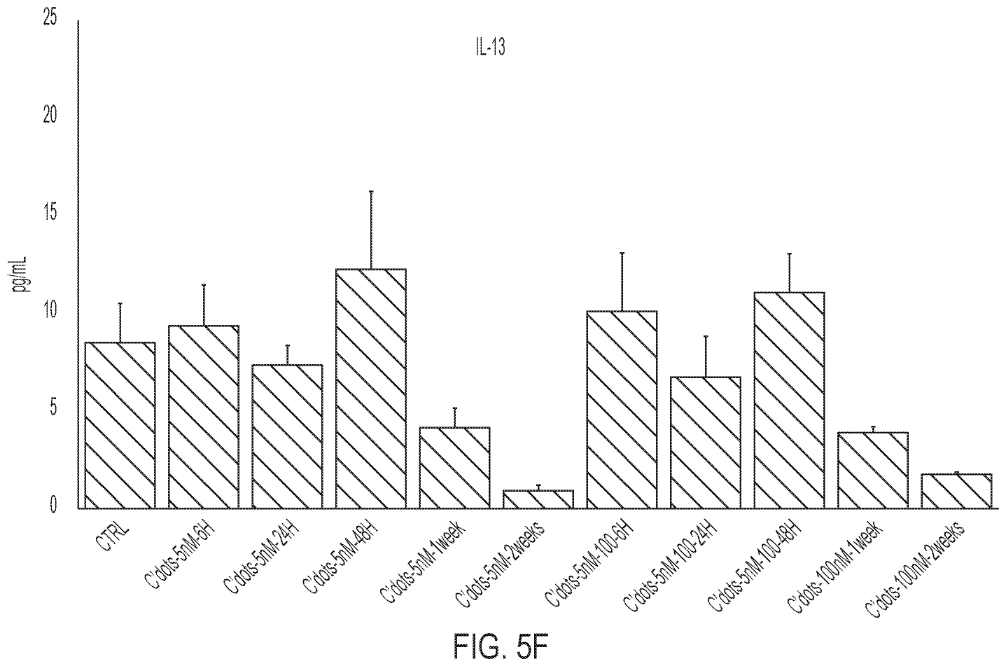

FIGS. 5A-F show changes in the cytokine release profiles in BMDMs exposed to low doses of PEG-C′ dots in vitro for time periods of up to 2 weeks. The change in cytokine expression profiles of PEG-C′ dot treated BMDMs indicates that M1-macrophage associated cytokines are enhanced upon exposure to low doses of PEG-C′ dots. Accordingly, low dosages of C′ dots are an effective means of inducing a pro-inflammatory tumor microenvironment.

The cytokine expression of each cytokine of FIGS. 5A-F was assessed using a Luminex® multiplexed cytokine analysis. The analysis was performed on supernatant collected from BMDM/PEG-C′ dot co-cultures after BMDMs had been exposed to either 5 nM or 100 nM PEG-C′ dots for 6 h, 24 h, 48 h, 1 week, or 2 weeks.

PEG-C′ dot-exposed BMDMs demonstrate significant increases in M1 macrophage-related cytokines TNFα (FIG. 5A), IL-12p40 (FIG. 5B), and IL-12p70 (FIG. 5C) with time and the presence of C′ dots. IL-12p40 and IL-12p70 are also cytokine markers of T-cell activation. The release profiles of M2 macrophage-related cytokines IL-4 (FIG. 5D), IL-10 (FIG. 5E), and IL-13 (FIG. 5F) were also monitored. IL-13 expression can be seen diminishing with time the cells were exposed to C′ dots.

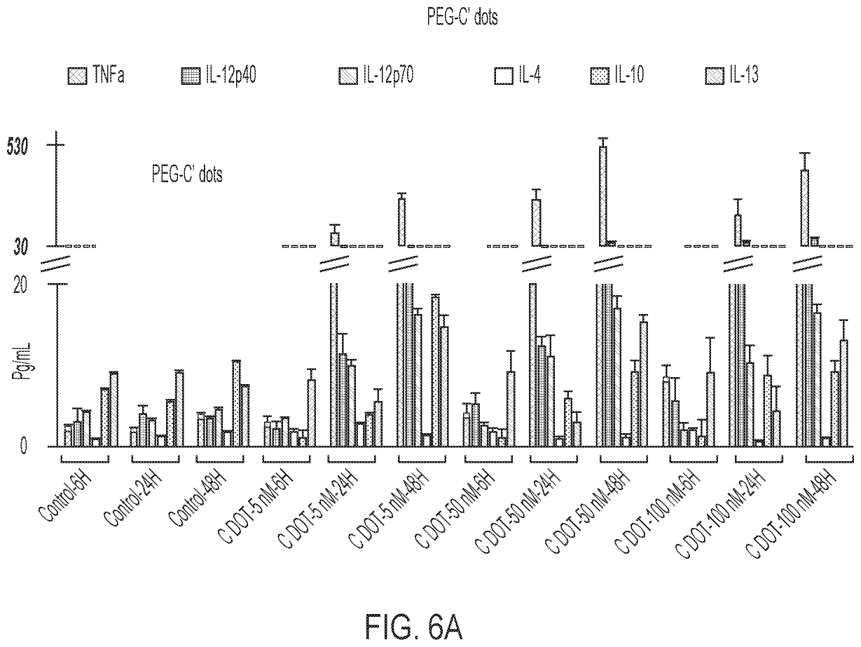

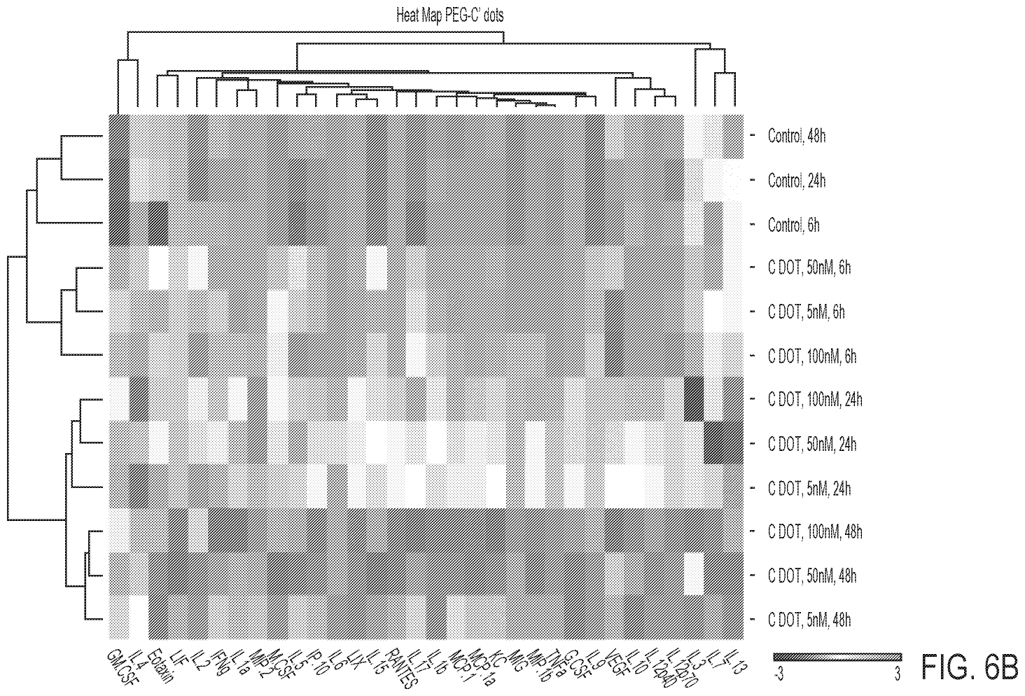

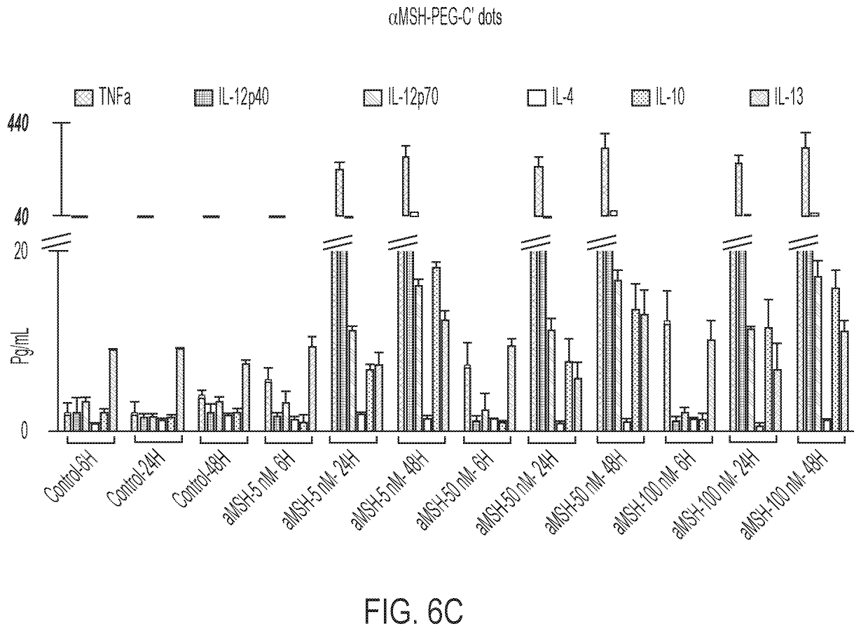

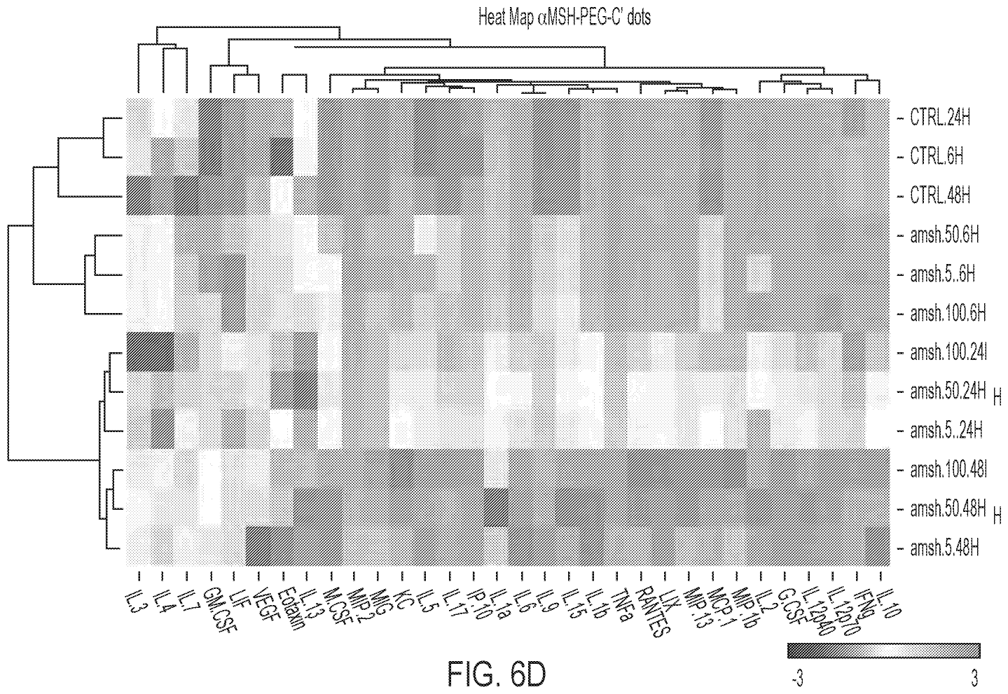

Furthermore, the cytokine profiles and heat maps of PEG-C′ dot-exposed BMDMs (FIGS. 6A-B) were compared with cytokine profiles and heat maps of αMSH-PEG-C′ dot-exposed BMDMs (FIGS. 6C-D). αMSH-PEG-C′ dots target the melanocortin-1 receptor. Without wishing to be bound to any particular theory, the MC1-receptor aids in uptake into normal murine BMDMs. However, significant differences between the particle types were not observed. The amount of cytokines released into the supernatant (FIG. 6A and FIG. 6C) in both particle types are seen to increase with both C′ dot-concentration and time cells were exposed to the C′ dots. Furthermore, heat maps created by looking at the cytokine secretion profiles of the C′ dot treated cells (FIG. 6B and FIG. 6D) also indicate that both kinds of C′-dots induce increased cytokine expression with prolonged exposure and when cells are exposed at increased concentrations.

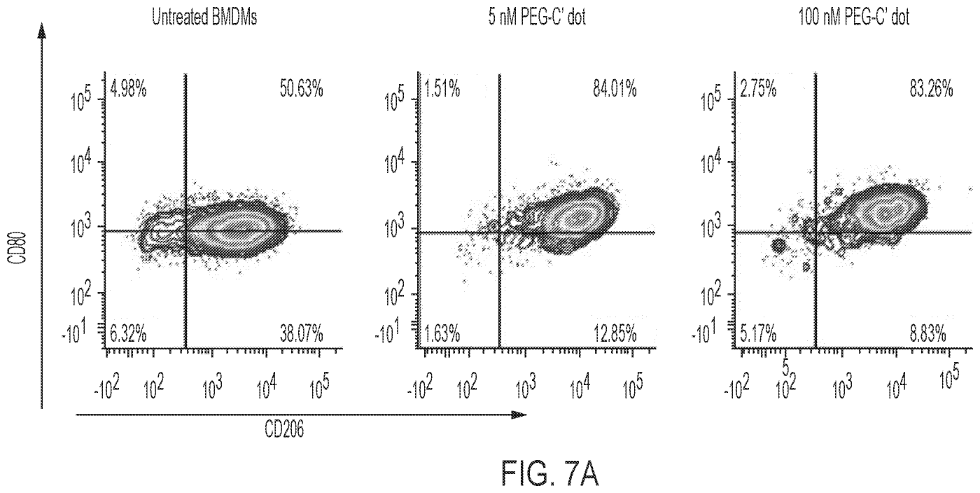

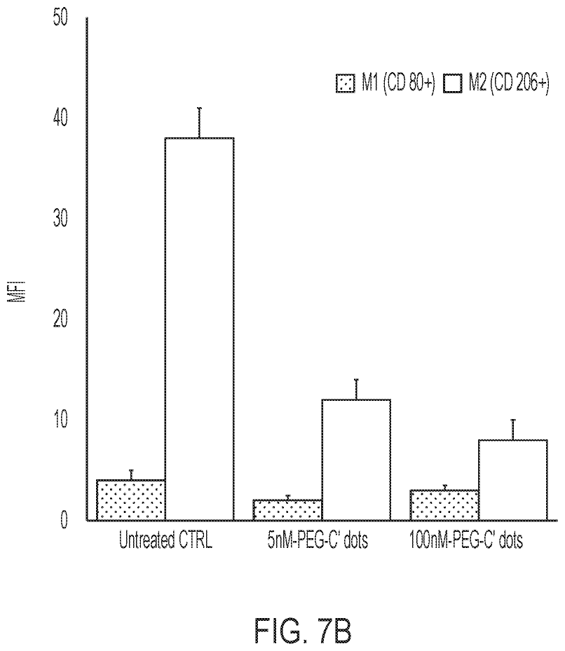

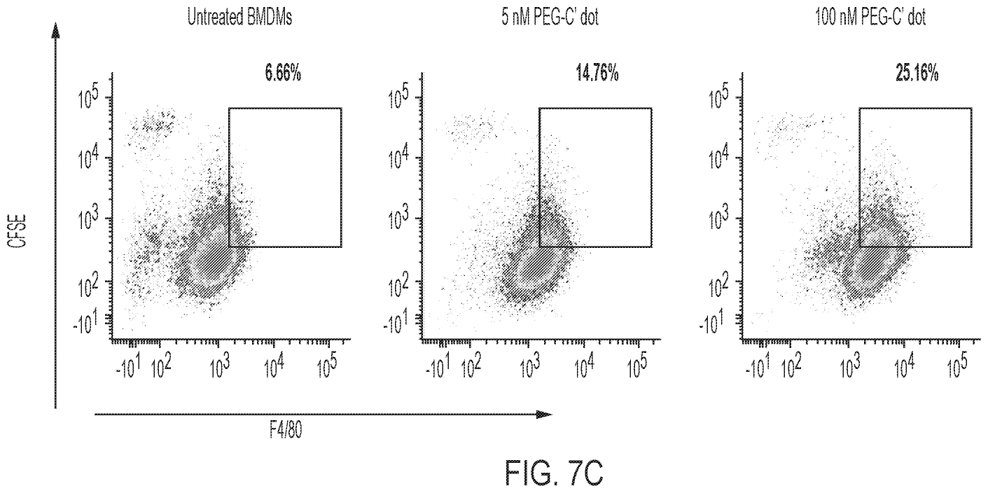

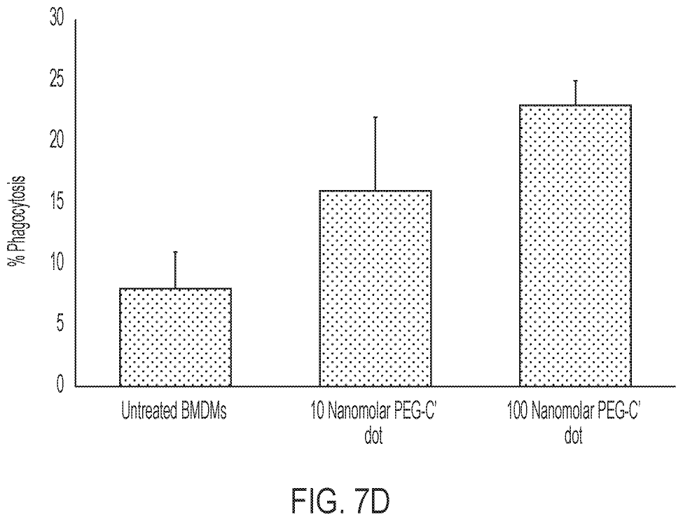

In addition, FIGS. 7A-B shows the effects of PEG-C′ dots on BMDM polarization. BMDMs were cocultured with either 0, 5 nM, or 100 nM PEG-C′ dots for the duration of the experiment. FIG. 7A shows a representative flow cytometry plot for BMDM polarization along with a plot (FIG. 7B) of the mean fluorescent intensity (MFI) of M1 (CD80) and M2 (CD206) phenotype markers. The flow cytometry results and plot indicate a progressive, significant decrease in the M2 phenotypic marker, CD206+ with increasing concentration of PEG-C′ dots.

FIGS. 7C-D shows differential rate of phagocytosis for murine BMDM (F4/80) towards CFSE-expressing GBM (glioblastoma) cells. FIG. 7C is a representative flow cytometry plot of cell populations labeled with the two markers. The rate at which tumor cells are phagocytosed is enhanced through exposure of GBM-BMDM co-cultures exposed to PEG-C′ dots at either 5 nM and 100 nM concentrations (see FIG. 7D). Accordingly, the data shows that treatment with C′ dots enhances the ability of BMDMs to phagocytose tumor cells in the tumor microenvironment.

### Example 5: C′ Dot Inhibition of Mouse Model of Glioblastoma

In certain embodiments, nanoparticle (e.g., C′ dot) administration also inhibits the growth of PDGF-B-driven genetically-engineered mouse model of glioblastoma. Multiple doses of αMSH-C′ dots (36 nmoles total administered from a 60 μM stock in saline) i.v.-injected into a mouse model of GBM led to a reduced percentage of pro-tumor macrophages (e.g., TAMs, M2 macrophages) within glioma.

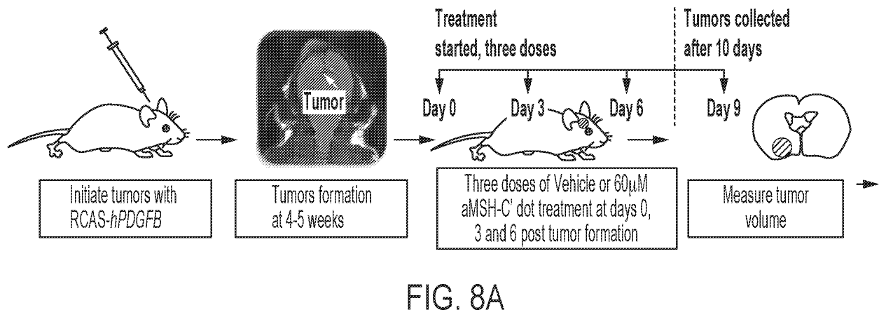

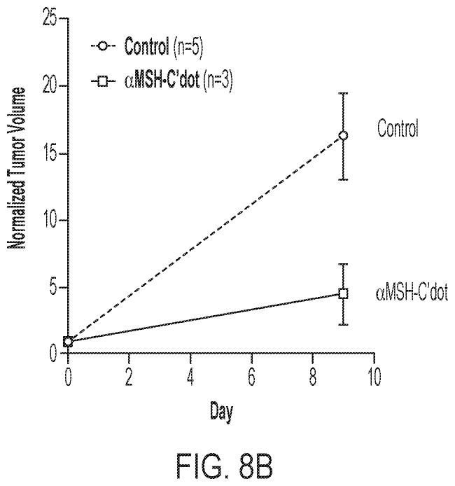

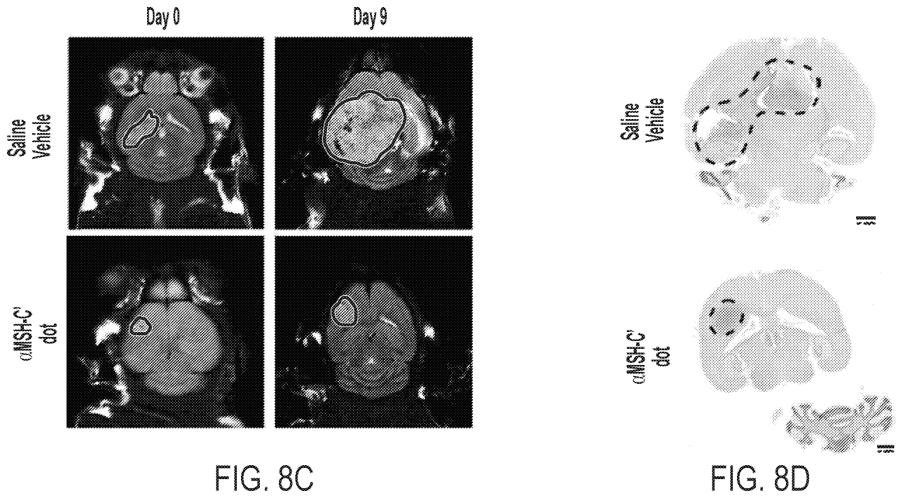

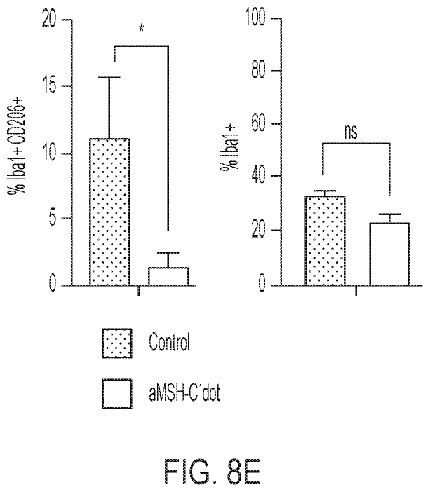

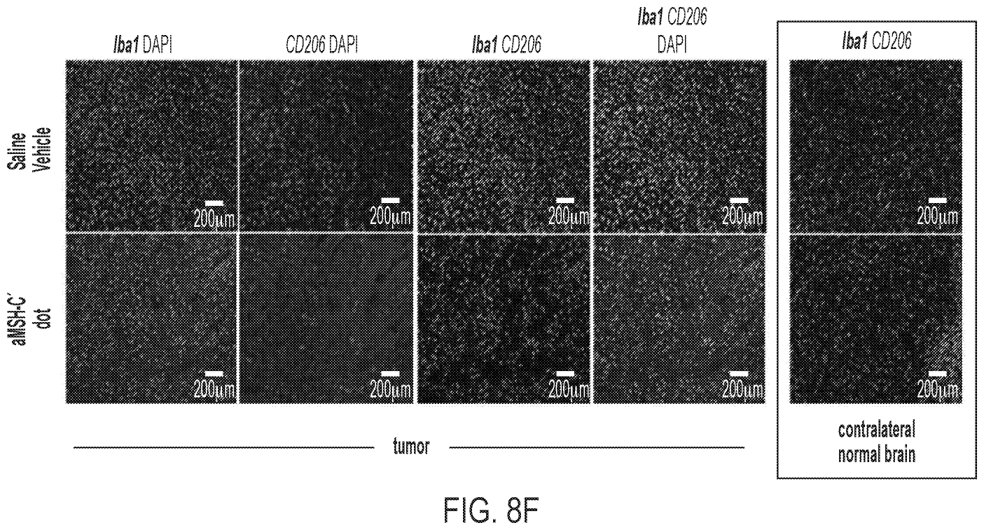

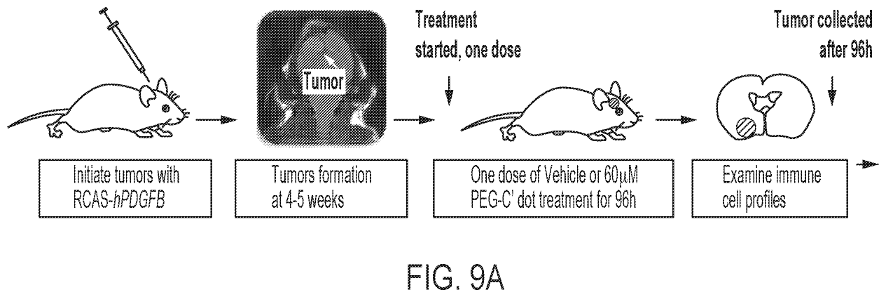

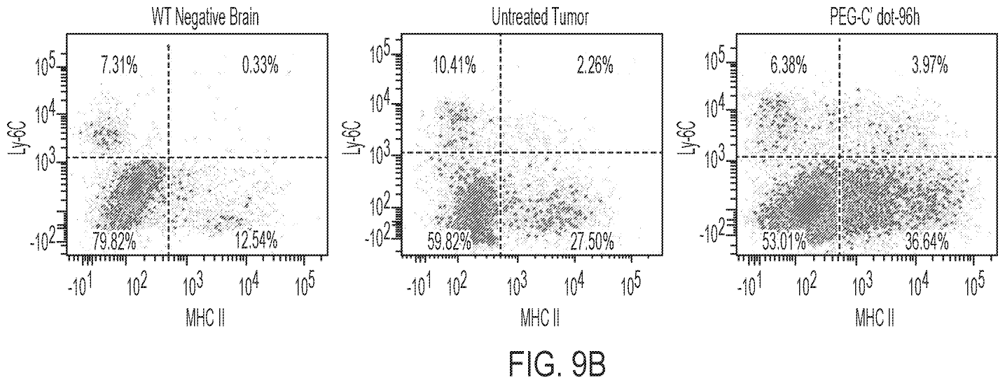

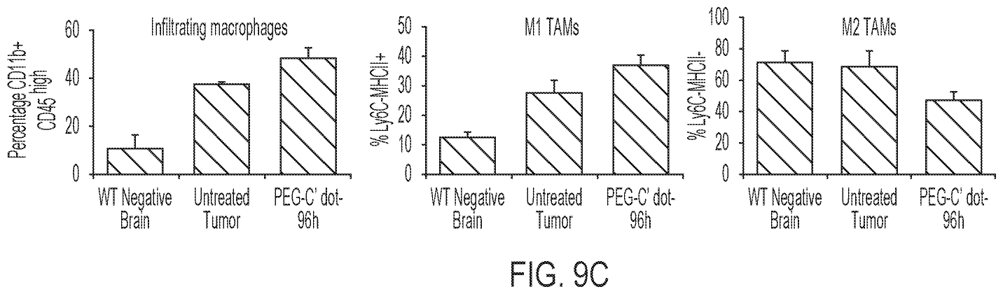

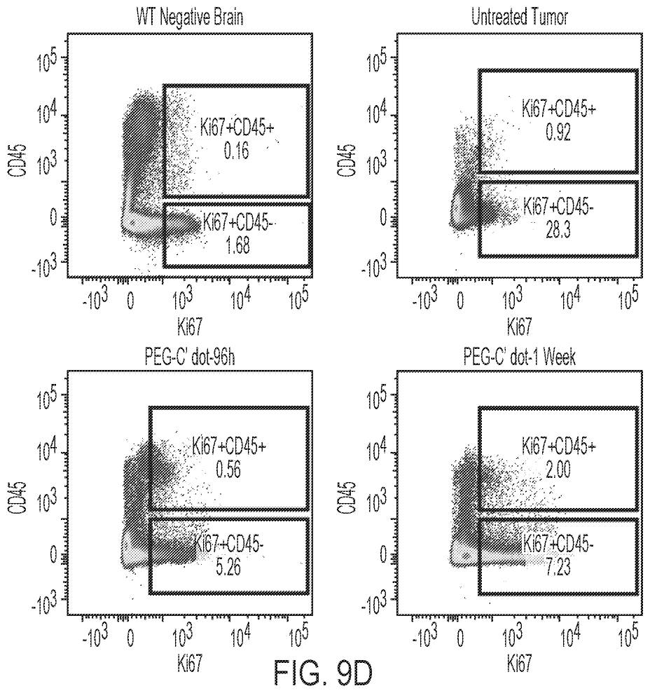

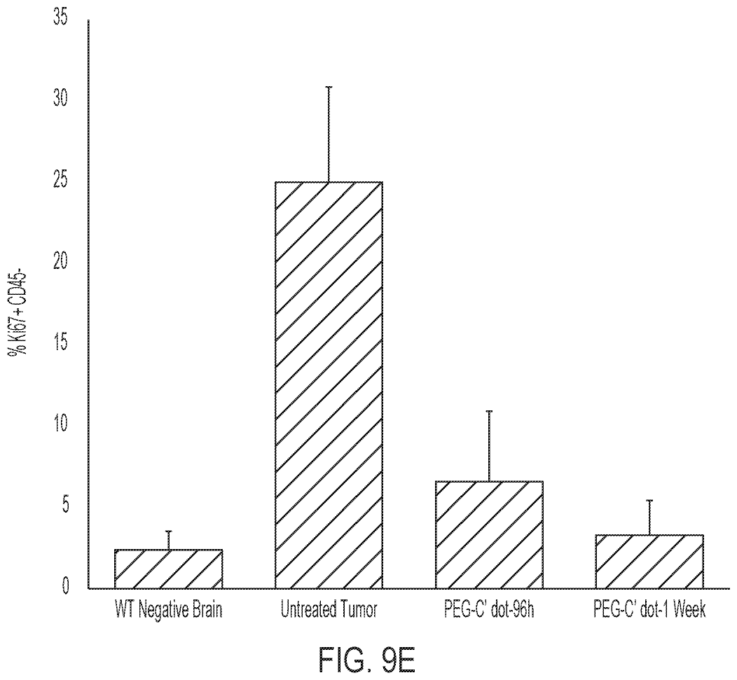

PDGFB-driven high grade gliomas in mice were initiated by stereotactic injection of retrovirus producing DF-1 cells into the brains of adult Nestin-tv-a Ink4a-Arf-/-mice (FIG. 9A). When tumors were identified on magnetic resonance imaging (MRI) at 4-5 weeks after initiation, mice were treated, as indicated in FIG. 8A, with αMSH-C′ dots on days 0, 3 and 6. Brains were harvested after 9 days. Normalized tumor volume measurements (FIG. 8B) were performed in mice i.v.-injected with either saline vehicle control (n=5; circle) or having been administered three doses of 36 nmoles (60 μM stock) of αMSH-C′ dots (n=3; square) using small animal MRI. As can be seen from the graph, normalized tumor volume increases much more with time in mice treated with saline vehicle alone as compared with mice administered αMSH-C′ dots.

Furthermore, similar results can be seen when comparing MRI images and H&E stained brains from mice having tumors. FIG. 8C shows corresponding coronal MR images comparing tumor growth in a mouse administered saline vehicle (top row) and in a mouse administered αMSH-C′ dots (bottom row) at days 0 and 9 of the experimental procedure as outlined in FIG. 8A. As can be seen in FIG. 8C, the tumor in the mouse treated with αMSH-C′ dots is much smaller at 9 days than the tumor in the mouse having been administered saline vehicle alone. Similarly, FIG. 8D shows a H&E (hematoxylin and eosin) staining of tumors, outlined in a dashed line, in the brain of a mouse having been administered saline vehicle (top panel) and in the brain of a mouse having been administered αMSH-C′ dots (bottom panel). As can be seen from the images, growth of the tumor has been significantly slowed and/or prevented by the administration of αMSH-C′ dots.

To demonstrate the effect of αMSH-C′ dots on the tumor microenvironment, the macrophage populations were quantified to determine the effect of αMSH-C′ dots on M2 macrophage polarization. M2 macrophages can be identified by finding cells which are both Iba1 and CD206 positive using, for example, immunofluorescence. The number of macrophages in an image may be quantified by identifying those cells which are Iba1 positive. FIG. 8E shows a graph indicating that the percentage of M2 polarized macrophages in the tumor microenvironment of the brain decreases with the administration of αMSH-C′ dots as compared to the control. In addition, the overall percentage of macrophages of the imaged regions does not significantly change. FIG. 8F shows representative immunofluorescent images from brain tumors and contralateral normal brain from mice that have been administered either αMSH-C′ dots (bottom row of panels) or saline vehicle (top row of panels).

These findings were confirmed in a separate study (FIG. 9A-D) of a mouse model of glioblastoma (FIG. 9A). After 4-5 weeks of tumor formation, the mouse having a PDGFB-driven high grade glioma was treated with either a single low-dose of PEG-C′ dots (12 nmoles of a 60 μM stock PEG-C′ dot solution) or a saline vehicle (i.e., control). At 96 hours post intravenous delivery of the particles or vehicle, M1-like (MHC-II^{higher}Ly6C^{low}) tumor-associated macrophages increased in PEG-C′ dot-treated tumors relative to vehicle-treated and wild-type (WT) tumors, while M2-like (MHC-^{II-}Ly6C^{low}) macrophages decreased relative to controls (FIG. 9B and FIG. 9C).

Moreover, PEG-C′ dots were found to inhibit the proliferation of PDGFB-driven high grade glioma using flow cytometry (FIG. 9D). The relative number of CD45-Ki67+ cells within the brain was significantly reduced in PEG-C′ dot-treated mice compared with untreated control tumors (FIG. 9E). PEG-C′ dot treatment enhances pro-inflammatory responses in high grade glioma. PEG-C′ dot treatment increased proinflammatory responses in brain tumor specimens over 96 h, as well as decreased the anti-inflammatory response of cancer cells in the brain. Accordingly, the relative number of CD45-Ki67+ cells (i.e., non-myeloid cell populations) within the brain was significantly reduced in PEG-C′ dot -treated mice compared with untreated control tumors.

The results presented herein demonstrate that administering C′ dots results in enhancing pro-inflammatory responses and decreasing anti-inflammatory responses in the tumor microenvironment through regulation of populations of macrophages and T cells.

### Example 6: Gene Expression Profiling PDGF-B Driven High Grade Glioma Tumor Specimens

The gene expression profile of ex-vivo tissues were compared to determine the effect of αMSH-C′ dots on tissue bearing PDGFB-driven high grade gliomas.

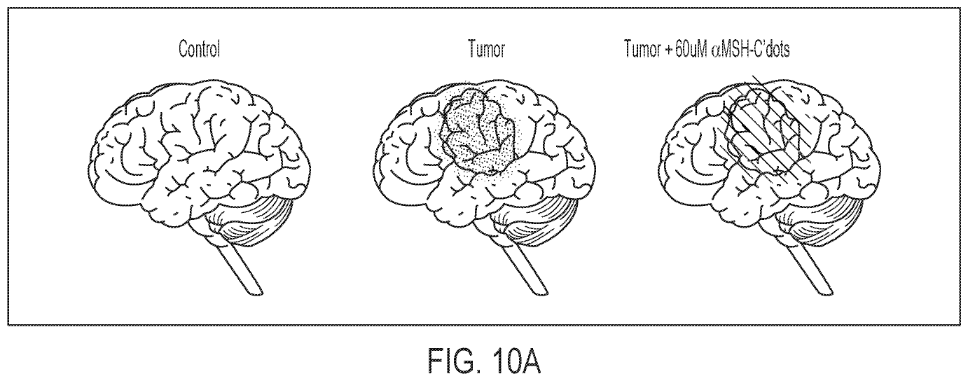

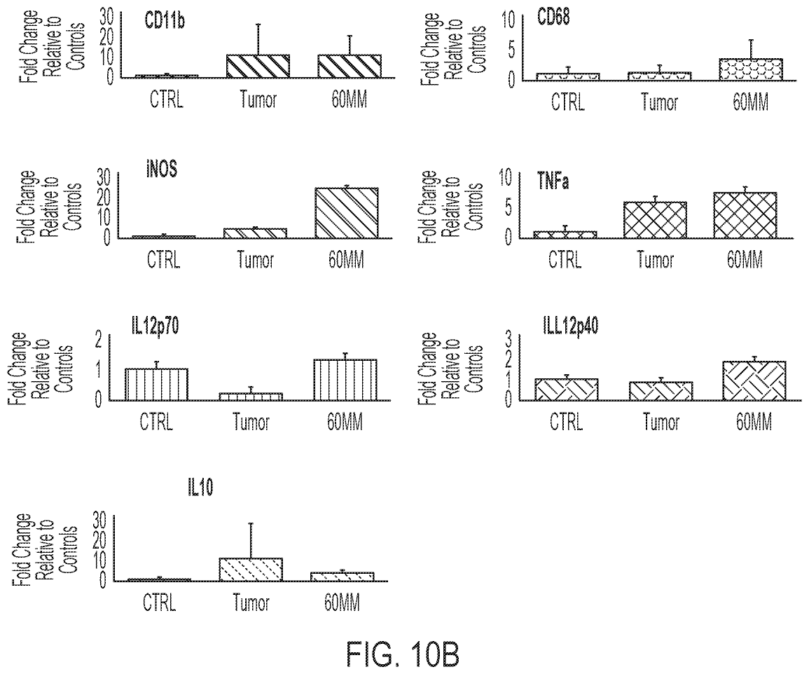

FIG. 10A-B shows results of in vivo studies of gene expression profiling PDGF-driven high grade glioma tumor specimens. FIG. 10A shows the three different conditions corresponding to the treatments. Gene expression profiles (FIG. 10B) were obtained from tissues from brain samples from mice without tumors, brain samples from mice with tumors, and brain samples from mice with tumors treated αMSH-C′ dots. The treated mice were treated with a single intravenous injection of 60 μM of αMSH-C′ dots. The results show upregulation of M1 phenotypic marker expression in tumor tissues treated with αMSH-C′ dots.

### Example 7: Detection of Secreted Cytokines in Whole Tumor-Specimens In Vivo

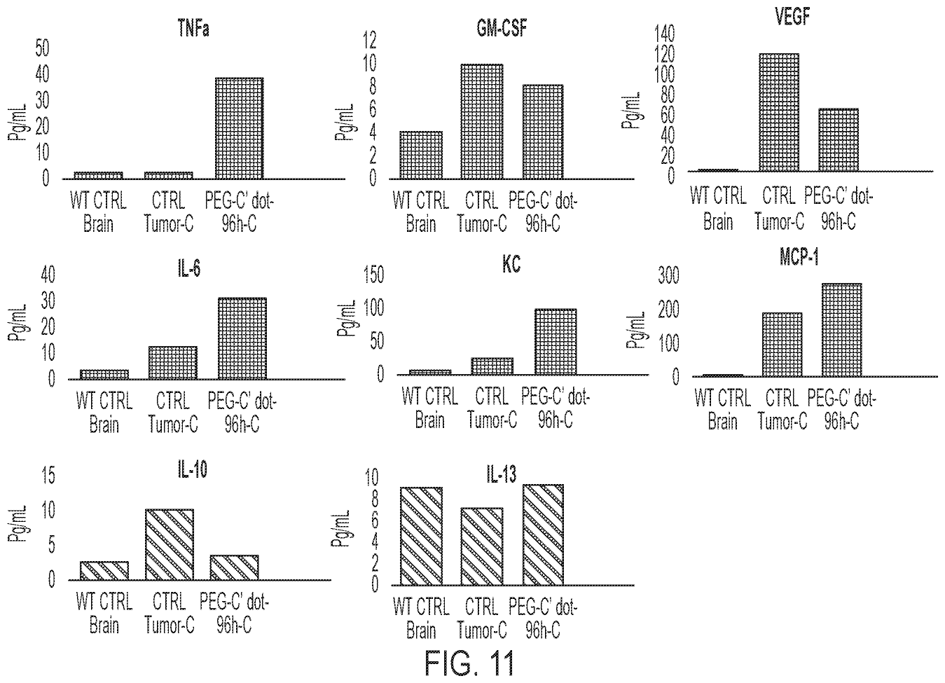

The cytokine release profiles of mice bearing PDGF-B high grade gliomas can be seen in FIG. 11. FIG. 11 shows a separate series of in vivo studies involving detection of secreted cytokines in whole tumor specimens, 96 h post-intravenous delivery of PEG-C′ dots. Exposure of brain tumor specimens bearing tumor to PEG-C′ dots enhances pro-inflammatory responses (e.g., TNFα, MCP-1) for different immune cell populations (e.g., macrophages, T cells, dendritic cells) in the tumor microenvironment but does not promote anti-inflammatory responses (e.g., IL-10, IL-13).

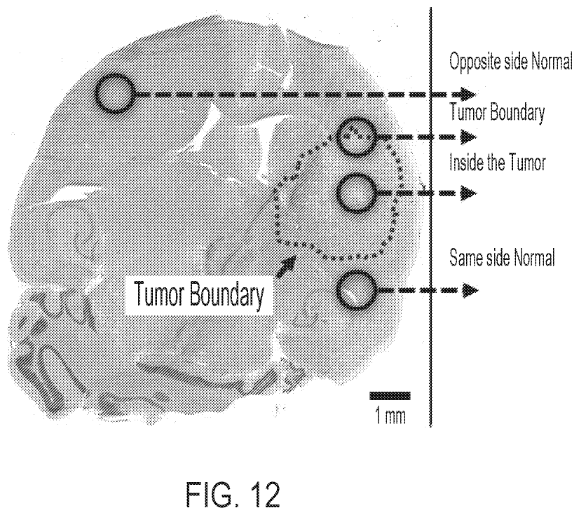

Furthermore, in vivo studies of immunotherapeutic modulation of PDGF-driven high grade gliomas and the surrounding brain parenchyma were studied through quantifying the release of cytokines and chemokines. A single intravenous injection of 60 μM αMSH-PEG-C′ dots (n=3), 60 μM PEG-C′ dots (n=3) or saline was administered to PDGF-B tumor bearing mice at an initial time point. Control mice (n=3) not bearing tumors and having been administered saline at an initial time point were also included. The brains of mice were extracted after 96h. Tumor and brain samples were processed into single-cell suspensions by manual dissociation. Cell supernatant was collected and analyzed for cytokines and chemokines using the Luminex assay. Cytokine expression profiles were obtained from regions of the mouse brain as shown in the representative illustration in FIG. 12.

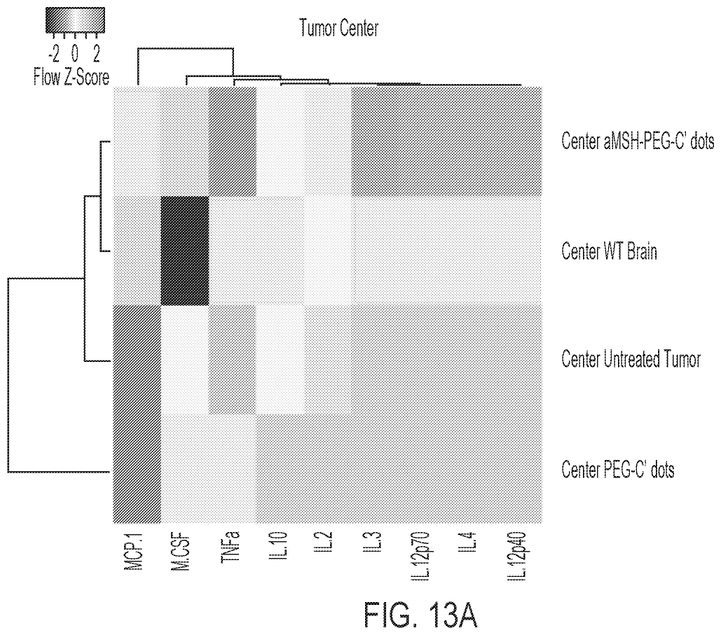

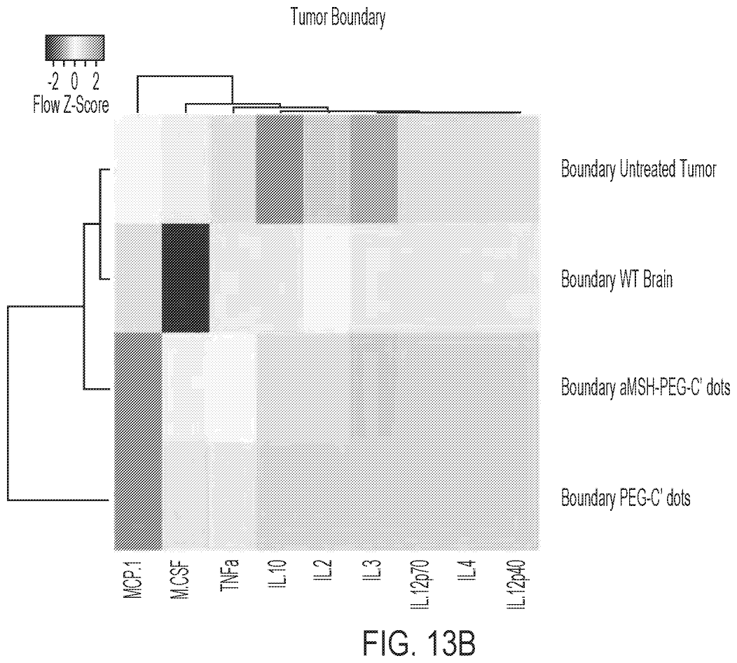

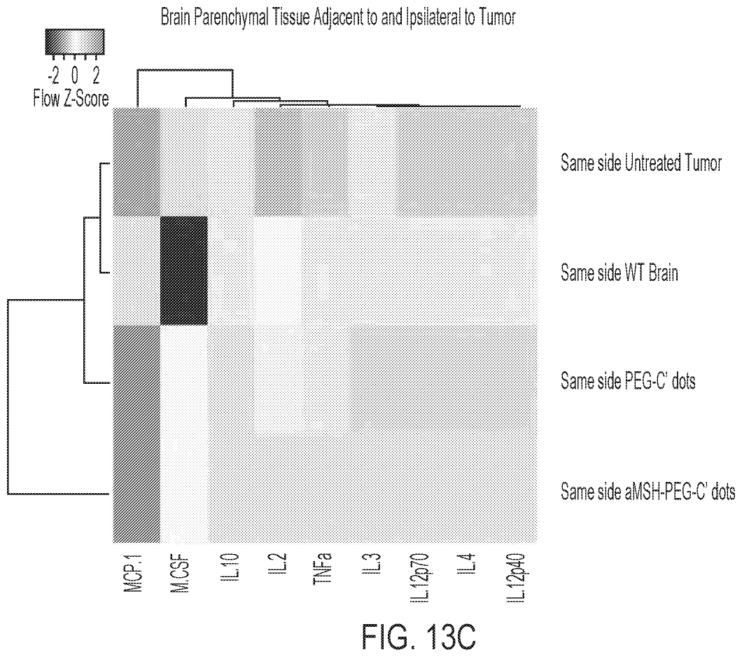

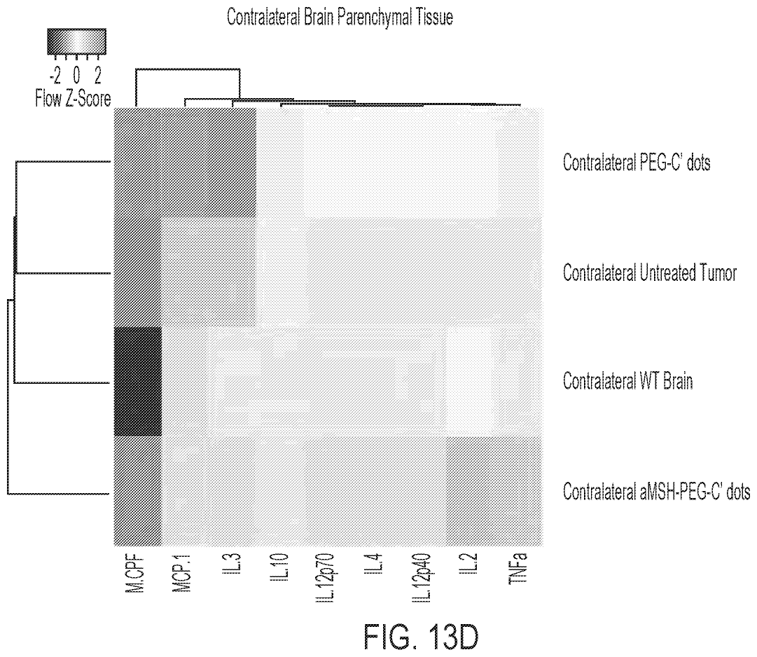

FIGS. 13A-D show heat maps of the cytokine and chemokine release profiles obtained from various locations (e.g., see FIG. 12) within the brain of the mouse. The release profiles demonstrated signs of pro-inflammatory response in the tumor and brain samples of mice administered either type of C′ dots over 96 hrs.

### Example 8: Effect of C′ Dots on T Cell Priming

Furthermore, T cell priming was observed in an in vitro study when cells were treated with either αMSH-C′ dots or PEG-C′ dots at different dosages. CFSE-labeled (Carboxyfluorescein succinimidyl ester-labeled) CD8+μmel-1 T cells expressing gp100 (a melanoma-associated antigen) were co-cultured with C′ dot-exposed bone marrow-derived antigen presenting cells (BM-APCs) loaded with gp100 (FIG. 10A).

The BM-APCs were either exposed to 5 nM or 100 nM of αMSH-PEG-C′ dots or PEG-C′ dots. ‘5c’ indicates 5 nM PEG-C′ dot exposure, ‘100c’ indicates 100 nM PEG-C′ dot exposure, ‘5a’ indicates 5 nM αMSH-PEG-C′ dot exposure, and ‘100a’ indicates 5 nM αMSH-PEG-C′ dot exposure. The first two bars in each graph are indicative of a negative control and a positive control. In the positive control, T cells have been activated with particles covalently coupled with CD3 and CD28 antibodies. T cells in this study were derived from the pmel-1 mouse model.

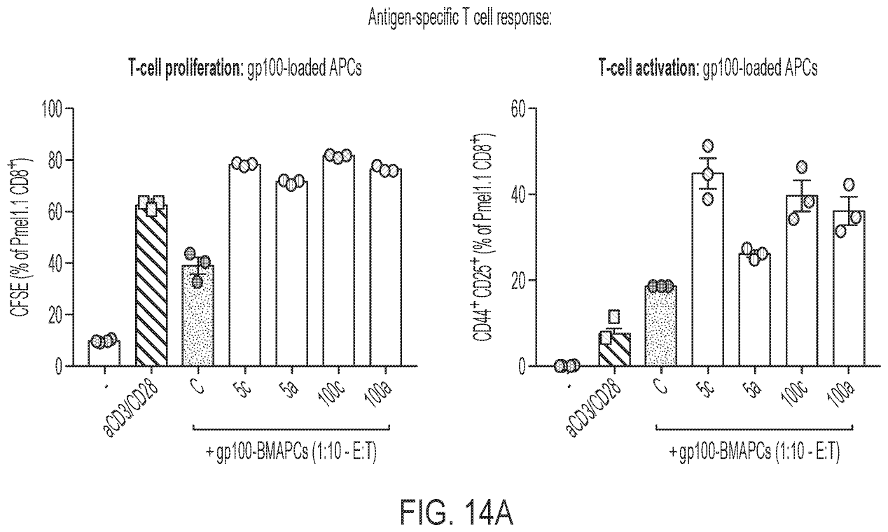

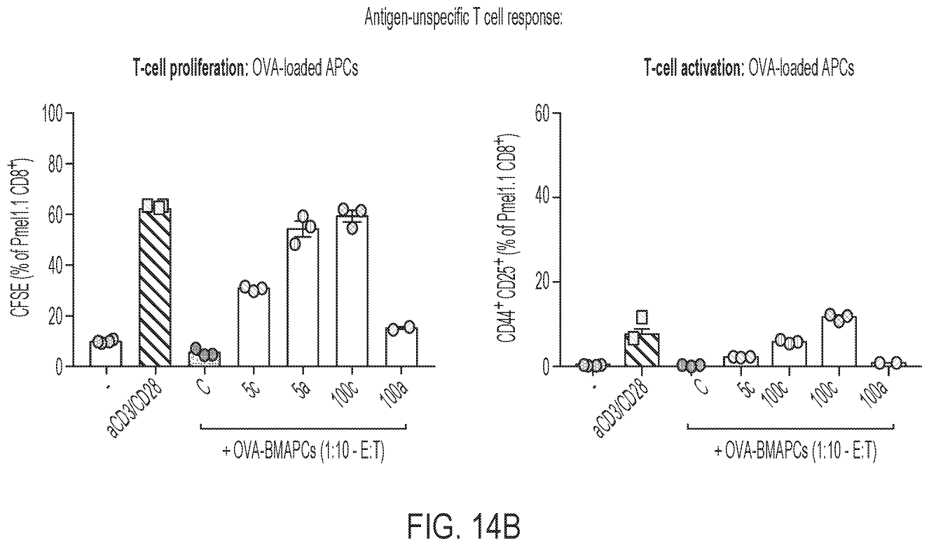

The results in the left panel of FIG. 14A show a significant increase in proliferation rate when T cells are co-cultured with BM-APCs exposed to either αMSH-PEG-C′ dots or PEG-C′ dots as indicated by an increase in CFSE. Furthermore, T-cells show an increase in activation state as indicated by an increase in cells positive for CD44 and CD25 as compared to controls (e.g., as seen in the right panel of FIG. 14A).

For comparison, CFSE-labeled CD8+μmel-1 T cells expressing ovalbumin (OVA) and bone marrow-derived antigen presenting cells (BM-APCs) loaded with OVA were also used in experiments. Using the same experimental conditions as above, the results of this series of experiments are presented in FIG. 14B.

As can be seen by comparing the panels of FIG. 14A and FIG. 14B, FIGS. 14A-B demonstrate that the key finding that T-cell response is antigen-specific.

### Example 9: Human Dendritic Cell Activation

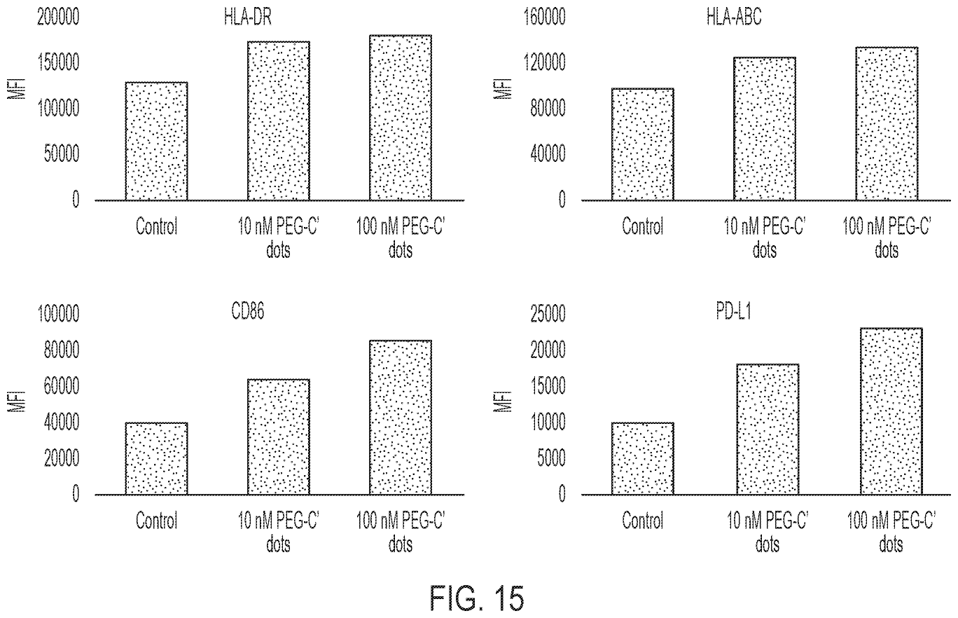

FIG. 15 shows results of in vitro human dendritic cell activation studies carried out using flow cytometry. Markers of MEW class I and class II activation are indicated by HLA-ABC and HLA-DR. The enhancement of CD86 and PD-L1 as seen in FIG. 15 is also key to checkpoint blockade therapy. Accordingly, the experiments demonstrate that treatment of human dendritic cells with PEG-C′ dots activates them and improves their effector functions.

### Example 10: A Genomic Profile of Local Immunity in the Melanoma Microenvironment Following Treatment with Alpha Particle-Emitting Ultrasmall Silica Nanoparticles

In an embodiment of the technology, the technology is directed to nanoparticles targeted to tumor. In certain embodiments, the nanoparticles used are or comprise alpha particle-emitting agents. Nanoparticles as described herein are potent and specific anti-tumor agents and prompt significant remodeling of local immunity (e.g., the populations of immune cells, the activation and/or polarization status of immune cells) in the tumor microenvironment.

In certain embodiments, nanoparticles as described herein comprise biocompatible ultrasmall fluorescent core-shell silica nanoparticles (e.g., fluorescent C′ dots). Nanoparticles have been engineered to target the melanocortin-1 receptor (MC1-R) expressed on melanoma cells via the conjugation of alpha melanocyte stimulating hormone (αMSH) peptides to the C′ dot surface. In certain embodiments, one or more isotopes (e.g., Actinium-225) are also bound to the C′ dot to deliver a densely ionizing dose of high-energy alpha particles to cancer. Pharmacokinetic properties of the C′ dot are optimal for targeted radionuclide therapy as C′ dots exhibit rapid blood clearance, tumor-specific accumulation, minimal off-target localization, and are renally eliminated. Potent and specific tumor control, arising from the alpha particles, is observed in, for example, syngeneic animal models of melanoma.

Surprisingly, the C′ dot component initiates a favorable pseudo-pathogenic response in the tumor microenvironment. The C′ dot generates distinct changes in the fractions of naive and activated CD8 T cells, Th1 and regulatory T cells, immature dendritic cells, monocytes, MΦ and M1 macrophages, and activated natural killer cells. Concomitant upregulation of the inflammatory cytokine genome and adaptive immune pathways each describes a macrophage-initiated pseudo-response to a viral-shaped pathogen. Accordingly, therapeutic alpha-particle irradiation of melanoma using ultrasmall functionalized core-shell silica nanoparticles (i.e., C′ dots) potently kills tumor cells, and initiates a distinct immune response in the tumor microenvironment.

### Introduction

In certain embodiments, alpha melanocyte stimulating hormone (αMSH) analog peptide sequences designed to target the melanocortin-1 receptor (MC1-R) expressed on melanoma are attached to the surface of nanoparticles. In certain embodiments, another synthetic modification includes covalent attachment of chelating agents pre-loaded with alpha particle-emitting radionuclide (e.g., Actinium-225) in order to deliver a cytotoxic dose of radiation to the tumor. Actinium-225 (^{225Ac; t}1/2=10 days) deposits a high dose of energy (5-8 MeV) over a short range (50-80 μm), producing specific and potent cytotoxicity. High-linear energy transfer (LET) alpha particles are lethal to cancer cells as a consequence of ineffective double-strand DNA repair. Moreover, in internalizing systems such as αMSH-PEG-Cy5-C′ dot, each ²²⁵Ac decay produces several daughters, which generate three additional alpha particles able to contribute to cytotoxicity.

Malignant melanoma is diagnosed in approximately 90,000 individuals in the United States per year and is the most lethal form of skin cancer. The incidence of disease has increased rapidly over the past 50 years. Melanoma is an aggressive disease and metastatic stage-IV melanoma is difficult to treat despite advances in immunotherapies. Median survival of subjects diagnosed with stage-IV melanoma ranges from 8 to 12 months with standard-of-care treatment including immunotherapeutic drugs such as ipilimumab and nivolumab. Nanomolecular drug agents constructed from silica permit MC1-R targeting and allow selective delivery of the alpha-particle emitters (e.g., Actinium-225), yielding a potent new treatment option for metastatic melanoma. Close inspection of the tumor microenvironment (TME) following irradiation of disease indicates that the immune cell composition, cytokine mRNA, and inflammatory pathways undergo dynamic changes arising from the use of the C′ dot platform.

Radiotherapy upregulates cytokine signaling and inflammatory cascades and nanomaterials have been recognized as contributing factors in modifying the immune milieu. Herein, the pharmacology of the radiotherapeutic alpha particle-emitting [²²⁵Ac]αMSH-PEG-Cy5-C′ dot drug is described. Also described herein is the unexpected contribution of the αMSH-PEG-Cy5-C′ dot nanoparticle platform in an immunocompetent, syngeneic mouse model of melanoma. The study discussed herein describes downstream effects on TME immune cell populations, cytokine expression and inflammatory pathways arising from an alpha particle-emitting ultrasmall fluorescent core-shell silica nanoparticle and explores the unique combination of alpha particles and C′ dot-based adjuvant immunotherapeutic approaches to eliminate melanoma.

### Chemical Synthesis and Physical Characterization of Ultrasmall Alpha Melanocyte Stimulating Hormone (αMSH)-Functionalized C′ Dot Precursors

In certain embodiments, the nanoparticles described herein are synthesized as follows.

Precursor αMSH-PEG-Cy5-C′ dots were synthesized as follows: Heterobifunctional N-hydroxysuccinimide ester polyethylene glycol maleimide (NHS-PEG-Mal, Quanta Biodesign; 860 g/mol, 12 ethylene glycol units per molecule) was reacted at ambient temperature (e.g., about 20° C.) with (3-aminopropyl)triethoxysilane (APETS, Sigma Aldrich) under nitrogen to form Mal-PEG-silane. The αMSH peptide was subsequently added to the Mal-PEG-silane at ambient temperature under nitrogen to produce αMSH-PEG-silane.

The Cy5-silane component was prepared by conjugating maleimido-functionalized Cy5 dyes (GE Healthcare Life Sciences) with (3-mercaptopropyl) trimethoxysilane (APTMP, Sigma Aldrich) at ambient temperature under nitrogen. Tetramethyl orthosilicate (TMOS, Sigma Aldrich) and Cy5-silane were then mixed in an aqueous ammonium hydroxide solution (pH adjusted to 8.5) at ambient temperature with stirring. αMSH-PEG-silane and monofunctional PEG-silane (Gelest; approximately 500 g/mol, 6-9 ethylene glycol units) were added into the reaction at ambient temperature with stirring overnight.

The resulting αMSH-PEG-Cy5-C′ dots were dialyzed against deionized water, purified using gel permeation chromatography (GPC), and filtered by sterile syringe filters. The final product was characterized and stored at 4° C. GPC purification and characterization of the synthesized αMSH-PEG-Cy5-C′ dots was conducted using a Biologic LP system (Bio-Rad) equipped with a 275-nm UV detector and a chromatography column packed with Superdex 200 resin (GE Healthcare Life Sciences). Fluorescence correlation spectroscopy (FCS) measurements were conducted using a custom-built FCS instrument with a 633-nm solid-state laser as the excitation source.

Internalization of αMSH-PEG-Cy5-C′ dots by macrophages and B16-F10 tumor cells measured using FACS

FACS Study 1: C57BL/6J mice (Female; 8-12 weeks old; Jackson Laboratory) were implanted with 5×10⁵ B16-F10 cells via subcutaneous (SC) injection and separated into two groups. Each animal received an intravenous (IV) injection of 50 pmole of αMSH-PEG-Cy5-C′ dots formulated in 1% human serum albumin (HSA, Swiss Red Cross)/0.9% NaCl (Abbott Laboratories) (1% HSA) or only the 1% HSA vehicle (0 μmole C′ dots) via retroorbital sinus injection under anesthesia 8 days after B16-F10 implantation. The mice were euthanized 4-5 days post administration of C′ dots. Tumor was harvested and dissociated into single-cell suspensions using using the Tumor Cell Isolation Kit (Miltenyi Biotec, catalog #130-096-730) for 45 minutes at 37° C. with shaking. The single-cell suspensions were individually passed through a 70 μm strainer to isolate single cells, pelleted and resuspended in RPMI media. CD45 Microbeads (Miltenyi Biotec, catalog #130-052-301) were added to separate CD45-positive (CD45⁺) cells from the suspension. The CD45⁺ and CD45⁻ populations were subsequently analyzed by FACS (LSR Fortessa, BD Biosciences) to measure C′ dot internalization by tumor macrophages and melanoma cells.

FACS Study 2: Naive, immunocompetent C57BL/6J mice (Female; 8-12 weeks old; Jackson Laboratory) each received an intraperitoneal (IP) injection 50 pmole of αMSH-PEG-Cy5-C′ dots in 1% HSA or vehicle alone. Macrophages were isolated from the IP cavity at 48 hours and analyzed by FACS to measure C′ dot internalization.

In both in vivo studies, the harvested cells were blocked with mouse FcR Blocking Reagent (Miltenyi Biotec). Macrophages were stained with PE/Cy7 anti-mouse F4/80 (BioLegend, #123113). B16-F10 tumor cells were stained with PE/Cy7 anti-mouse Podoplanin (PDPN, BioLegend, #127411). C′ dots exhibit Cy5 fluorescence. All cells were stained with 1 g/L 4′,6-diamidino-2-phenylindole (DAPI, Sigma Aldrich). Compensation controls were performed using single-color staining of cells or UltraComp eBeads compensation beads (ThermoFischer Scientific).

FACS Study 3: Wild type THP-1 cells (THP-1^{wt}), phorbol 12-myristate 13-acetate (PMA, Sigma-Aldrich) differentiated THP-lcells (THP-1PMA), and B16-F10 cells were treated with 25 μmole of αMSH-PEG-Cy5-C′ dots in PBS or only the PBS vehicle (0 μmole C′ dots). Cells were analyzed by FACS to measure C′ dot internalization at 20, 48, 72, and 96 hours. All data were acquired with the LSR Fortessa using FACSDIVA software (version 8.0.1, BD Biosciences) and analyzed using FlowJo software (version 10.5.3 for Mac, Tree Star Inc). Debris and doublets were excluded using light scatter measurements. Dead cells were excluded by DAPI staining.

### ²²⁵Actinium Radiochemical Labeling of C′ Dots and Quality Control

In certain embodiments, a two-step radiochemical labeling methodology is employed to prepare ²²⁵Ac-labeled αMSH-PEG-Cy5-C′ dots.

37 MBq (1 mCi) of acidic ²²⁵Ac nitrate (U.S. Department of Energy (ORNL, TN)) dissolved in 0.2 M HCL (hydrochloric acid; Fisher Scientific) was added to a solution of 0.5-1.0 mg of S-2-(4-isothiocyanatobenzyl)-1,4,7,10-tetraazacyclododecane tetraacetic acid (DOTA-Bz-SCN, Macrocyclics, Inc.) in 0.10 mL metal-free water. The pH of the reaction mixture was adjusted to about 5.5 through the addition of 0.1 mL of 2M tetramethylammonium acetate (Sigma Aldrich) and 0.02 mL of 150 g/L 1-ascorbic acid (Sigma Aldrich). The reaction mixture was heated to about 55-60° C. for 30 min.

An aqueous solution of the αMSH-PEG-Cy5-C′ dots (3 nmol in 0.225 mL water) was added to the [²²⁵Ac]DOTA-Bz-SCN reaction mixture. The pH of the reaction mixture was adjusted to 9.5 with the addition of 0.15 mL of 1 M carbonate/bicarbonate buffer solution (Fisher Scientific). The reaction was held at about 37° C. for 30-60 min. The reaction within the mixture was subsequently quenched with 0.020 mL of 50 mM diethylenetriaminepentaacetic acid (DTPA, Sigma Aldrich).

The reaction mixture was purified by size exclusion chromatography (SEC) using a P6 resin (BioRad) as the stationary phase and 1% HSA as mobile phase. The radiochemical purity of the final radiolabeled product, [²²⁵Ac]αMSH-PEG-Cy5-C′ dot, was determined by instant thin-layer chromatography using silica gel (ITLC-SG). ²²⁵Ac activity was assayed in a Squibb CRC-15R Radioisotope Calibrator (E.R. Squibb and Sons, Inc.) set at 775. The displayed activity was multiplied by 5 at secular equilibrium. ²²⁵Ac-radiolabeled NH₂-PEG-Cy5-C′ dots used in control experiments were prepared by attaching [²²⁵Ac]DOTA-Bz-SCN to the primary amine groups on the surface of NH₂-PEG-Cy5-C′ dots using an approach referred to as post PEGylation surface modification by insertion (PPSMI). The resulting [²²⁵Ac]NH₂-PEG-Cy5-C′ dots were quality controlled in the same way as presented above for the targeted dots.

### Pharmacokinetic Studies

Tissue biodistribution and clearance studies were performed using an immunocompetent C57BL/6J mouse model (Female; 6-8 weeks old; Jackson Laboratory, Bar Harbor, ME). The tissue distribution, blood compartment clearance and renal excretion of [²²⁵Ac]αMSH-PEG-Cy5-C′ dot were measured in both (a) healthy and (b) B16-F10 tumor-bearing animals.

Tumors were initiated with a subcutaneous (SC) injection of 105 B16-F10 cells. All experiments were done in accordance with the guidelines of the National Institutes of Health on the care and use of laboratory animals and all protocols were approved by the Memorial Sloan Kettering Institutional Animal Care and Use Committee.

Healthy naïve mice received an IV injection of 11.1 kBq (300 nCi) of [²²⁵Ac]αMSH-PEG-Cy5-C′ dots via retroorbital sinus injection under anesthesia (n=3 mice per time point) and were later euthanized. Tissues, blood and urine were harvested at 1, 24, 48, 72, and 144 hours post injection.

Mice with SC melanoma received an IV injection of 11.1 kBq (300 nCi) of [²²⁵Ac]αMSH-PEG-Cy5-C′ dot via retroorbital sinus injection under anesthesia (n=5 per group) and were euthanized with tissues, blood and urine harvested at 1, 24, 96, and 120 hours post injection. The tissue samples were weighed and the ²²⁵AC activity measured at secular equilibrium using a gamma-counter (COBRA II, Packard Instrument Company, Meriden, Conn.). The 370-520 keV energy window was used to quantitate the activity per tissue. Samples of each injectate formulation were used as decay correction standards. Data were expressed as %ID/g. Aliquots of the injected drug (0.020 mL) were used as decay correction standards. The percentage of the injected dose of [²²⁵Ac]αMSH-PEG-Cy5-C′ dot per gram of tissue weight (%ID/g) was calculated for each animal, decay-corrected to the time of injection, and the mean %ID/g was determined at each time-point.

### Absorbed Dose Estimates

The absorbed doses to tissues from [²²⁵Ac]αMSH-PEG-Cy5-C′ dot were estimated from %ID/g values derived from the biodistribution data. For each tissue, the %ID/g values were plotted versus the time post injection and fit to an exponential function. The resulting time-activity functions were then analytically integrated, incorporating the effect of the radioactive decay, to obtain the tissue residence times (MBq-s/MBq administered) of ²²⁵Ac. For each tissue, the absorbed dose (in cGy/MBq of ²²⁵Ac administered) in mice was then calculated by multiplying the tissue residence time concentration (MBq-s/kg) by the ²²⁵Ac equilibrium dose constant for non-penetrating radiations (alpha particles), 9.39×10⁻¹¹ cGy-kg/MBq-s, assuming complete local absorption of the alpha particles and ignoring the very small beta-particle and gamma-ray dose contribution.

The ²²⁵Ac tissue residence times in the 70-kg Reference Man anatomic model were obtained by inverse scaling based on the body masses of the Reference Man and a 25-gram mouse and using the Reference-Man tissue masses. Reference-Man tissue absorbed doses were then calculated using the OLINDA/EXM internal-radionuclide dosimetry computer program.

### Determination of the Maximum Tolerated ²²⁵Ac Dose

Naïve, immunocompetent C57BL/6J mice (female; 6-8 weeks old; Jackson Laboratory) were randomized to four separate groups (n=5 per group). Animals in Groups 1, 2, 3, and 4 each received an IV injection of 0, 23.1, 46.3, or 92.5 kBq of [²²⁵Ac]αMSH-PEG-Cy5-C′ dot, respectively, via retroorbital sinus injection under anesthesia. The animals were monitored regularly to assess overall health and were weighed weekly for 4 weeks. Toxicity was scored when body weight loss was ≥10% compared to baseline or there was severe lethargy or death. Survival data was analyzed by the Kaplan-Meier method using Prism software.

### Pharmacodynamic Studies

Radiotherapeutic alpha particle effects on tumor growth and animal survival were assessed using immunocompetent C57BL/6J mice (female and male; 6-8 weeks old; Jackson Laboratory, Bar Harbor, Me.). Each animal received SC injections of 105 B16-F10 cells. 8 days later the mice were randomly sorted into three groups of 10 animals (5 females and 5 males per group).

Mice in each group received an IV injections as follows:

Group I: 11.1 kBq of [²²⁵Ac]αMSH-PEG-Cy5-C′ dot

Group II: 11.1 kBq of [²²⁵Ac]NH2-PEG-Cy5-C′ dot

Group III: 1% HSA injection vehicle

IV injections were administered via retroorbital sinus injection under anesthesia. The specific activity of the injected C′ dots is 227,484±57,583 GBq/mol (n=6) and 55±12 μmole of C′ dots were injected into each mouse.

Mice were sacrificed when tumor was ≥2,500 mm³ or if they exhibited lethargy. Survival was plotted using the Kaplan-Meier method. Tumor samples from representative animals were harvested for histopathology.

### Immune Cells Populating the Alpha-Irradiated Tumor Microenvironment

Immunofluorescence (IF) staining of tumor tissue harvested from the Group I [225Ac]αMSH-PEG-Cy5-C′ dot-treated mice and Group III vehicle-treated mice (see Pharmacodynamic studies section above) was performed to image the kinetics of immune cell in the tumor microenvironment (TME) post treatment.

Representative animals were euthanized at 1, 24, 96, and 120 hours post treatment. Harvested tumor was fixed in 4% paraformaldehyde/PBS for 24 hours. Fixed tissue was paraffin-embedded and cut into 5-μm sections and mounted for imaging. IF staining was performed at the MSKCC Molecular Cytology Core Facility using a Discovery XT processor (Ventana Medical Systems). Stains used are anti-CD3 (eBioscience, #A0452, 0.5 μg/mL) and anti-IBA1 (Vector, #091-19741, 0.4 μg/mL). Tumor tissue sections were scanned using a Mirax digital slide scanner (Carl Zeiss Microimaging) with a ×20 lens and analyzed with Pannoramic Viewer software.

Transcriptome Sequencing of CD45-Positive Immune Cells Isolated from Treated Tumor

Briefly, C57BL/6J mice (11 male and 11 female) received SC injections of 105 B16-F10 cells and 8 days later were randomly placed into three groups. Transcriptome sequencing groups are as follows:

Group I: received only an IV injection of 1% HSA vehicle (n=6 mice; 3 female and 3 male) via retroorbital sinus injection under anesthesia

Group II: received an IV injection of 11.1 kBq of [225Ac]αMSH-PEG-Cy5-C′ dot (n=10 mice; 5 female and 5 male)

Group III: received an IV injection of unlabeled αMSH-PEG-Cy5-C′ dot (n=6 mice; 3 female and 3 male)

Based on the immune cell imaging analyses (as presented above), all mice were euthanized 96 hours post treatment and tumor harvested. The tumor was dissociated into single-cell suspensions using the Tumor Cell Isolation Kit (Miltenyi Biotec, catalog #130-096-730) for 45 minutes at 37° C. with shaking. The single-cell suspensions were individually passed through a 70 μm strainer to isolate single cells. The cells were then pelleted and resuspended in RPMI media. CD45 Microbeads (Miltenyi Biotec, catalog #130-052-301) were added to separate CD45-positive (CD45+) cells from the suspension. The CD45+ cells isolated from tumor were counted and stored at −80° C. in Trizol.

RNA was extracted from cells with chloroform and isopropanol. Linear acrylamide was then added to the RNA extract. The RNA was precipitated with 75% ethanol. Samples were resuspended in RNase-free water, and quality controlled using an Agilent BioAnalyzer. Transcriptome sequencing used 500 ng of total RNA from each tumor which underwent polyA selection and TruSeq library preparation according to instructions provided by Illumina (TruSeq Stranded mRNA LT Kit, catalog #RS-122-2102), with 8 cycles of PCR. Samples were barcoded and run on a HiSeq 4000 or HiSeq 2500 in rapid mode in a 50bp/50bp paired end run, using the HiSeq 3000/4000 SBS Kit or HiSeq Rapid SBS Kit v2 (Illumina). An average of 46 million paired reads was generated per sample. Ribosomal reads were not detectable, and the percent of mRNA bases averaged 74%.

### Bioinformatics Pipeline

Output data (FASTQ files) were mapped to the mouse genome (Genome: UCSC MM10) using the rnaStar aligner that maps reads genomically and resolves reads across splice junctions. A 2-pass mapping method was employed in which reads are mapped twice. The first mapping pass uses a list of known annotated junctions from Ensemble. Novel junctions found in the first pass are then added to the known junctions and a second mapping pass is done (n.b., on the second pass the RemoveNoncanoncial flag is used).

After mapping the output, SAM files were post processed using the PICARD tools to add read groups (i.e., AddOrReplaceReadGroups) which sorts the files and converts them to the compressed BAM format. The expression count matrix was computed from the mapped reads using HTSeq and mouse gene model database (GTF:Mus_musculus.GRCm38.80). The raw count matrix generated using HTSeq was then processed using the R/Bioconductor package DESeq, which is used to both normalize the full dataset and analyze differential expression between sample groups. Heatmaps were generated using the heatmap.2 function from the gplots R package. For heatmaps of (a) the top 100 differentially expressed genes and (b) top 71 differentially expressed cytokines, a cut-off of FC=2 and FDR=0.05 were used. The data were plotted as the mean centered normalized 1og2 expression.

### Computational Biology

Transcriptome data obtained from the CD45+ cells isolated from tumor were used to infer mouse immune signatures, cytokine expression, and pathways. Three phenotype classes were considered for this analysis as listed below:

(a) a vehicle-treated control group (n=6)

(b) the [²²⁵Ac]αMSH-PEG-Cy5-C′ dot-treated group (n=9)

(c) an unlabeled αMSH-PEG-Cy5-C′ dot-treated control group (n=6)

The CIBERSORT deconvolution method and ImmuneCC signatures were used to calculate the relative immune cell fractions. CIBERSORT was run on the normalized counts matrix using mouse signature genes derived from the ImmuneCC signature. Some genes from the immune signature matrix were not present in the count matrix (i.e., they had zero counts across all samples in experiments) and were excluded from the analysis. Pathway enrichment analysis was performed using the DAVID functional annotation tool.

### Statistical Analyses

Graphs were constructed using Prism (Graphpad Software Inc.) and Kaplan-Meier analysis applied for survival curve analysis. Statistical comparisons between the experimental groups were performed by Student's t-test (unpaired, two-tailed), or log-rank/Mantel-Cox test depending on the analysis. Multiple t-test analysis of the immune cell fractions used the method of Benjamin, Krieger and Yekutieli to examine P value distributions and estimate the fraction of true null hypotheses using a false discovery rate of 1%.

### Results

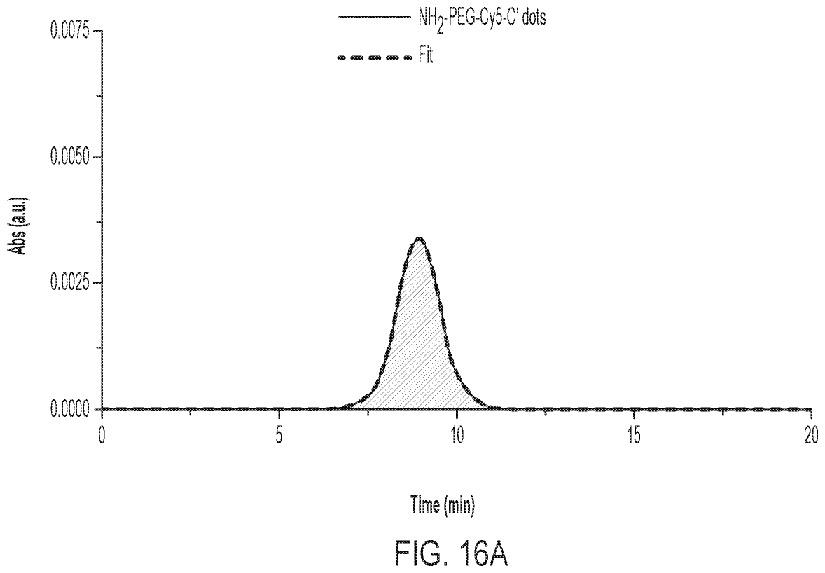

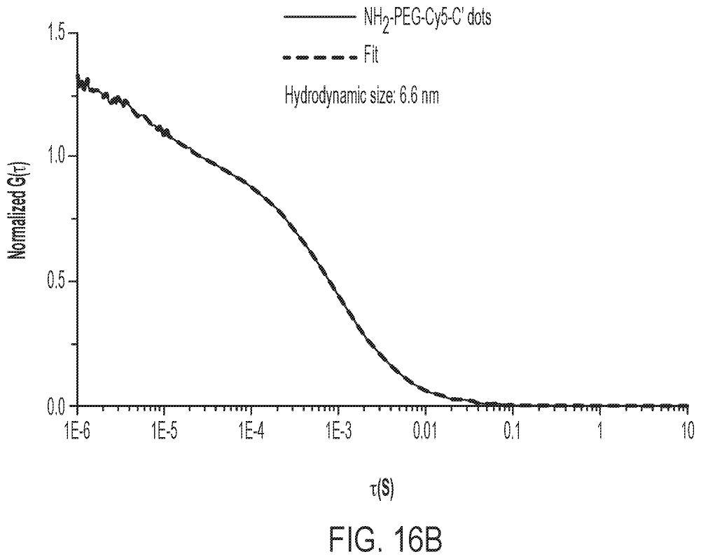

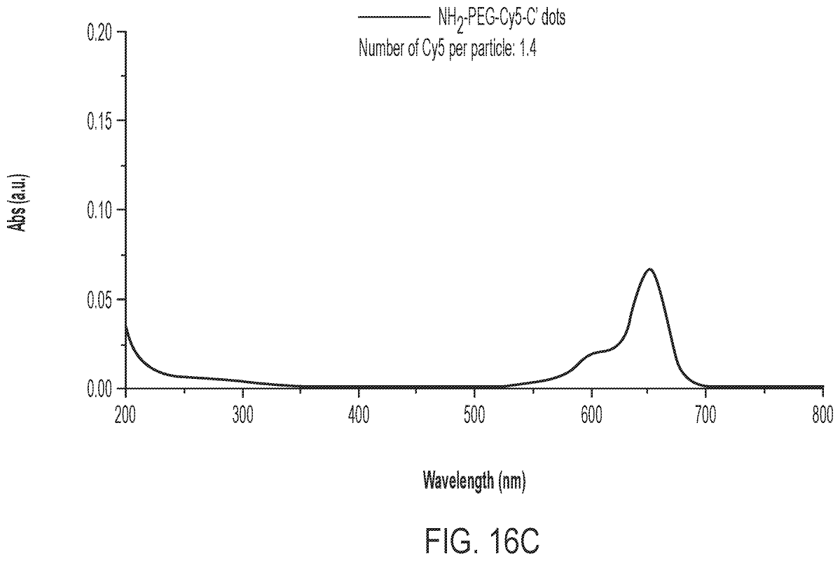

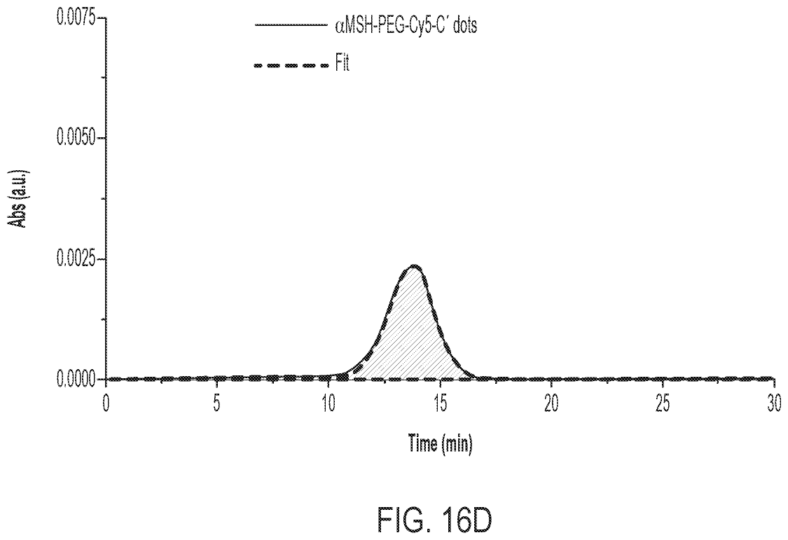

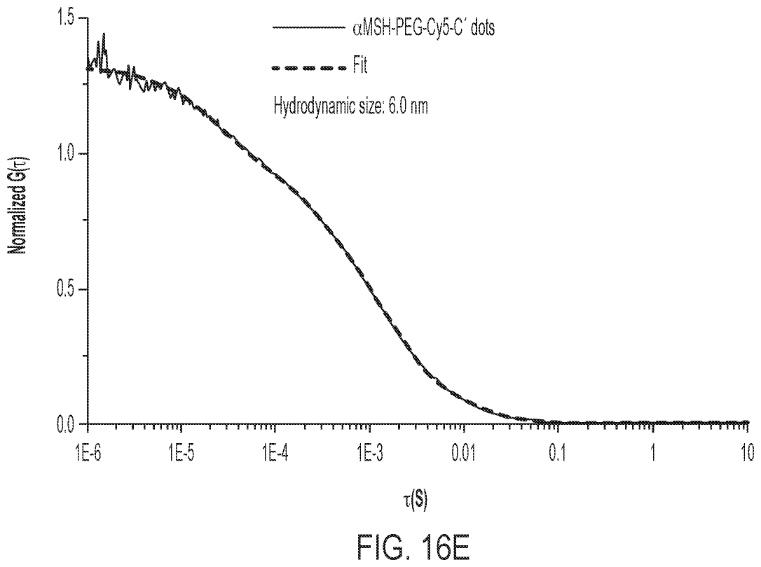

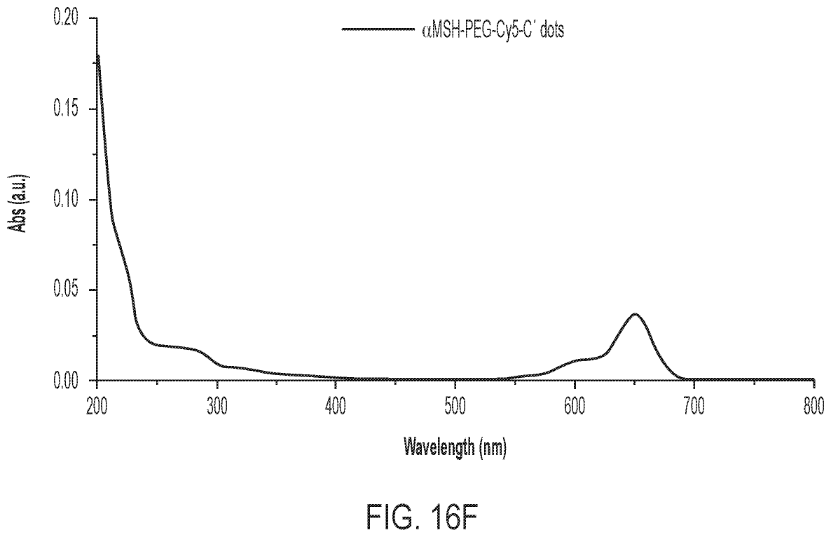

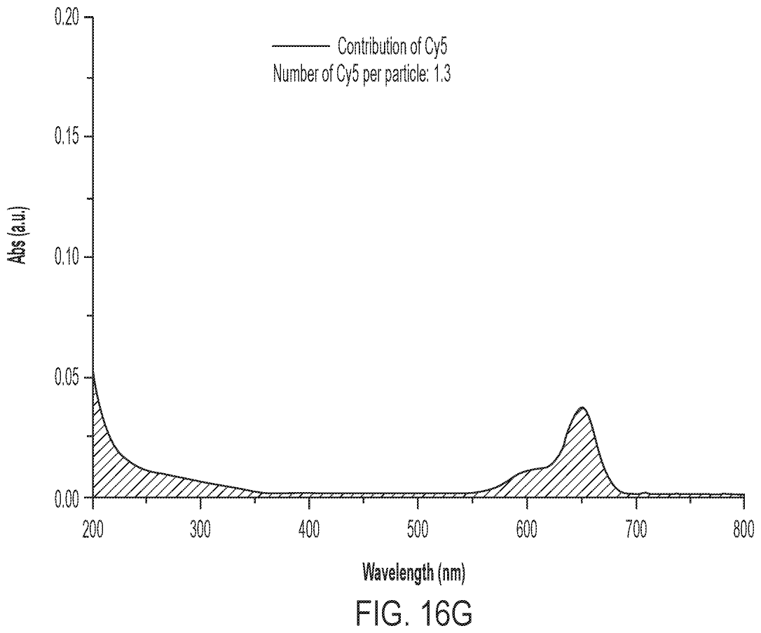

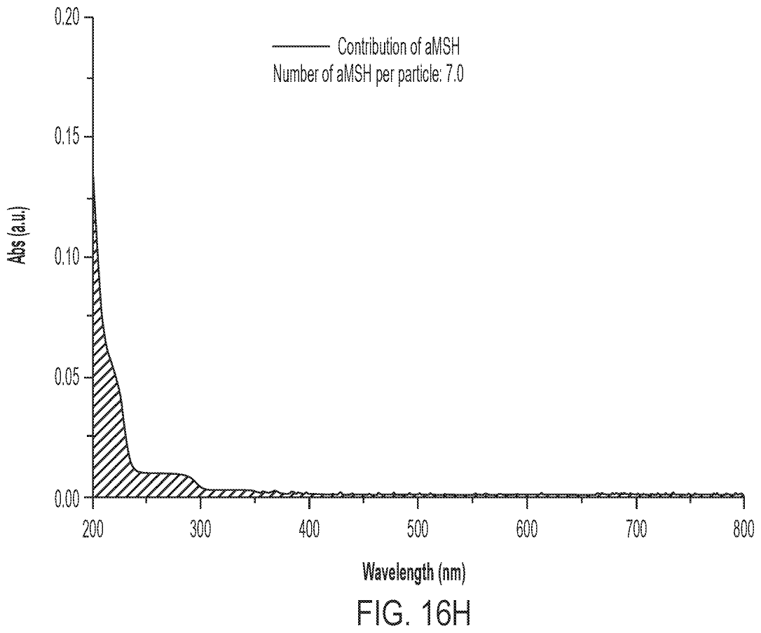

A sol-gel silica synthetic approach using water as solvent and polyethylene glycol (PEG) layer as shell yielded spherical, water-soluble ultrasmall fluorescent core-shell silica nanoparticles (C′ dots) with a narrow size distribution. These fluorescent core-shell silica nanoparticles have a 6.0 nm diameter and comprised, on average, 1.3 Cy5 dyes and 7.0 αMSH peptides (FIG. 16A-H).

FIGS. 16A-H show characterization data for NH₂-PEG-Cy5-C′ dots and αMSH-PEG-Cy5-C′ dots, respectively. FIG. 16A and FIG. 16D are GPC elugrams of NH₂-PEG-Cy5-C′ dots and αMSH-PEG-Cy5-C′ dots, respectively, with the corresponding curve fits. The absolute GPC elution times are not comparable since these chromatograms were taken on different days using different columns.

FIGS. 16B and 16E are FCS curves with fits of NH₂-PEG-Cy5-C′ dots and αMSH-PEG-Cy5-C′ dots, respectively. These data show that the hydrodynamic size of NH₂-PEG-Cy5-C′ dots is about 6.6 nm, and the hydrodynamic size of αMSH-PEG-Cy5-C′ dots is about 6.6 nm.

FIGS. 16C and 16F show UV-Vis absorbance of NH₂-PEG-Cy5-C′ dots and αMSH-PEG-Cy5-C′ dots, respectively. UV-Vis spectrum deconvolution of αMSH-PEG-Cy5-C′ dots in FIG. 16F shows the contributions of the absorbance spectra of Cy5 dye (as seen in FIG. 16G) and αMSH peptide (as seen in FIG. 16H), respectively, to the overall spectrum.

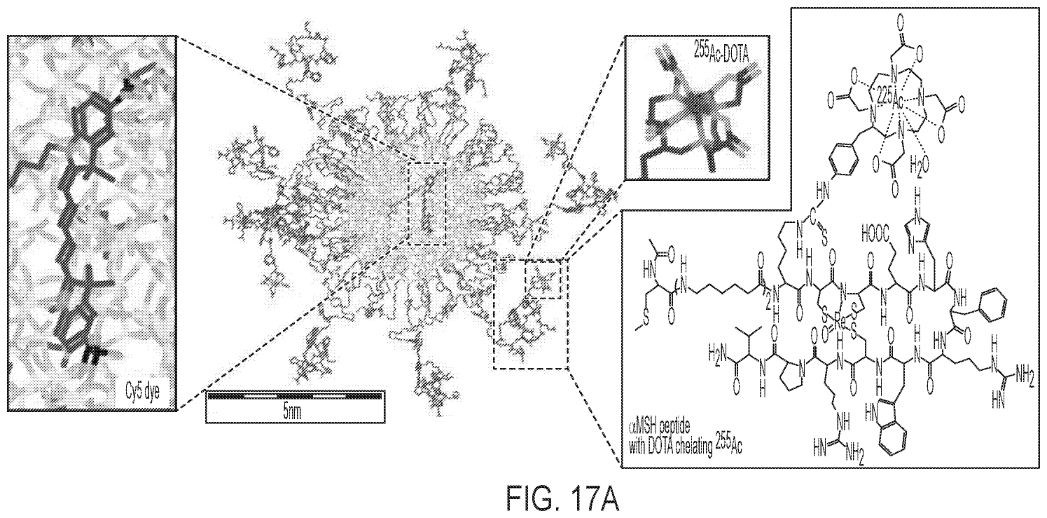

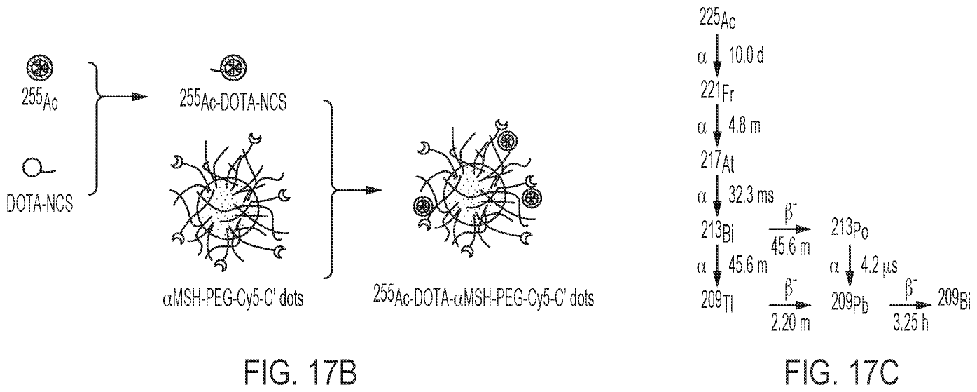

FIG. 17A shows a representation of the molecular structure of [²⁵⁵Ac]αMSH-PEG-Cy5-C′ dots. An illustration of the particle composition with silicon, oxygen, carbon, nitrogen, sulfur, and actinium atoms color are coded as purple, red, gray, blue, yellow and orange, respectively. Hydrogen atoms are not displayed. FIG. 17B shows an illustration of the radiosynthesis of [²⁵⁵Ac]αMSH-PEG-Cy5-C′ dots as discussed herein. Using a two-step radiochemical process as discussed herein, (i) ²⁵⁵Ac radioisotope is first chelated by DOTA-NCS and subsequently (ii) conjugated to primary amine functional groups on the αMSH peptide. FIG. 17C shows an illustrative representation of Actinium-225 decay. Each ²²⁵Ac radionuclide decay yields an alpha particle as well as several alpha emitting daughters.

In certain embodiments, radiochemical labeling methods to produce [²⁵⁵Ac]αMSH-PEG-Cy5-C′ dots (e.g., as seen in FIG. 17A) and [²⁵⁵Ac]NH2-PEG-Cy5-C′ dots are based on a two-step labeling approach and are illustrated in FIG. 17B. This radiochemical methodology has been designed to radiolabel temperature-sensitive proteins. In step one, ²²⁵Ac nitrate (0.023±0.014 GBq (mean±standard deviation); n=8) is bound by the bifunctional DOTA-Bz-SCN (0.66±0.27 mg; n=8) chelate. The bifunctional DOTA-Bz-SCN controls the pharmacokinetic fate of radionuclide dispersal in vivo and avoids nonspecific binding of radionuclide onto the C′ dots. This first reaction proceeds to 100% completion (n=8) under these conditions.

In step two, [²²⁵Ac]DOTA-Bz-SCN was reacted with the dLys epsilon amino group on the αMSH analogue (Ac-Cys-(aminohexanoic acid)2-dLys-Re[Cys-Cys-Glu-His-dPhe-Arg-Trp-Cys]-Arg-Pro-Val-NH2) via the reactive isothiocyanate moiety. The resulting [²²⁵Ac]DOTA-Bz-SCN product was then added directly to αMSH-PEG-Cy5-C′ dot (2.8 ±1.6 nmoles; n=6) and reacted for 42±16 min. (n=6). Purified [²²⁵Ac]αMSH-PEG-Cy5-C′ dots were isolated using size exclusion chromatography (SEC) and assayed for radiochemical purity at secular equilibrium (97.8 ±2.0%; n=6). The radiochemical yield of the second step was 2.9±1.8% (n=6). The specific activity was 236,115±106,722 GBq/mol; the activity concentration was 1.53±2.06 GBq/L; and the αMSH-PEG-Cy5-C′ dot concentration was 4.80 ±5.77 μmol/L, (all n=6).

The [²²⁵Ac]NH2-PEG-Cy5-C′ dot has a hydrodynamic diameter of 6.6 nm and 1.4 Cy5 dyes per particle (see, e.g. FIGS. 16B-C). The [²²⁵Ac]NH2-PEG-Cy5-C′ dots used as a nonspecific control (3.0±2.1 nmoles of C′ dot, n=2) were reacted for 35±7 min. and were determined to be 98.9±0.21% radiochemically pure. The radiochemical yield of the second step was 7.5±6.0% (n=2). The C′ dot control had specific activity of 250,778±40,698 GBq/mol; activity concentration 0.37±0.29 GBq/L; and NH₂-PEG-Cy5-C′ dot concentration of 1.42±0.94 μmol/L, (n=2).

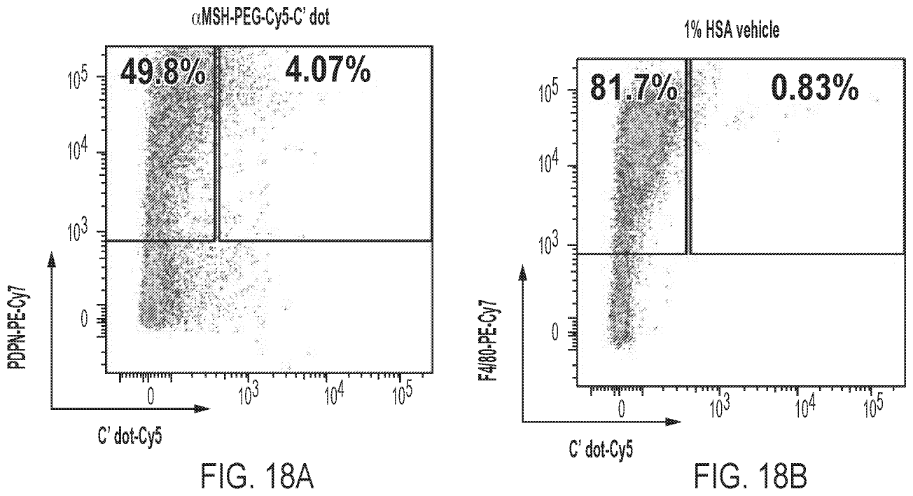

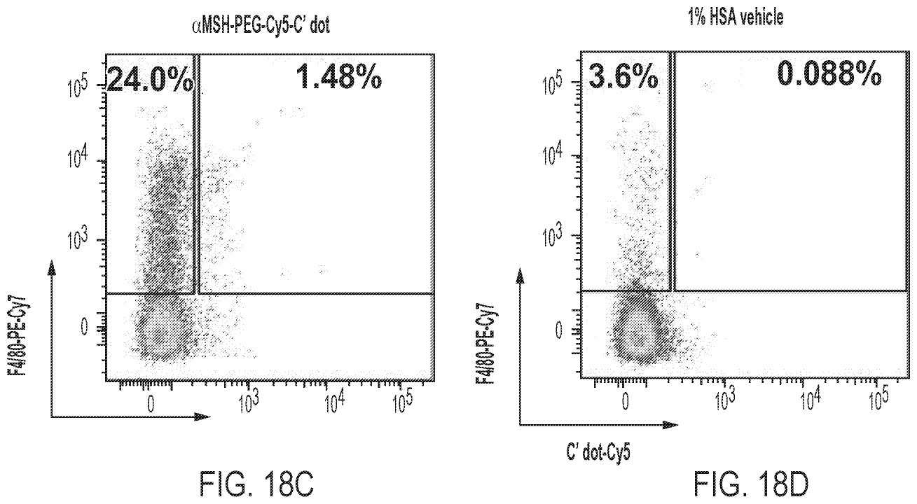

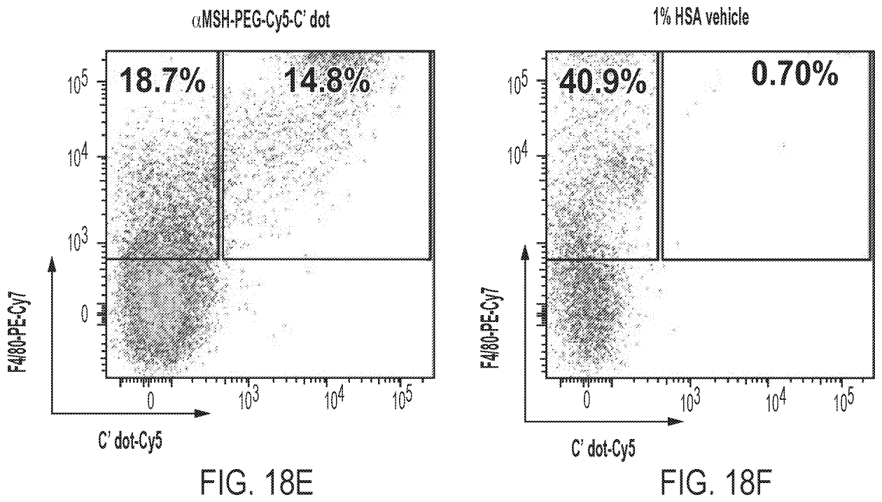

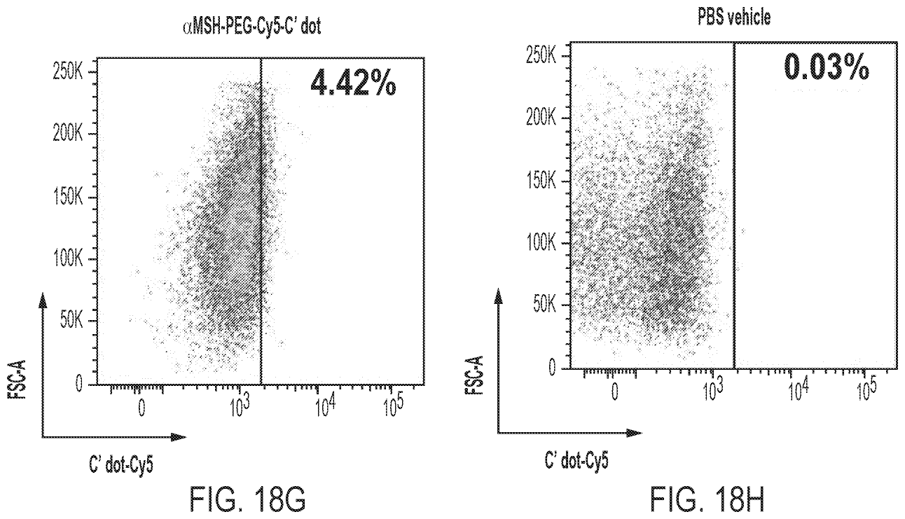

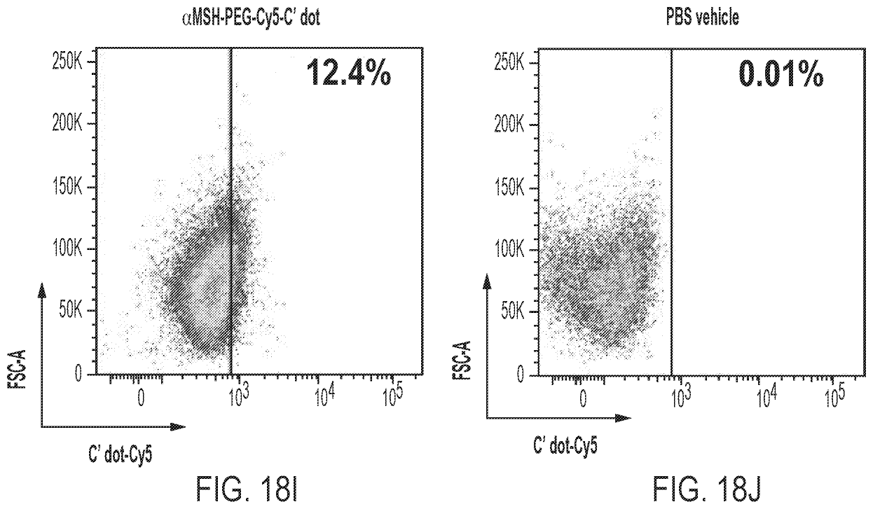

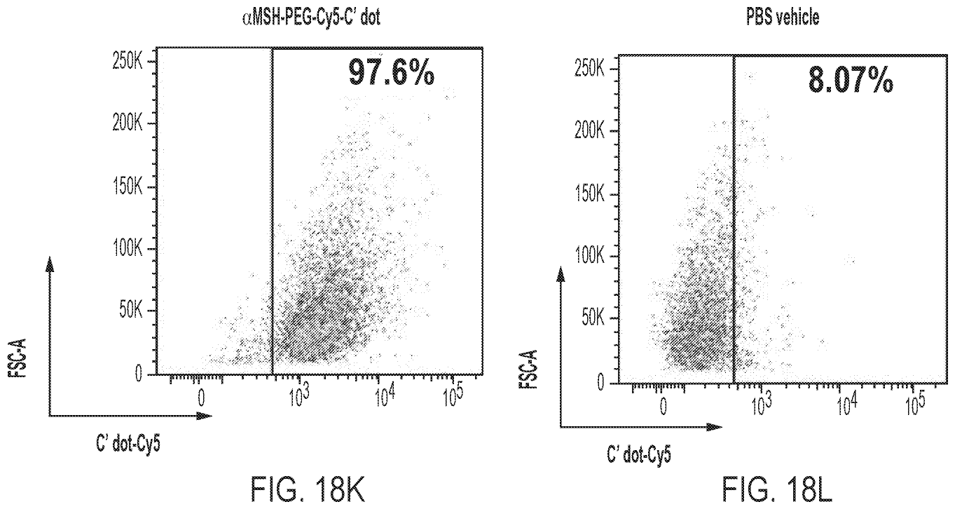

Flow cytometry investigations of tumor cells and macrophages confirmed that both cell types internalized αMSH-PEG-Cy5-C′ dots in vivo and in vitro. Intravenously administered αMSH-PEG-Cy5-C′ dots in mice with B16-F10 melanoma showed accumulation of the silica nanoparticles in both PDPN+ melanoma cells (4.07%) (FIG. 18A) and F4/80+ macrophages (1.48%) (FIG. 18C) as compared to the 1%HSA vehicle (FIGS. 18B and 18D). αMSH-PEG-Cy5-C′ dots administered intraperioneally to naive mice were also found to localize in the IP tissue macrophage population (14.8%) (FIG. 18E) versus vehicle (0.70%) (FIG. 18F). FACS analyses for both these experiments in vivo used the 1% HSA vehicle (containing no αMSH-PEG-Cy5-C′ dots) as a control (see FIGS. 18B, 18D, and 18F).

Tissue culture experiments also established αMSH-PEG-Cy5-C′ dots were internalized by B16-F10 cells (4.42%) (FIG. 18G), wild type THP-1 cells (12.4%)(FIG. 18I), and PMA-differentiated THP-1 cells (97.6%) (FIG. 18K) at 48 hours. FACS analyses of experiments in vitro used the PBS vehicle (no αMSH-PEG-Cy5-C′ dots) as a control (see FIG. 18H, 18J, 18L).

Experiments in vitro also indicated slower uptake kinetics where fewer C′ dots were internalized at 1 day than 2 days. C′ dot internalization plateaued at 2 days with only minimal additional accumulation at 3 and 4 day time points in the B16-F10, THP-1 and PMA-differentiated THP-1 cells.

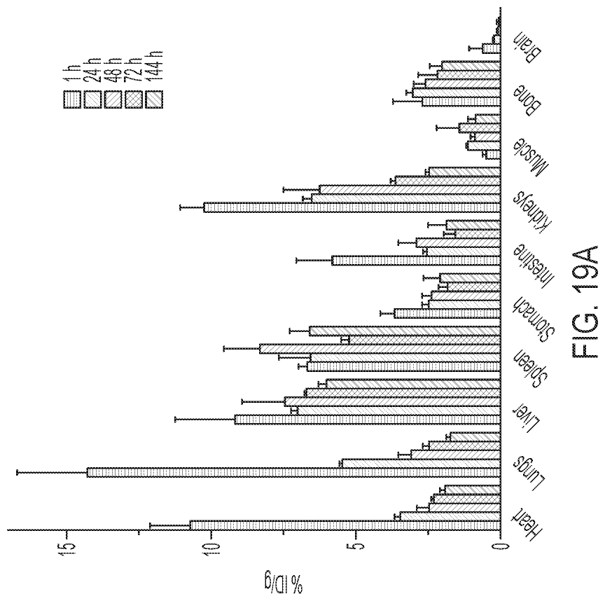

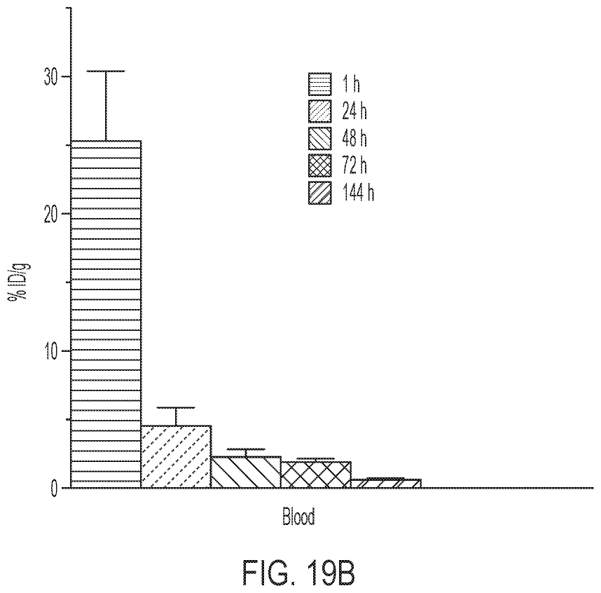

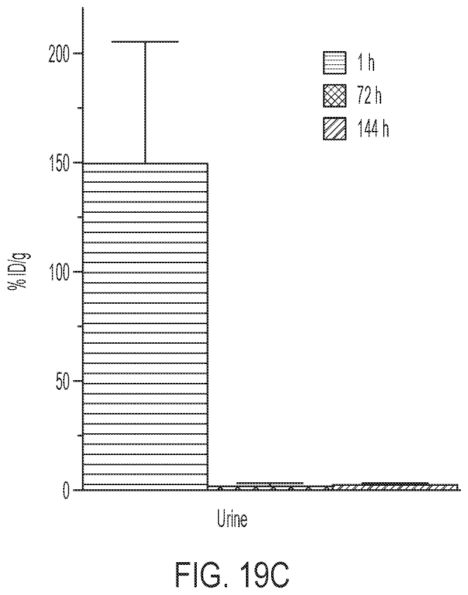

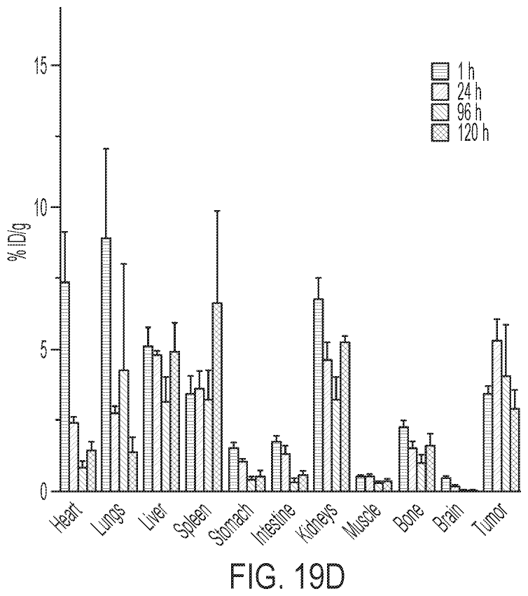

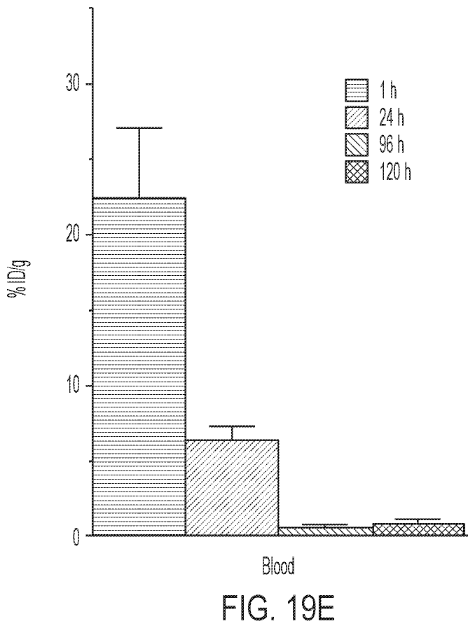

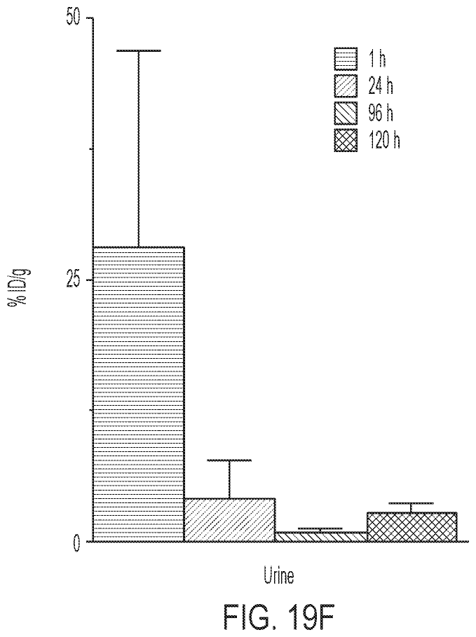

Pharmacokinetic data describing tissue biodistribution, blood clearance and renal elimination of [²²⁵Ac]αMSH-PEG-Cy5-C′ dot in healthy naive animals is shown in FIGS. 19A-C. Data in the figures are reported as the mean±standard error of the mean (SEM). Measurements were taken at 1, 24, 96 and 120 hours post injection.

²²⁵Ac activity in the blood compartment (FIG. 19B) dominates the pharmacokinetic profile at early time points (25.37±8.87%ID/g at 1 hour post injection; n=3) and is accompanied by rapid renal clearance (see FIG. 19C) (149.9±96.1%ID/g at 1 hour; n=3) of the ultrasmall silica particles. Blood activity decreases during the first day in vivo to 4.59±2.24%ID/g (n=3) at 24 hours post injection and further urinary excretion is minimal (<2.5%ID/g). The sum of the mean %ID that accumulated in all harvested tissues from each animal (n=10) is 11.12±1.58 and there is on average only 1.11±0.12%ID per tissue. Liver, spleen and kidney (see, e.g., FIG. 19A) have the greatest accumulation of nanoparticles at 7.02±0.35%ID/g, 6.58±1.86%ID/g and 6.52±0.54%ID/g, respectively.

Parallel pharmacokinetic analyses of [²²⁵Ac]αMSH-PEG-Cy5-C′ dot tissue biodistribution (FIG. 19D), blood clearance (FIG. 19E) and renal elimination (FIG. 19F) in syngeneic melanoma engrafted mice is shown in FIGS. 19D-F. Again, the blood compartment activity dominates the pharmacokinetic profile at early time points (22.47±10.39%ID/g at 1 hour post injection; n=5) and is accompanied by the rapid renal clearance (28.07±42.15%ID/g at 1 hour; n=5) of untargeted C′ dots. Blood activity decreases to 6.37±2.19%ID/g; n=5) at 24 hours post injection and further urinary excretion of the C′ dots is low (<4%ID/g). The intravenously administered activity exhibits biphasic elimination kinetics with a phase 1 effective half-life of 0.46 days and phase 2 half-life of 8.1 days. Tumor (see, e.g., FIG. 19D) accumulates 5.30±1.71%ID/g (n=5) of the injected activity at 1 day and the retention has an effective half-life of 115.5 hours. The sum of the mean %ID that accumulated in all harvested tissues from each mouse (n=10), not including tumor, is 7.79 ±1.25 and there is on average only 0.78±1.25%ID per tissue. Liver has the greatest accumulation of nanoparticle (4.79±0.36%ID/g), while spleen and kidney have 3.61±1.38 and 4.62±1.38%ID/g, respectively.

The [²²⁵Ac]αMSH-PEG-Cy5-C′ dot absorbed dose to tumor is estimated to be 2,412 cGy/MBq. The normal-organ absorbed doses (see Table 1 below) ranged from only a few rads to a few tens of rads for the administered activity of 11.1 kBq of [²²⁵Ac]αMSH-PEG-Cy5-C′ dots.

TABLE 1
[²²⁵Ac]αMSH-PEG-Cy5-C′ dot Absorbed Doses
in Mice and in the 70 kg Reference Man

Absorbed Dose (cGy/MBq)

| Tissue | Mouse | Reference Man |
|---|---|---|
| Brain | 45 | 0.773 |
| Large Intestine | 472 | 2.23 |
| Stomach Wall | 1.55 | |
| Heart Wall | 877 | 1.80 |
| Kidneys | 4792 | 11.4 |
| Liver | 1275 | 53.22 |
| Lungs | 1574 | 2.68 |
| Muscle | 240 | 0.757 |
| Red Marrow | 1.25 | |
| Bone | 1290 | 60.0 |
| Spleen | 1275 | 4.41 |
| Total Body | 2.15 | |
| Tumor | 2412 | |

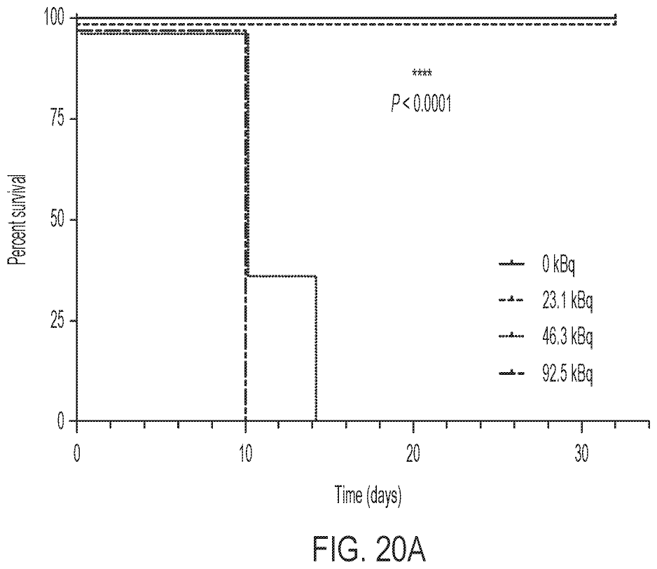

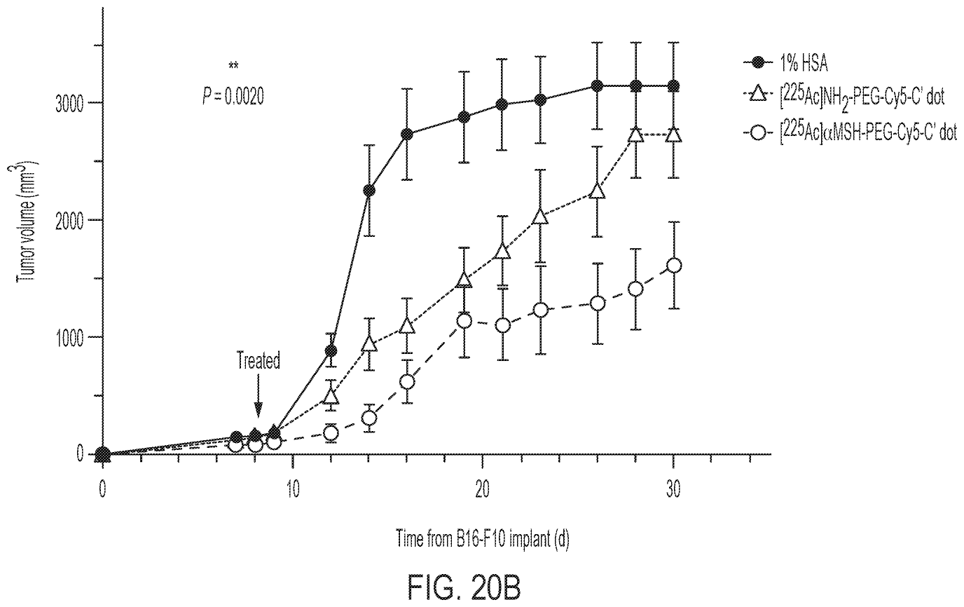

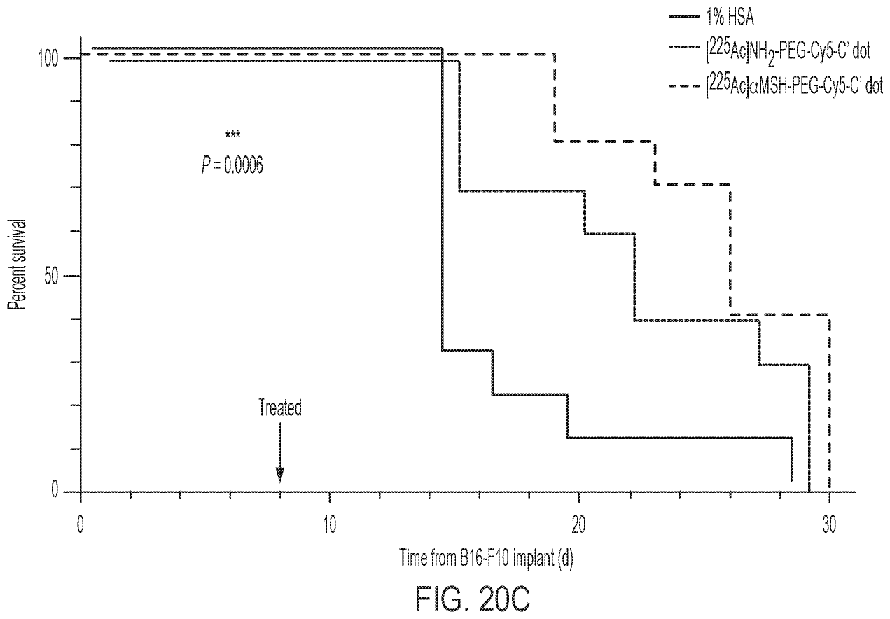

This also correlates with the observation that there was no pronounced normal-tissue toxicity in the pharmacokinetic or pharmacodynamic studies. Additional data for dose estimates to the normal organs in mice and to normal organs in the 70-kg Reference Man are presented in Table 1 above. The mouse absorbed doses on a per-MBq basis are much higher than the Reference Man dose, reflecting the orders of magnitude difference in body mass between mouse and human. In a human, the organ absorbed doses are uniformly of the order of 1 cGy/MBq except for 11.4 cGy/MBq delivered to the kidneys. The maximum tolerated dose (MTD) of [²²⁵Ac]αMSH-PEG-Cy5-C′ dot was at least 23.1 kBq (0.63 μCi per mouse) and below 46.3 kBq (1.26 μCi per mouse) in healthy, naive mice (FIG. 20A). Median survival was undefined in the groups that received 0 or 23.1 kBq and 10 days in mice that received either 46.3 or 92.5 kBq. Human dosimetry predictions (i.e., 70-kg man) for a 37 MBq dose of [²²⁵Ac]αMSH-PEG-Cy5-C′ dot predicted that the absorbed dose to kidney, liver and lung is 4.2, 1.9, and 0.99 Gy, respectively. These doses are significantly below the dose limits of 23, 40, and 20 Gy for these organs, respectively.

FIGS. 20A-C shows a pharmacodynamic profile of [²²⁵Ac]αMSH-PEG-Cy5-C′ dots bioactivity in naïve and syngeneic B16-F10 tumor-bearing C57BL/6J mice. FIG. 20A shows a determination of the maximum tolerated dose of [²²⁵Ac]αMSH-PEG-Cy5-C′ dots in naïve C57BL/6J mice (n=5 per group) that received 0, 23.1, 46.3, or 92.5 kBq per mouse. The curves are nudged to separate overlaying data for better visualization. Alpha particle radiotherapeutic effects on B16-F10 (FIG. 20B) tumor volume and (FIG. 20C) survival in C57BL/6J mice following a single intravenous dose of 11.1 kBq and 55 μmoles of specific [²²⁵Ac]αMSH-PEG-Cy5-C′ dot ; 11.1 kBq and 55 μmoles of non-specific [²²⁵Ac]NH₂-PEG-Cy5-C′ dot, or the 1% HSA injection vehicle. All three group sizes are n=10. The curves are nudged to separate overlaying data. Data are mean±SEM in FIG. 20B.

Pharmacodynamic studies examined B16-F10 tumor control, host survival and associated effects on the TME immune cell content using an immunocompetent mouse model of melanoma following a single IV administration of 11.1 kBq (300 nCi) of [²²⁵Ac]αMSH-PEG-Cy5-C′ dots. Control experiments included the injection of vehicle as a growth control and a non-specific [²²⁵Ac]NH2-PEG-Cy5-C′ dot particle. This therapy study employed a radioactivity dose approximately 50% lower than MTD to mitigate non-specific effects. Tumor volumes were measured longitudinally and presented in FIG. 20B. Linear tumor growth becomes exponential at approximately 10 days post implantation in the vehicle-treated growth control group. Non-specific radiation effects arising from the non-targeting particle delay the rate of tumor growth compared to the growth control. Specific tumor growth control is observed with a decrease of >50% tumor volume when compared to the vehicle group on day 30. Separation in the tumor volume curves is observed between the specific and non-specific groups throughout the course of the study. Kaplan-Meier analysis reports median survival times of 14, 21, and 26 days for the vehicle, non-specific, and specific groups, respectively (FIG. 20C). A Log-rank (Mantel-Cox) test shows a statistically significant difference (P=0.0020) in the survival data for all three groups (FIG. 20C). Comparison of the specific group with the vehicle control is statistically significant (P=0.0006) and a Hazard Ratio of 9.986 (95% confidence interval is 2.671 to 37.33) using the Mantel-Haenszel test.

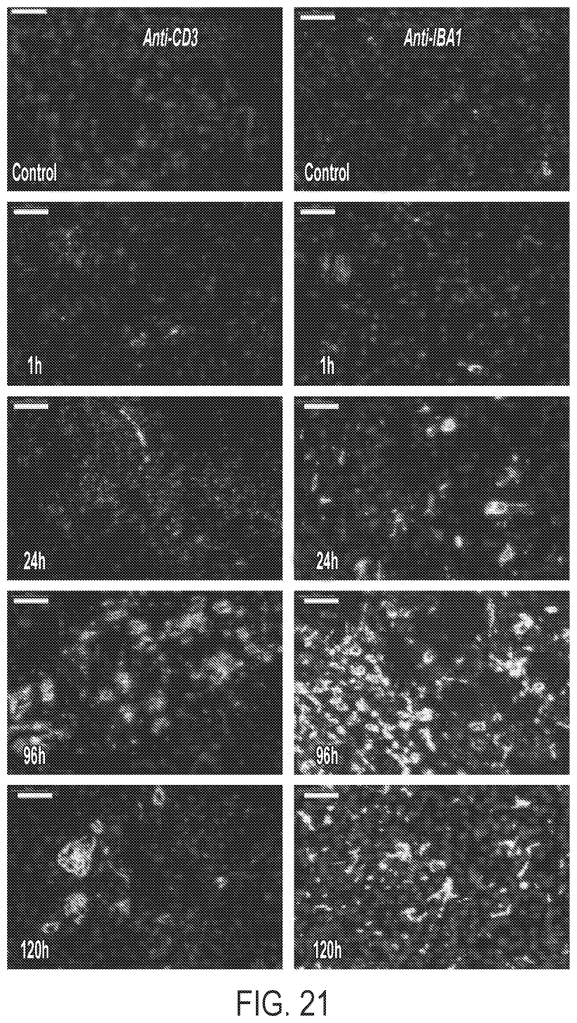

Immune cells populating the alpha-irradiated TME were characterized using IF staining of tumor harvested at different times after [²²⁵Ac]αMSH-PEG-Cy5-C′ dot-treatment. Distinct changes in immune phenotypes were observed as a function of time from treatment (FIG. 21). FIG. 21 shows representative images of immune cells in the B16-F10 tumor microenvironment. Tumor tissue was harvested at 1, 24, 96, and 120 hours post-treatment and stained with anti-CD3 (left column) or anti-IBA1 (right column) immunofluorescence markers to identify time-dependent changes in composition. These tumor samples were obtained from the animals in the pharmacodynamic therapy study as mentioned previously herein. Images of untreated tumor tissue are included as control. Immunofluorescence stains of T cells (green) and macrophages (green) are counterstained with DAPI (blue). Scale bars are 50 μm.

Anti-CD3 and anti-IBA1 staining shows time-dependent changes in T cells and macrophages in the TME. Image quantification demonstrates that T cell (CD3+) and macrophage (IBAl+) expression peaks 4 days following treatment.

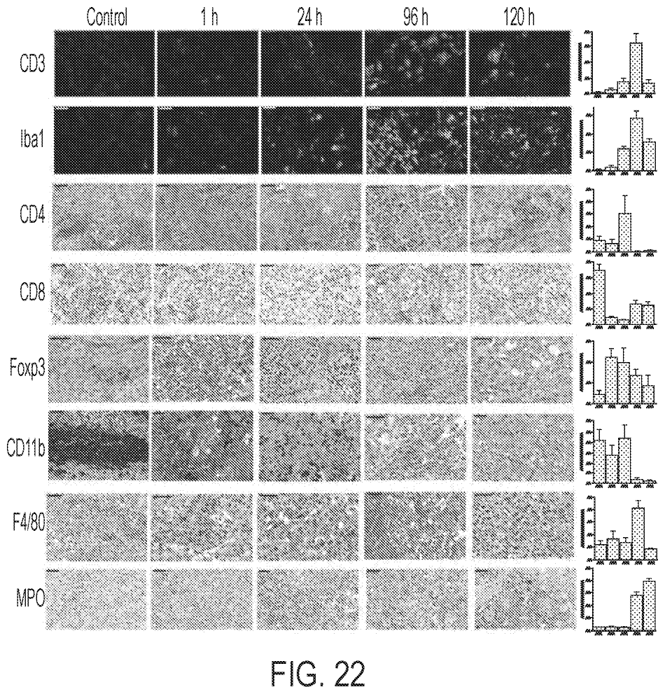

Furthermore, FIG. 22 also shows distinct changes in additional immune phenotypes. CD3, Iba 1, F4/80, CD4, CD8, Foxp3, CD11b, and myeloperoxidase (MPO) staining shows time-dependent increases and decreases of T cells, macrophages, and neutrophils in B16-F10 tumor-bearing mice. Image quantification shows that T cell (CD3+) and macrophage (lbal+and F4/80+) expression peaks 4 days following treatment. CD4 cell expression peaks at 1 day and then decreases; CD8 expression decreases after treatment relative to baseline tumor expression; Foxp3 expressing regulatory T cells increase as early as 1 hour post treatment and then decrease; CD11 b staining (leukocytes) is high at base line and persists for 1 day and then drops significantly by 4 days; neutrophils (MPO stained) have low expression during the first day and then dramatically increase at 4 days post-treatment and continue to increase.

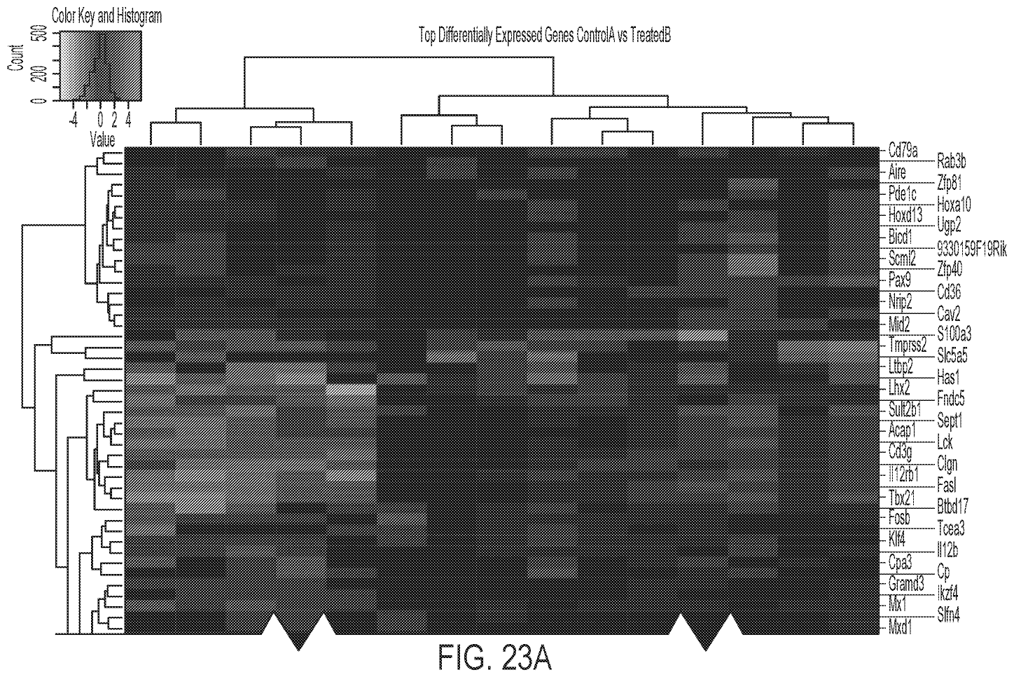

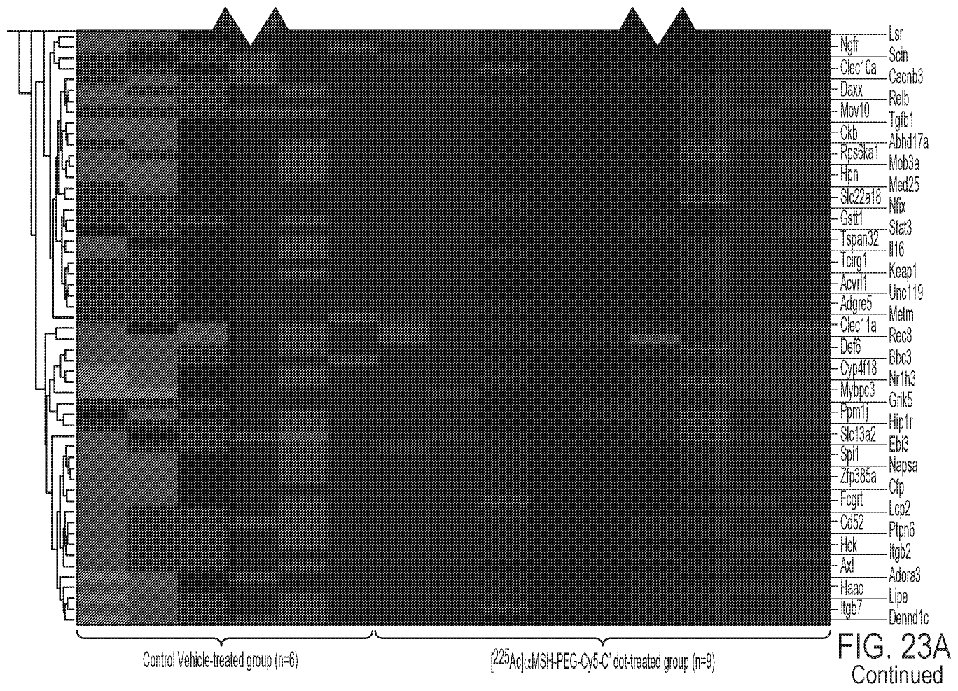

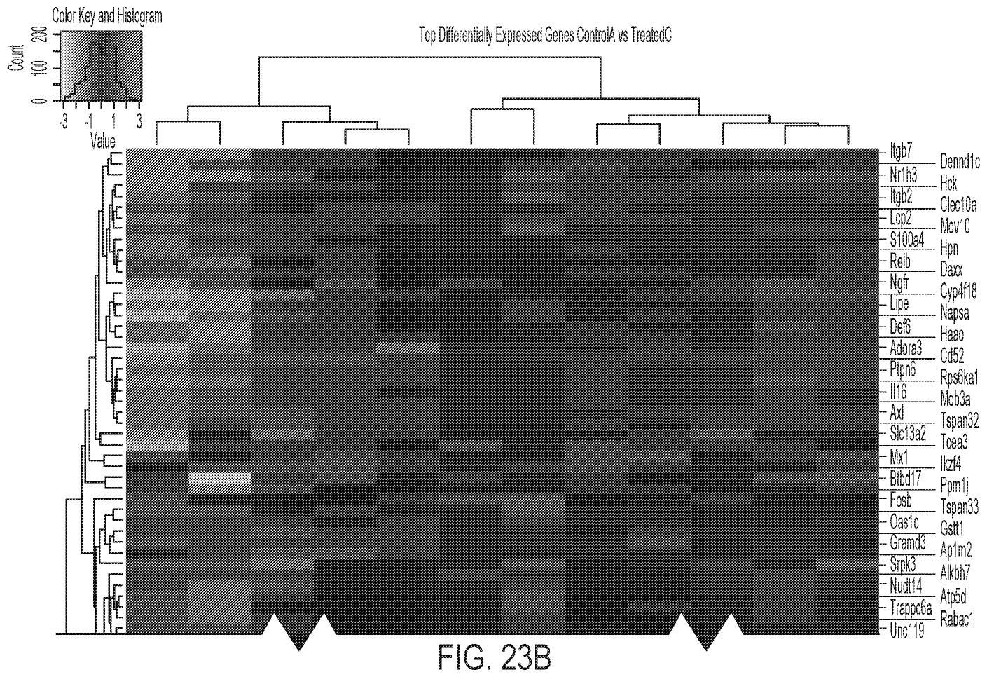

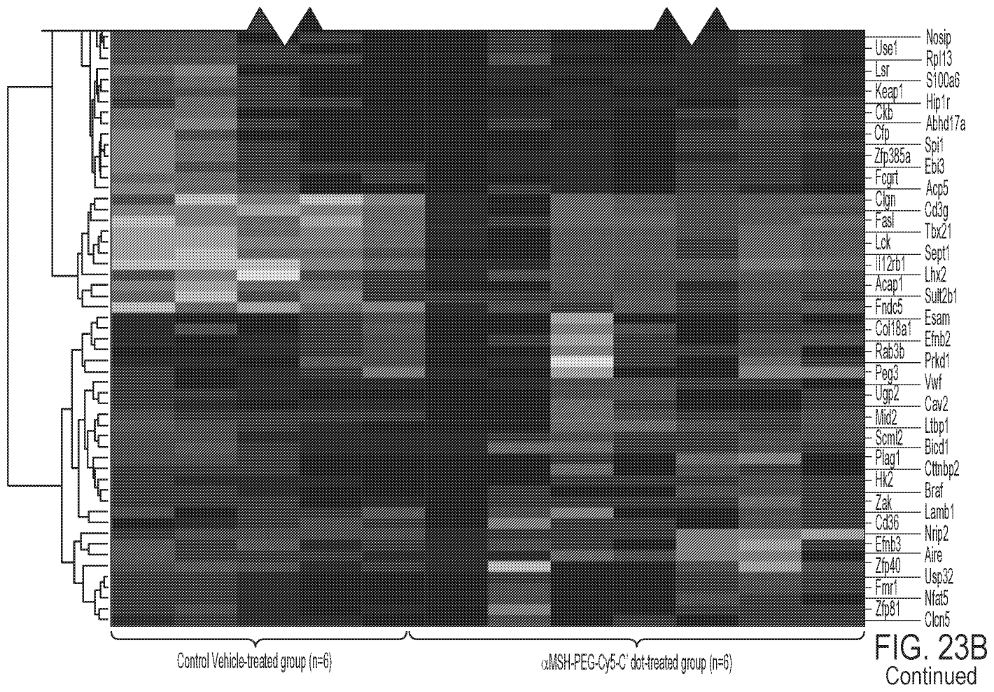

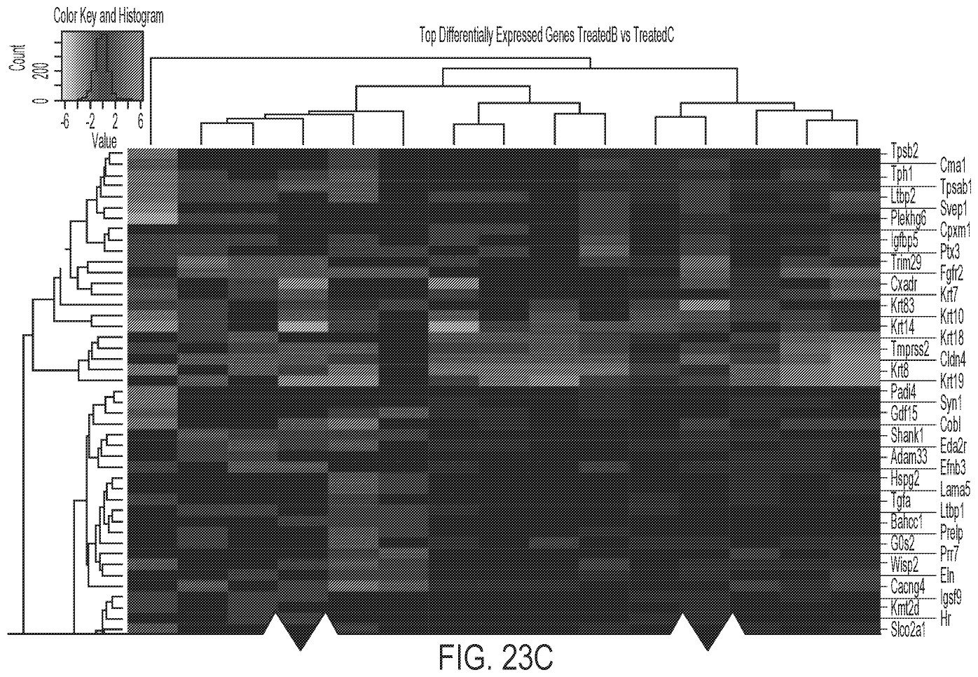

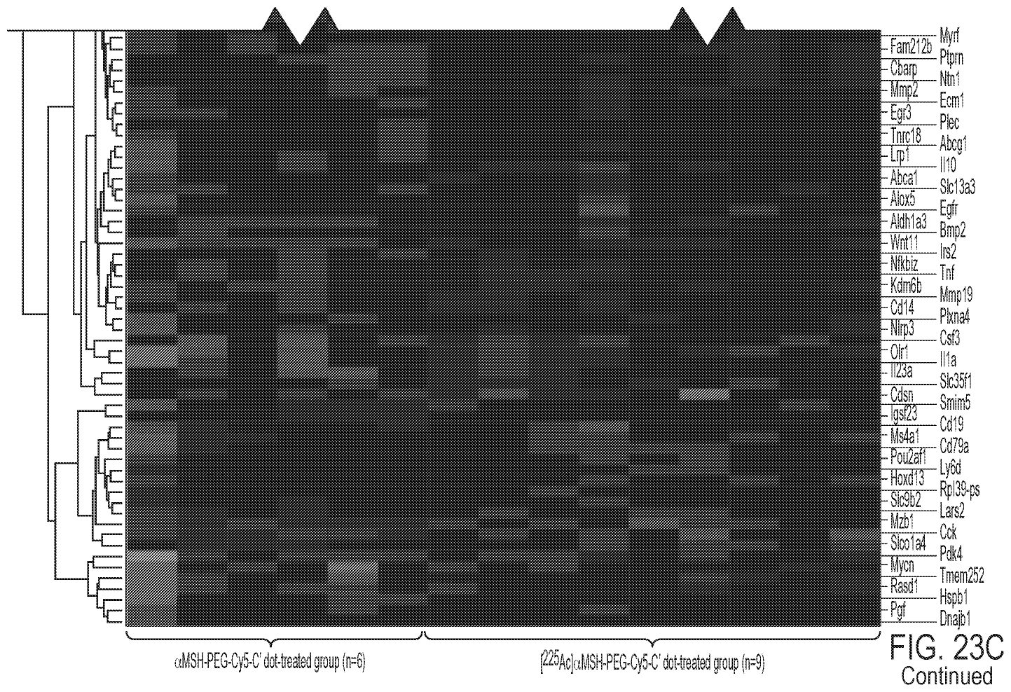

Transcriptome sequencing of all CD45-positive cells isolated from ‘hot’ [²²⁵Ac]αMSH-PEG-Cy5-C′ dot- and ‘cold’ αMSH-PEG-Cy5-C′ dot-treated tumors (and vehicle-treated controls) provided an extensive gene dataset to analyze the immune cell signatures in the TME at 96 hours post treatment. This time point was selected based on the results of the IF (immunofluorescent) experiments where maximal changes in T cell and macrophage numbers were observed in the TME (tumor microenvironment) versus untreated growth controls. Computational interrogation of differentially expressed genes in each group versus controls yielded heatmaps (FIGS. 23A-C) indicating patterns of up- and down-regulated genes.

FIG. 23A shows the top differentially expressed genes in an vehicle-treated control group (n=6) versus the [²²⁵Ac]αMSH-PEG-Cy5-C′ dot-treated group (n=9). FIG. 23B shows the top differentially expressed genes in an vehicle-treated control group (n=6) versus an unlabeled αMSH-PEG-Cy5-C′ dot-treated control group (n=6). FIG. 23C shows the top differentially expressed genes in the [²²⁵Ac]αMSH-PEG-Cy5-C′ dot-treated group (n=9) versus an unlabeled αMSH-PEG-Cy5-C′ dot-treated control group (n=6).

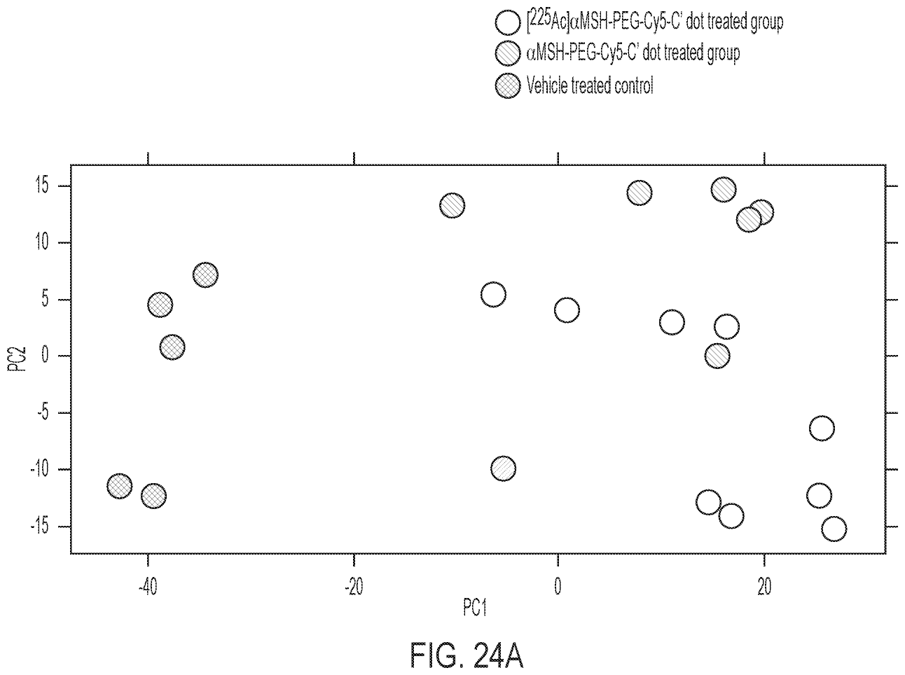

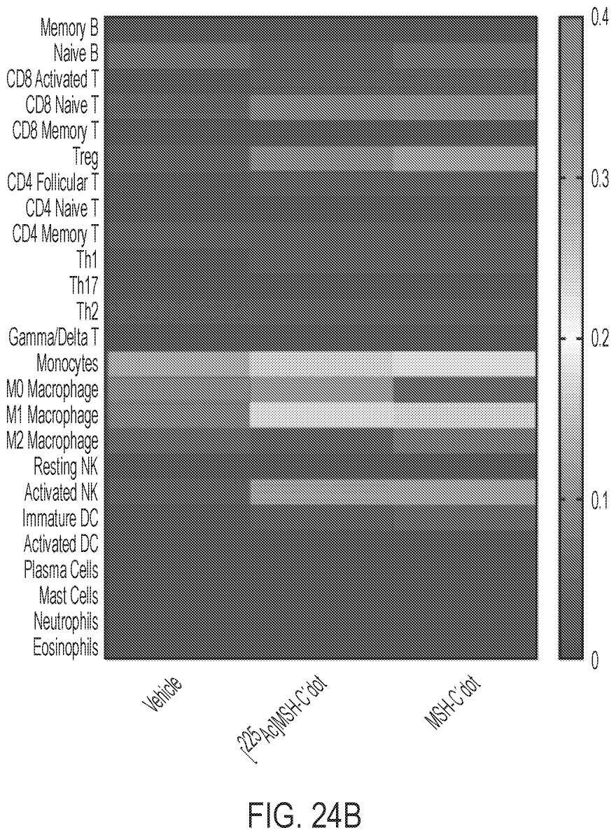

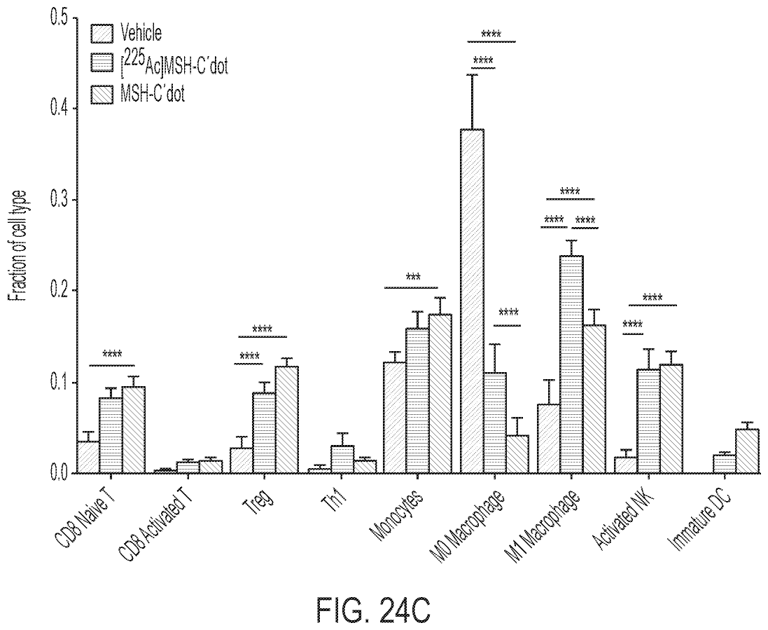

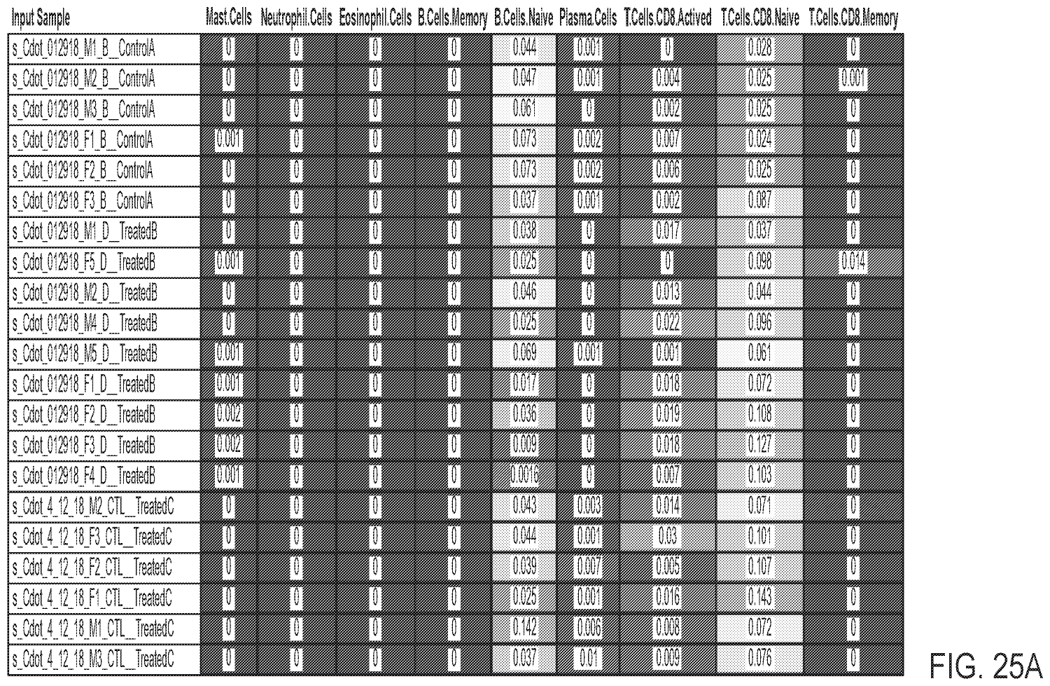

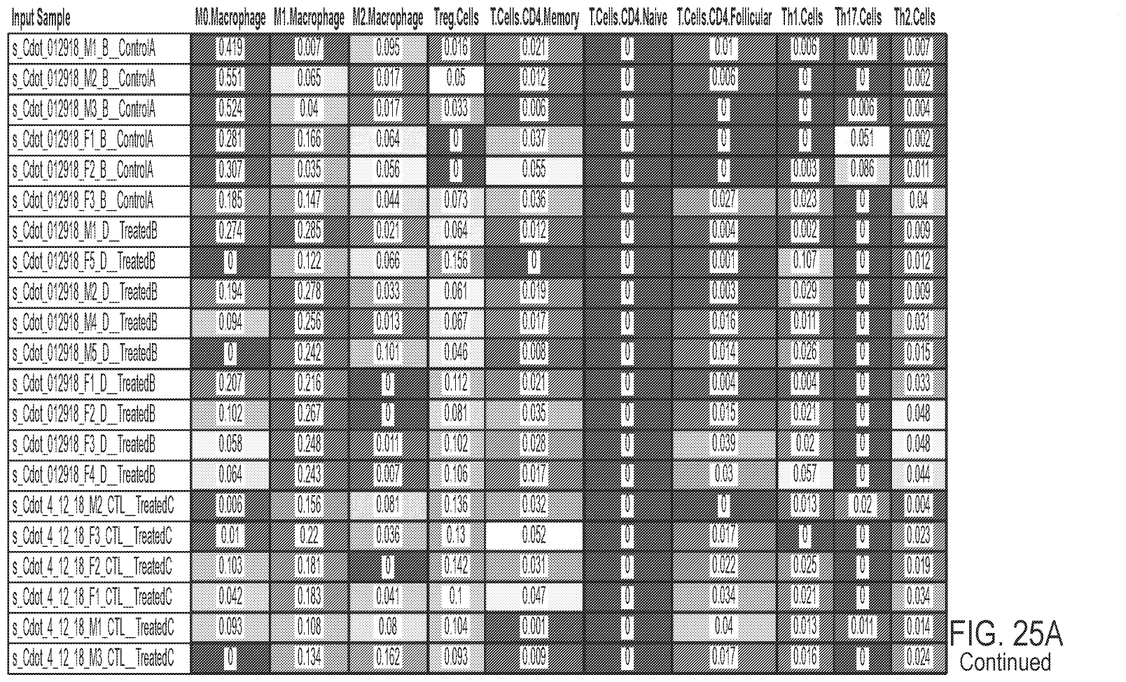

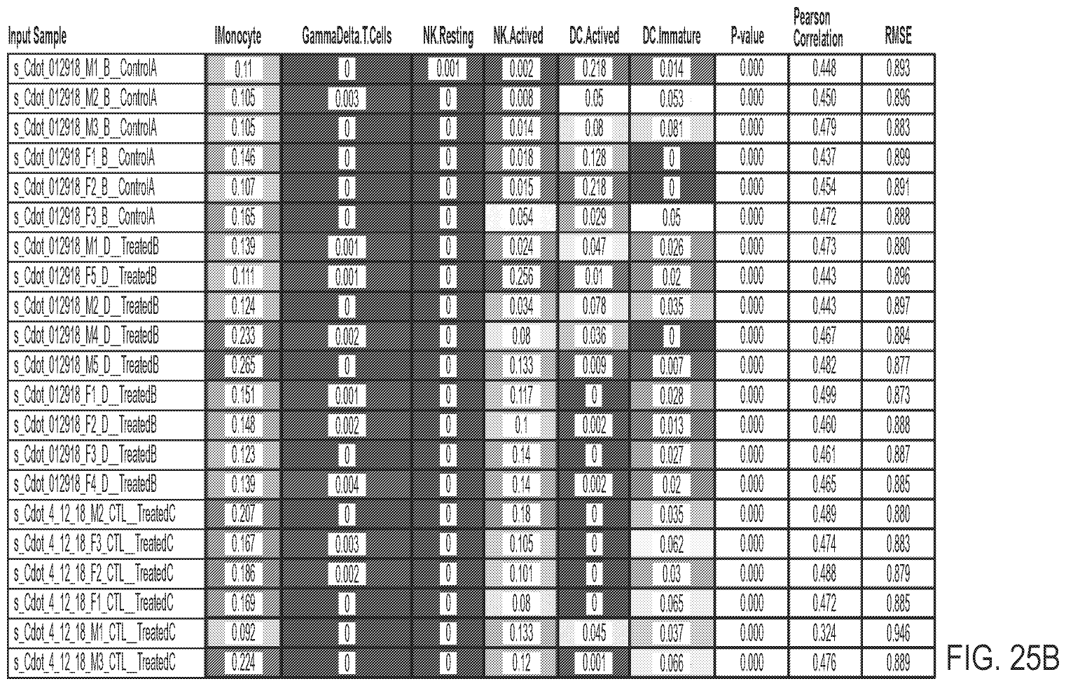

An unsupervised principal component analysis of these data (FIG. 24A) also demonstrated distinct treatment-based effects for both the radiolabeled and unlabeled targeted C′ dots relative to the vehicle-treated controls. FIG. 24A shows an unsupervised principal component analysis (PCA) showing the first two principal components of all samples using data obtained from RNA-seq of an untreated control group that received only vehicle, an [²²⁵Ac]αMSH-PEG-Cy5-C′ dot treated group, and an unlabeled αMSH-PEG-Cy5-C′ dot treated control group. These data were then evaluated to infer the relative fractions of immune cells in each tumor (FIG. 24B, FIG. 24C, and FIGS. 25A-B) using CIBERSORT and ImmuneCC algorithms. FIGS. 25A-B shows tabular RNA seq data obtained from the CIBERSORT and ImmuneCC analysis of 25 different murine immune cell signatures in 21 individual tumors.

Heat maps demonstrate important population shifts as a function of treatment and statistical analyses report significant increases in naïve CD8 T cells, T regulatory (Treg) cells, monocytes, MΦ and M1 macrophages and activated natural killer (NK) cells arising from either the ²²⁵Ac-labeled or unlabeled αMSH-PEG-Cy5-C′ dots compared to the vehicle-treated tumors (FIGS. 24B and 24C).

Innate immunity changes in the TME entail increases in the fraction of classically activated macrophages (M1) for both nanoparticle treatment groups (‘hot’ radiolabeled C′ dot is 0.2397±0.0486 (n=9) and ‘cold’ unlabeled C′ dot is 0.1636±0.0397 (n=6)) versus vehicle-treated controls (0.0766±0.0648 (n=6)). The TME monocyte content increased in the ‘hot’ (0.1592±0.05317) and ‘cold’ (0.1744±0.04579) treated groups relative to untreated controls (0.1231±0.02594). Infiltration of activated NK cells increases in ‘hot’ (0.1137±0.0686) and ‘cold’ C′ dot-treated tumors (0.1200±0.0346) versus vehicle-treated controls (0.0183±0.0184). The fraction of MO macrophages decreased significantly following treatment with either ‘hot’ (0.1105±0.09537) or ‘cold’ (0.0421±0.04568) targeted C′ dot treatment versus untreated controls (0.378±0.1448). Immature dendritic cells (DC) were not detected in vehicle-treated tumors but the fractions of these antigen-presenting cells increased in the ‘hot’ (0.0120±0.0111) and ‘cold’ (0.0493±0.0168) treated groups.

The adaptive immune response is also engaged and the fraction of activated CD8 T cells increased several-fold after both ‘hot’ (0.0129±0.0082) and ‘cold’ C′ dot treatment (0.0136±0.0091) relative to the vehicle-treated control groups (0.0036±0.0026). The fraction of naive CD8 T cells increased following ‘hot’ (0.08286±0.03066) and ‘cold’ C′ dot treatment (0.09497±0.02825) versus vehicle-treated controls (0.03557±0.02508). Similarly the fraction of Th1 cells increased in ‘hot’ (0.0307±0.0328) and ‘cold’ (0.0144±0.0085) treated animals versus untreated controls (0.0054±0.0090). Interestingly, the numbers of T regulatory cells also increased in the ‘hot’ (0.0884±0.03413) and ‘cold’ (0.1176±0.02088) treated animals versus the untreated controls (0.02852±0.02895).

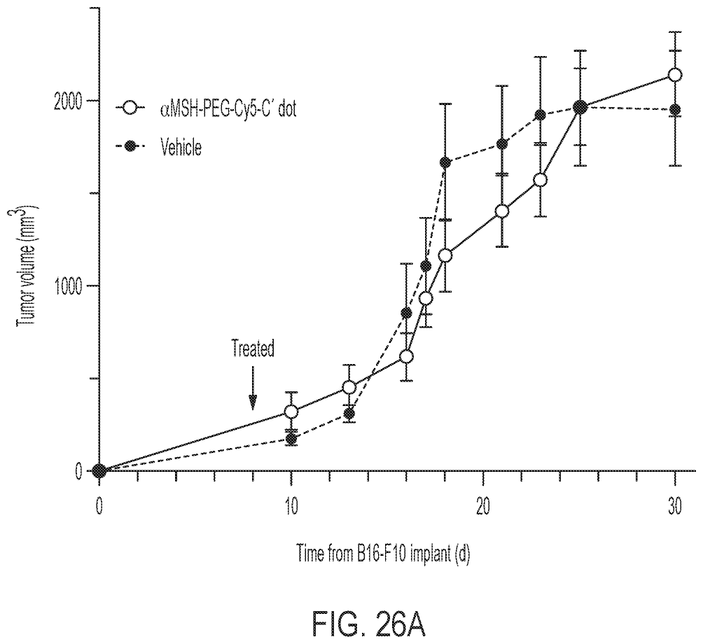

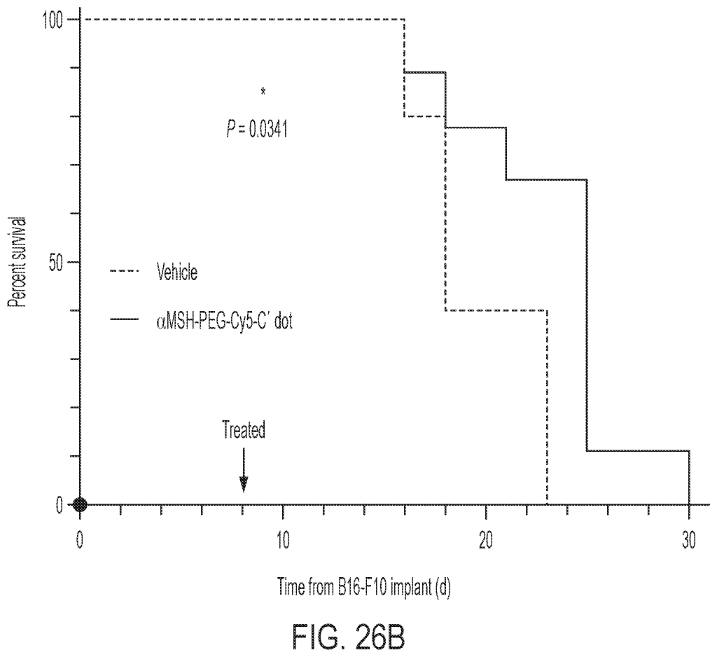

While the alpha particle radiotherapy study showed specific and potent tumor control derived from [²²⁵Ac]αMSH-PEG-Cy5-C′ dots (FIGS. 20B and 20C), an additional therapy study was included to investigate tumor control arising from a single administration of 55 μmole of unlabeled ‘cold’ αMSH-PEG-Cy5-C′ dots on day 8 versus vehicle-treated controls (FIGS. 26A-B). The unlabeled ‘cold’ C′ dots are not as cytotoxic nor as effective in controlling tumor growth as the ‘hot’ radiolabeled C′ dots (FIGS. 26A and 26B). FIG. 26A-B show (FIG. 26A) tumor volume measurements and (FIG. 26B) a survival plot of B16-F10 tumor-bearing C57BL/6J mice following a single intravenous dose of 55 pmoles of unlabeled (‘cold’) αMSH-PEG-Cy5-C′ dot (n=10) or the 1% HSA injection vehicle (n=5). Data are reported as the mean±SEM.

A slight delay in tumor growth is noted at 18 days versus the untreated controls. Kaplan-Meier analysis reports median survival times of 18 and 25 days for the vehicle and ‘cold’ C′ dots groups, respectively (FIG. 26B). A Log-rank (Mantel-Cox) test shows a statistically significant difference (P=0.0341) in the survival data for these two groups.

However, the transcriptome analysis shows that the ‘cold’ targeted particle does exert an effect on the immune cells populating the TME. Both ‘hot’ and ‘cold’ targeted C′ dots have comparable immune cell fractions compared to the vehicle-treated tumors. Without wishing to be bound to any particular theory, the C′ dot platform has a dominant role in TME local immunity. While it is evident that both labeled and unlabeled αMSH-PEG-Cy5-C′ dots prompt changes in CD8 T and Treg cells, monocytes, MΦ and M1 macrophages and activated NK cells, in certain embodiments the cytotoxic ²²⁵Ac component of the drug composition introduces a potently cytotoxic element and effects tumor control.

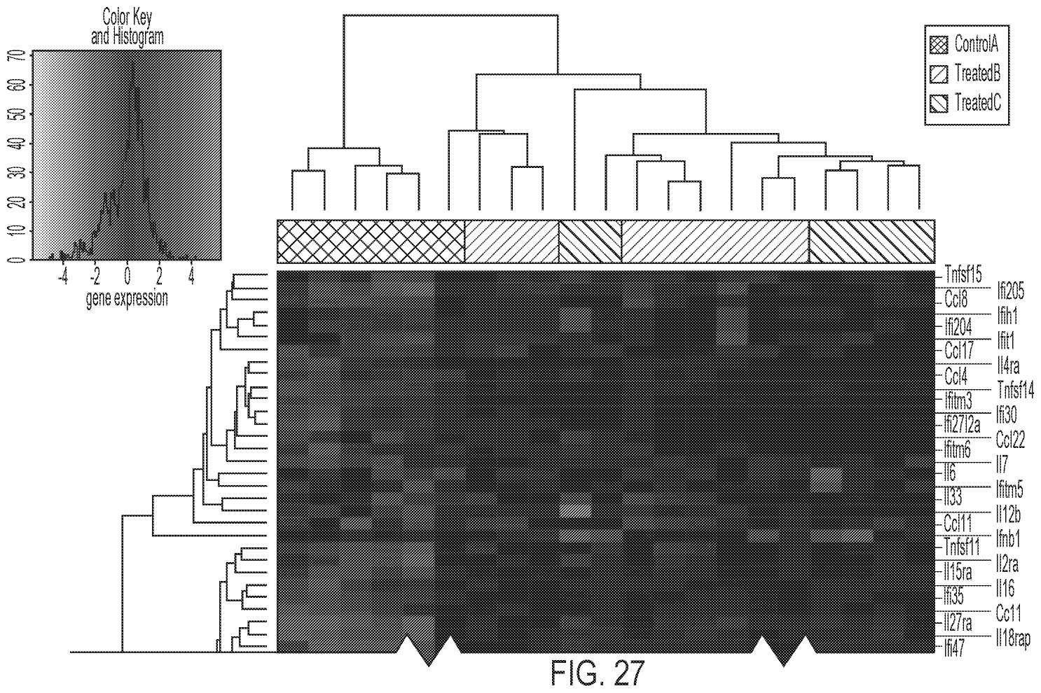

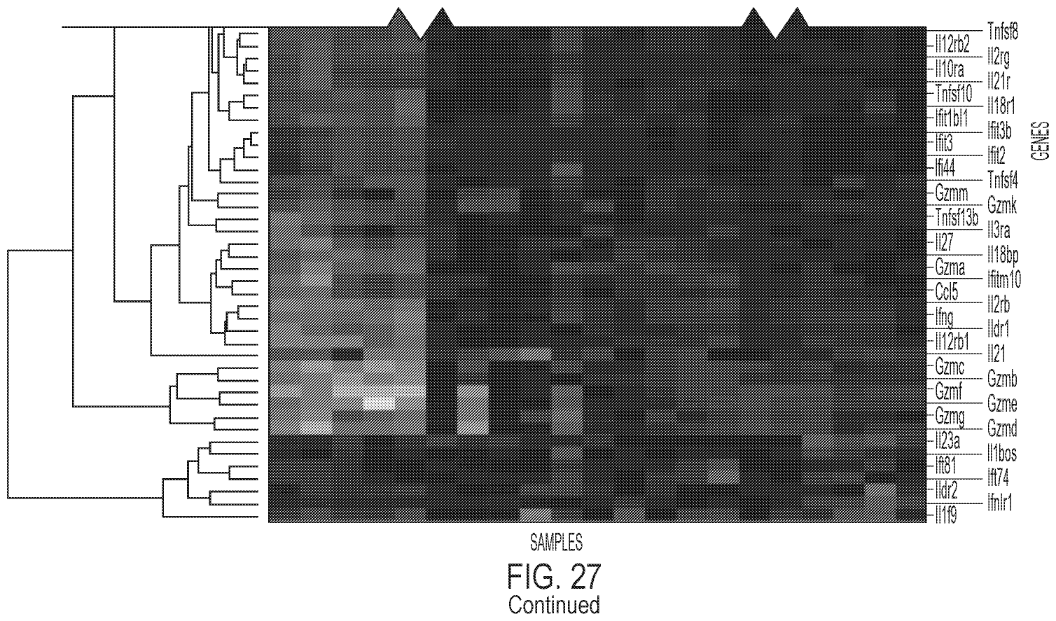

An analysis of cytokine gene expression in these three groups indicated that both the ‘hot’ and ‘cold’ αMSH-PEG-Cy5-C′ dots yield similar profiles in the TME's CD45+ immune cells versus the vehicle- treated controls (FIG. 27 and Table 2 as seen below). The expression of several granzyme genes (Gzma, Gzmb, Gzmc, Gzmd, Gzme, Gzmf, and Gzmg) was particularly robust in both ‘hot’- and ‘cold’-treated groups and ranged from 5- to 28-fold higher expression compared to the vehicle-treated group. Other inflammatory cytokines and receptors identified in this analysis include the interleukins (Il12rb1, Il18bp, Il2rb, Il27), interferon gamma (Ifng), interferon induced proteins (Ifitl, Ifit1b11, Ifit2, Ifit3, Ifit3b), tumor necrosis factor (TNF) ligand family (Tnfsf10, Tnfsf11, Tnfsf13b, Tnfsf14, Tnfsf15, Tnfsf4, Tnfsf8) and chemokine (C-C motif) ligands (Ccl1, Ccl11, Ccl17, Ccl22, Ccl4, Ccl5, Ccl8). The direct comparison of gene expression between the ‘hot’ and ‘cold’ groups does not demonstrate remarkable differences in cytokine-related expression.

TABLE 2
Mean counts cytokine expression in all samples in
Groups A, B, and C.

| | Group | Group | Group | | | |
|---|---|---|---|---|---|---|
| Genes | A | B | C | B/A¹ | C/A² | B/C³ |
| Ccl1 | 43 | 100 | 95 | 2.3 | 2.2 | 1.04 |
| Ccl11 | 34 | 98 | 47 | 2.9 | 1.4 | 2.10 |
| Ccl17 | 63 | 161 | 161 | 2.5 | 2.5 | 1.00 |
| Ccl22 | 1360 | 2804 | 2399 | 2.1 | 1.8 | 1.17 |
| Ccl4 | 2537 | 6238 | 5169 | 2.5 | 2.0 | 1.21 |
| Ccl5 | 1151 | 5541 | 4889 | 4.8 | 4.2 | 1.13 |
| Ccl8 | 3242 | 11011 | 7633 | 3.4 | 2.4 | 1.44 |
| Gzma | 243 | 1636 | 1351 | 6.7 | 5.6 | 1.21 |
| Gzmb | 1036 | 11134 | 9485 | 10.7 | 9.2 | 1.17 |
| Gzmc | 191 | 1879 | 1868 | 9.8 | 9.8 | 1.01 |
| Gzmd | 22 | 222 | 315 | 10.2 | 14.6 | 0.70 |
| Gzme | 12 | 171 | 251 | 14.2 | 20.9 | 0.68 |
| Gzmf | 24 | 424 | 674 | 17.8 | 28.3 | 0.63 |
| Gzmg | 12 | 76 | 166 | 6.2 | 13.5 | 0.46 |
| Gzmk | 135 | 339 | 596 | 2.5 | 4.4 | 0.57 |
| Gzmm | 15 | 36 | 34 | 2.4 | 2.3 | 1.06 |
| Ifi204 | 3650 | 7926 | 6411 | 2.2 | 1.8 | 1.24 |
| Ifi205 | 632 | 2616 | 1840 | 4.1 | 2.9 | 1.42 |
| Ifi2712a | 3924 | 10661 | 9479 | 2.7 | 2.4 | 1.12 |
| Ifi30 | 7050 | 15958 | 13715 | 2.3 | 1.9 | 1.16 |
| Ifi35 | 1688 | 4681 | 4708 | 2.8 | 2.8 | 0.99 |
| Ifi44 | 621 | 1708 | 1283 | 2.7 | 2.1 | 1.33 |
| Ifi47 | 2670 | 9536 | 10716 | 3.6 | 4.0 | 0.89 |
| Ifih1 | 1992 | 3996 | 2693 | 2.0 | 1.4 | 1.48 |
| Ifit1 | 874 | 2056 | 1273 | 2.4 | 1.5 | 1.62 |
| Ifit1bl1 | 292 | 1382 | 966 | 4.7 | 3.3 | 1.43 |
| Ifit2 | 4280 | 12294 | 10457 | 2.9 | 2.4 | 1.18 |
| Ifit3 | 2259 | 7610 | 5781 | 3.4 | 2.6 | 1.32 |
| Ifit3b | 506 | 1562 | 1096 | 3.1 | 2.2 | 1.43 |
| Ifitm10 | 32 | 201 | 154 | 6.4 | 4.9 | 1.30 |
| Ifitm3 | 13608 | 30597 | 29491 | 2.2 | 2.2 | 1.04 |
| Ifitm5 | 10 | 20 | 15 | 2.0 | 1.5 | 1.34 |
| Ifitm6 | 240 | 550 | 446 | 2.3 | 1.9 | 1.23 |
| Ifnb1 | 14 | 18 | 40 | 1.3 | 2.9 | 0.46 |
| Ifng | 126 | 964 | 1081 | 7.6 | 8.5 | 0.89 |
| Ifnlr1 | 97 | 55 | 31 | 0.56 | 0.32 | 1.76 |
| Ift74 | 311 | 146 | 91 | 0.47 | 0.29 | 1.60 |
| Ift81 | 343 | 148 | 123 | 0.43 | 0.36 | 1.21 |
| Il10ra | 4156 | 13381 | 10754 | 3.2 | 2.6 | 1.24 |
| Il12b | 150 | 396 | 264 | 2.6 | 1.8 | 1.50 |
| Il12rb1 | 291 | 2592 | 2509 | 8.9 | 5.6 | 1.03 |
| Il12rb2 | 196 | 834 | 657 | 4.3 | 3.4 | 1.27 |
| Il15ra | 195 | 599 | 542 | 3.1 | 2.8 | 1.10 |
| Il16 | 1154 | 2356 | 2893 | 2.0 | 2.5 | 0.81 |
| Il18bp | 562 | 3800 | 2820 | 6.8 | 5.0 | 1.35 |
| Il18r1 | 242 | 775 | 898 | 3.2 | 3.7 | 0.86 |
| Il18rap | 540 | 2089 | 2342 | 3.9 | 4.3 | 0.89 |
| Il1bos | 27 | 19 | 10 | 0.71 | 0.38 | 1.89 |
| Il1f9 | 115 | 18 | 35 | 0.16 | 0.31 | 0.51 |
| Il21 | 4 | 11 | 26 | 2.7 | 6.4 | 0.42 |
| Il21r | 2074 | 6883 | 5672 | 3.3 | 2.7 | 1.21 |
| Il23a | 46 | 36 | 10 | 0.79 | 0.23 | 3.46 |
| Il27 | 31 | 221 | 141 | 7.2 | 4.6 | 1.57 |
| Il27ra | 265 | 1122 | 1176 | 4.2 | 4.4 | 0.95 |
| Il2ra | 398 | 971 | 881 | 2.4 | 2.2 | 1.10 |
| Il2rb | 2589 | 21605 | 18374 | 8.3 | 7.1 | 1.18 |
| Il2rg | 2243 | 7383 | 7086 | 3.3 | 3.2 | 1.04 |
| Il33 | 45 | 117 | 71 | 2.6 | 1.6 | 1.66 |
| Il3ra | 468 | 1507 | 1152 | 3.2 | 2.5 | 1.31 |
| Il4ra | 8779 | 18568 | 15007 | 2.1 | 1.7 | 1.24 |
| Il6 | 192 | 452 | 236 | 2.4 | 1.2 | 1.92 |
| Il7 | 7 | 21 | 16 | 3.1 | 2.4 | 1.29 |
| Ildr1 | 40 | 273 | 279 | 6.9 | 7.0 | 0.98 |
| Ildr2 | 63 | 21 | 14 | 0.33 | 9.22 | 1.47 |
| Tnfsf10 | 387 | 1400 | 1334 | 3.6 | 3.4 | 1.05 |
| Tnfsf11 | 34 | 98 | 91 | 2.9 | 2.7 | 1.08 |
| Tnfsf13b | 16 | 75 | 77 | 4.6 | 4.7 | 0.98 |
| Tnfsf14 | 146 | 383 | 368 | 2.6 | 2.5 | 1.04 |
| Tnfsf15 | 15 | 38 | 30 | 2.5 | 2.0 | 1.26 |
| Tnfsf4 | 72 | 321 | 221 | 4.4 | 3.1 | 1.45 |
| Tnfsf8 | 93 | 374 | 305 | 4.0 | 3.3 | 1.22 |

¹The ratio of mean counts in Group B to Group A
²The ratio of mean counts in Group C to Group A
³The ratio of mean counts in Group B to Group C

Pathway enrichment analysis of differentially expressed genes with at least 4-fold change demonstrated that many of the top upregulated pathways in ‘hot’ C′ dot-treated tumors versus vehicle-treated controls are immunity, immune response, adaptive immunity, cellular response to interferons and response to virus (see Table 2 above). This analysis infers that pathways that control cytolysis, peptidase, protease, proteolysis, apoptotic response, hydrolase activity, and viral response, among others, are upregulated in the CD45+ cells that populate C′ dot-treated TME.

### Discussion

Ultrasmall silica nanoparticles with fluorescent core-shells (e.g., C′ dots and C dots) have been engineered to comprise unique combinations of biochemical features. In certain embodiments, the combination of biochemical features as presented herein allows the nanoparticles to target and treat melanoma in vivo. The alpha particle-emitting ²²⁵Ac payload allows for a potent and specific tumoricidal effect that, among other things, controls tumor growth at doses that are safe and nontoxic to normal tissue. These nanoparticles are internalized by macrophages and unexpectedly, even the unlabeled particles alone are sufficient to prompt key inflammatory immune cell changes within the tumor microenvironment. Pharmacologically, αMSH-functionalized C′ dots target melanoma, clear the host rapidly, deliver therapeutic payloads of cytotoxic alpha particles to disease and significantly alter the immune cell composition within the tumor microenvironment via macrophage processing and inflammatory signaling.

The overall pharmacokinetic profile of [²²⁵Ac]αMSH-PEG-Cy5-C′ dots is governed by the ultrasmall silica particle size and/or shape in both naive and melanoma bearing mice where the αMSH permits tumor-specific binding and internalization. Actinium-225 activity clears the blood compartment with biphasic elimination kinetics in both models. Due to the 6.0 nm diameter of these ultrasmall particles, C′ dots are readily eliminated in urine in both naive and tumor-bearing mice. Rapid renal elimination of C dots was also noted in humans and is a favorable pharmacological characteristic in translation. Specific tumor accumulation, minimal off-target tissue uptake, rapid clearance from blood, and/or facile renal elimination are make C′ dots and C dots suited for both therapeutic and diagnostic medical applications in humans.

Additional new data presented herein establishes that macrophages in naive and tumor bearing mice are also a sink for the αMSH-PEG-Cy5-C′ dots in vivo. Macrophage uptake of the silica nanoparticle is related to key changes in the immune cell profile of the TME.

Dose selection for therapeutic studies was informed from an evaluation of the maximum tolerated dose. Naive mice receiving 23.1 kBq (625 nCi) of [²²⁵Ac]αMSH-PEG-Cy5-C′ dots exhibit no toxicity (i.e., there was less than 20% weight loss and no lethargy or death at this dose level) and median survival was not reached. This absence of radiobiological effects on the health of mice is explained by the favorable pharmacokinetic characteristics of the radiolabeled αMSH-C′ dot. The radiolabeled αMSH-C′ dot does not significantly accumulate in normal tissue, and unbound drug is rapidly eliminated from the host. Higher dose levels of 225Ac-labeled C′ dots (46.3 or 92.5 kBq per mouse) were toxic and median survival was 10 days. Radiotherapeutic studies generally use about half the maximum tolerated dose (11.1 kBq, 300 nCi) to avoid non-specific effects.

Potent and specific pharmacodynamic activity was observed in a syngeneic melanoma mouse model. A single 11.1 kBq dose of [²²⁵Ac]αMSH-PEG-Cy5-C′ dot effectually controls tumor growth and improves survival compared to a nontargeted [²²⁵Ac]NH2-PEG-Cy5-C′ dot control and vehicle-treated groups. Tumor-specific [²²⁵Ac]αMSH-PEG-Cy5-C′ dot improves median survival compared to vehicle-treated mice. Specific tumor control is evidenced in the separation between the mean tumor volumes of specific and non-specific C′ dot-treated groups over the course of the study. Human dosimetry predictions for a 37 MBq dose of [²²⁵Ac]αMSH-PEG-Cy5-C′ dot project that absorbed doses to kidney, liver and lung are significantly below the dose limits for these organs.

Tumor control and immune cell changes in the TME show potent cytotoxicity and/or a dynamic, time-dependent remodeling of the immune phenotype following [²²⁵Ac]αMSH-PEG-Cy5-C′ dot treatment compared to vehicle-treated control animals. The direct pharmacological consequences of alpha particle irradiation and the silica nanoparticle contribute to tumor killing and TME remodeling. Dynamic changes in macrophage, T cell, and NK cell populations were observed over a 4-5 day period. Without wishing to be bound to any particular theory, ancillary immunotherapeutic approaches may be deployed in combination with the 225Ac-labeled C′ dot agents. RNA-seq was used to identify specific immune cell signatures in the TME that occur 4 days after treatment. Surprisingly, the ‘cold’ αMSH-PEG-Cy5-C′ dots also induced comparable changes in the TME that are similar to the ‘hot’ [225Ac]αMSH-PEG-Cy5-C′ dots. However, the ‘hot’, radiolabeled C′ dot drug was more immediately cytotoxic than the ‘cold’ C′ dot as it reduced tumor burden, thus improving overall survival. An unsupervised principal component analysis of gene expression from all samples showed overlap in both C′ dot-treated groups (i.e., labeled and unlabeled) which were distinct from the vehicle-treated controls.

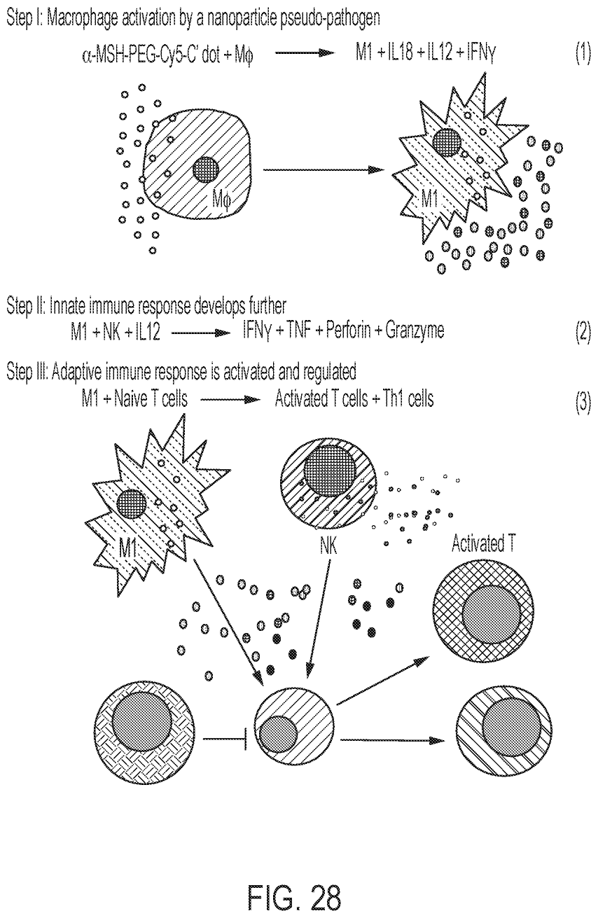

FACS analyses demonstrated that αMSH-PEG-Cy5-C′ dots were internalized by both B16-F10 melanoma and macrophages. When radiolabeled with ²²⁵Ac, the accumulation of C′ dots in tumor yields, among other things, an optimal geometry for specific cytotoxic alpha particle irradiation of the melanoma. When C′ dots are taken up by macrophages, they cue a dynamic immunoreactive environment within melanoma that engages both innate and adaptive response elements. MΦ macrophages The fraction of Treg cells also increases in the treated TME. Without wishing to be bound to any particular theory, Treg cells may suppress favorable immunotherapeutic tumor responses. αMSH-PEG-Cy5-C′ dot uptake is observed in murine IP tissue macrophages in vivo. Furthermore, human THP-1 cells (wild type and PMA-differentiated) and B16-F10 also accumulated αMSH-PEG-Cy5-C′ dots in vitro. Activated THP-1 cells are reported to express MC1-R and the data presented herein show macrophages phagocytose and accumulate αMSH-PEG-Cy5-C′ dots. Without wishing to be bound to any particular theory, the ultrasmall silica dots are phagocytosed by macrophages prompting a pseudo-pathogen immunologic response (FIG. 28). This early innate immune response subsequently engages and activates and expands the relative numbers of NK, Th1, CD8 T, and immature DC cells (see Table 3 as depicted below). Table 3 discloses the fold-changes in cytokines and cytolytic protein gene expression levels of representative immune cells found in the C′ dot activated microenvironment 96h after either treating with ‘hot’ αMSH-PEG-C′ dots or ‘cold’ αMSH-PEG-C′ dots versus vehicle-treated controls.

TABLE 3
Changes in immune cell and cytolytic protein gene expression.

| | Ratio of | Ratio of |
|---|---|---|
| | ‘hot’-to-control | ‘cold’-to-control |
| Cell type | | |
| MΦ macrophage | 0.29 | 0.11 |
| M1 macrophage | 3.1 | 2.1 |
| NK cell (activated) | 6.0 | 6.3 |
| CD8 T cell (naïve) | 2.3 | 2.6 |
| CD8 T cell (activated) | 3.3 | 3.3 |
| Th1 cell | 6.2 | 3.0 |
| Regulatory T cell | 3.0 | 4.1 |
| Dendritic cell (immature) | >>2 | >>5 |
| Cytokines and | | |
| cytolytic proteins | | |
| IL18 | 6.8 | 5.0 |
| IL12 | 8.9 | 8.6 |
| IFNγ | 7.6 | 8.5 |
| TNF | 3.6 | 3.4 |
| Perforin | ND | ND |
| Granzyme | 17.8 | 28.3 |

Upregulated cytokine and cytolytic protein gene expression is additional evidence that numerous key inflammatory signals increase in the TME as a consequence of C′ dot-macrophage pharmacology (Table 3). Furthermore, the expression of granzymes, interleukins, interferon gamma, interferon induced proteins, TNF ligands, and chemokines describe a complex milieu of inflammatory signaling molecules arising from the C′ dot component of the drug. Upregulated pathways in [²²⁵Ac]αMSH-PEG-Cy5-C′ dot-treated tumors versus vehicle-treated control are immunity, immune response, adaptive immunity, and cellular response to interferons and are consistent with response to a viral pathogen.

The C′ dot component of the drug prompts inflammatory changes in the TME and is an immunotherapeutic approach to eradicating residual disease. Furthermore, as the C′ dots are a synthetic nanoscale particle and not a live pathogen, the initial phenotype response has a finite lifetime in vivo and is not a self-sustaining event. Without wishing to be bound to any particular theory, the observed increase in TME Tregs dampens the tumoricidal immunologic activity. The fraction of suppressive regulatory T cells in the TME increases in both the ‘hot’ and ‘cold’ targeted C′ dot-treated groups, increasing several-fold over baseline values in vehicle-treated tumor. In certain embodiments, the C′ dots are used with anti-PD1 or anti-CTLA-4 checkpoint blockade strategies. In certain embodiments, the CD47-SIRPα signaling axes in macrophages is exploited to improve long-term tumor control. In other certain embodiments, tumor killing from activated NK cells is intensified with the introduction of IL12 or IFN gamma. In certain embodiments, a method entails administering only the ‘cold’ C′ dots to sustain the pseudo-pathogenic response.

### Conclusions

In certain embodiments, second generation ultrasmall fluorescent core-shell silica nanoparticles (e.g., C′ dots) can target melanoma in vivo via covalently attached αMSH peptide moieties. In certain embodiments, modified C′ dots produce potent and/or specific cytotoxicity due to a ²²⁵Ac payload. In certain embodiments, agents comprising C′ dots are colloidally stable in aqueous solutions, biocompatible, and/or exhibits a narrow size distribution. In certain embodiments, a therapeutic alpha particle payload conjugated to a C′ dot imparts cytotoxic high linear energy therapy.

Surprisingly, both radiolabeled [225Ac]αMSH-PEG-Cy5-C′ (‘hot’) and unlabeled αMSH-PEG-Cy5-C′ dots (‘cold’) similarly cue significant changes in the TME immune cell signatures. Without wishing to be bound to a particular theory, the inflammatory TME results from a pseudo-pathogenic response of macrophages to the C′ dot. This immune response upregulates the fraction of M1 macrophages, Th1, monocytes, activated NK, and immature DC cells in TME. Inflammatory pathways are engaged by this immune cell composite yielding a cytokine milieu that provides a distinctive opportunity to augment alpha therapy with ancillary immunotherapeutic approaches moving forward.

### Pharmaceutically Acceptable Compositions

According to another embodiment, the invention provides a composition comprising a nanoparticle as described herein and a pharmaceutically acceptable carrier, adjuvant, or vehicle. The amount of nanoparticle in administered compositions presented herein is such that is effective to measurably induce changes in immune cells of the tumor microenvironment, in a biological sample or in a patient. In certain embodiments, the amount of nanoparticle in administered compositions is such that is effective to measurably induce changes in immune cells of the tumor microenvironment, in a biological sample or in a patient. In certain embodiments, a composition described herein is formulated for administration to a patient in need of such composition. In some embodiments, a composition is formulated for oral administration to a patient.

The term “patient,” as used herein, means an animal, preferably a mammal, and most preferably a human. In certain embodiments, the patient is a mouse.

The term “pharmaceutically acceptable carrier, adjuvant, or vehicle” refers to a non-toxic carrier, adjuvant, or vehicle that does not destroy the pharmacological activity of the nanoparticle with which it is formulated. Pharmaceutically acceptable carriers, adjuvants or vehicles that may be used in the compositions of various embodiments of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of certain embodiments of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of certain embodiments of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of certain embodiments described herein may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of certain embodiments described herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of certain embodiments described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of embodiments described herein include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of certain embodiments of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of certain embodiments described herein are formulated for oral administration. Such formulations may be administered with or without food. In some embodiments, pharmaceutically acceptable compositions of certain embodiments described herein are administered without food. In other embodiments, pharmaceutically acceptable compositions of certain embodiments described herein are administered with food.

The amount of nanoparticles of certain embodiments described herein that may be combined with the carrier materials to produce a composition in a single dosage form will vary depending upon the host treated, the particular mode of administration. In certain embodiments, a dosage may be prepared to have a concentration of up to 100 μM of nanoparticles (e.g., up to 80μM of nanoparticles). In certain embodiments, multiple dosage may be administered multiple times as part of a treatment regimen.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a particular component in the composition may also depend upon the particular nanoparticle in the composition.

What is claimed is:

1. A method of treatment of a subject (e.g., a subject having been diagnosed with cancer), the method comprising administering a composition comprising ultrasmall (e.g., no greater than 20 nm in diameter, e.g., no greater than 10 nm in diameter) nanoparticles (e.g., a silica-containing, e.g., silica-based nanoparticle) to activate a tumor microenvironment (e.g., macrophages, T cells, and/or antigen-presenting cells (APCs, such as dendritic cells)).

2. The method of claim 1, comprising administering the composition comprising ultrasmall nanoparticles in concert with, or as part of, checkpoint inhibitor therapy (e.g., anti-PD1), or radiotherapy, or a combination of both radiotherapy and checkpoint inhibitor therapy.

3. The method of any one of the preceding claims, wherein the nanoparticle comprises a radiolabel (e.g., ²²⁵Actinium).

4. The method of any one of the preceding claims, wherein the nanoparticle comprises 1 to 25 targeting ligands (e.g., 2 to 20 ligands, 5 to 15 ligands, 5 to 10 ligands, or about 6-8 ligands).

5. The method of claim 4, wherein the targeting ligand is a targeting ligand for a cellular receptor (e.g., MC1-R, PSMA, etc.).

6. The method of claim 4 or 5, wherein the targeting ligand comprises αMSH.

7. The method of any one of the preceding claims, wherein the nanoparticle comprises a heterogeneous surface characterized by one or more of (i) to (iv) as follows: (i) an unincorporated dye; (ii) variation in a PEG coating (e.g., due to length of PEG chains and/or number of PEG chains per nanoparticle, e.g., said number from about 100 to about 500 chains per nanoparticle); (iii) variation in dye encapsulation (e.g., by PEG); and (iv) number of targeting ligands.

8. The method of any one of the preceding claims, wherein the nanoparticle has a hydrodynamic diameter no greater than 10 nm (e.g., wherein the hydrodynamic diameter is in a range from 1 nm to 10 nm).

9. The method of any one of the preceding claims, wherein the nanoparticle comprises a silica core.

10. The method of claim 9, wherein the silica core has a diameter less than 10 nm (e.g., less than 9 nm, e.g., less than 8 nm, e.g., less than 7 nm, e.g., less than 6 nm, e.g., within a range from 2.7 nm to 5.8 nm).

11. The method of any one of the preceding claims, wherein the nanoparticle comprises a polyethylene glycol (PEG) shell.

12. The method of claim 11, wherein the thickness of the PEG shell is less than 2 nm (e.g., about 1 nm).

13. The method of any one of the preceding claims, wherein the nanoparticles have a silica composition such that ferroptosis is not induced (e.g., ferroptosis is switched “off”).

14. The method of claim 13, wherein the nanoparticles are made using a ratio of phosphonate-silane to tetramethyl orthosilicate (TMOS) in a reaction feed at or above 20%.

15. The method of any one of the preceding claims, wherein the nanoparticles have a silica composition such that ferroptosis may be induced (e.g., ferroptosis is not switched “off”).

16. The method of claim 15, wherein the nanoparticles are made using a ratio of phosphonate-silane to tetramethyl orthosilicate (TMOS) in a reaction feed in a range from about 0% to about 20%.

17. The method of any one of the preceding claims, wherein the nanoparticle comprises a chelator.

18. The method of claim 17, wherein the chelator is selected from the group comprising DOTA-Bz-SCN, 1,4,7,10-tetraazacyclododecane-1,4,7,10-tetraacetic acid (DOTA), and desferoxamine (DFO).

19. The method of any one of the preceding claims, wherein the nanoparticle is non-toxic to normal tissue.

20. The method of any one of the preceding claims, wherein the nanoparticles are internalized (e.g., phagocytosed) within one or more cell types (e.g., macrophages, tumor cells, THP-1 cells) of the microenvironment.

21. The method of claim 20, wherein the one or more cell types comprise macrophages, cancer cells, and/or THP-1 cells.

22. The method of any one of the preceding claims, wherein the tumor is a cancer.

23. The method of claims 22, wherein the cancer is a glioma.

24. The method of claim 22, wherein the cancer is melanoma.

25. The method of any one of the preceding claims, wherein local concentration of nanoparticles within the microenvironment of the tumor is in a range from about 0.013 nmol/cm³ to about 86 nmol/cm³ or from about 0.013 nmol/cm³ to about 0.14 nmol/cm3 or from about 8 nmol/cm³ to about 86 nmol/cm³ (e.g., wherein an administered dose (e.g., by IV) has particle concentration from about 100 nM to about 60 μM, or wherein an administered dose has particle concentration less than 150 nM (e.g., less than 100 nM, e.g., less than 50 nM, less than 10 nM, less than 5 nM).

26. The method of any one of the preceding claims, wherein the activation of the microenvironment of the tumor comprises a change (e.g., an increase) in at least one M1 macrophage polarization marker.

27. The method of claim 26, wherein the at least one M1 macrophage polarization marker is a member selected from the group consisting of iNOS, TNFα, IL12p70, IL12p40, CD86, and CD8.

28. The method of any one of the preceding claims, wherein the activation of the microenvironment of the tumor comprises a change (e.g., a decrease) in at least one M2 macrophage polarization marker.

29. The method of claim 28, wherein the at least one M2 macrophage polarization marker is a member selected from the group consisting of IL-4, IL-10, and IL-13.

30. The method of any one of the preceding claims, wherein the activation of the tumor microenvironment causes a change (e.g., an increase) in one or more cytokines and/or cytolytic proteins.

31. The method of claim 30, wherein the one or more cytokines and/or cytolytic proteins comprises at least one member selected from the group consisting of IL18, IL12, IFN gamma, TNF, and a Granzyme.

32. The method of any one of claims 2 to 28, wherein the activation of the microenvironment comprises changing (e.g., increasing, decreasing) a population and/or level of activation of one or more cell types within the microenvironment.

33. The method of claim 32, wherein the method comprises increasing the population and/or level of activation of one or more immune-related cell types.

34. The method of claim 33, wherein the one or more immune-related cell types comprise at least one member selected from the group consisting of immature dendritic cells, regulatory T cells, monocytes, M1 macrophages, and natural killer cells.

35. The method of claim 32, wherein the method comprises decreasing the population and/or level of activation of one or more immune-related cell types.

36. The method of claim 35, wherein the one or more immune-related cell types comprise M2 macrophages and/or MΦ macrophages.

37. The method of any one of the preceding claims, wherein the composition is administered in multiple doses (e.g., at fixed intervals, e.g., every 1, 2, 3, 5, or 10 days).

38. The method of any one of the preceding claims, wherein the method comprises administering a macromolecule (e.g., a protein).

39. The method of claim 38, wherein the macromolecule is an interleukin (e.g., IL12).

40. The method of claim 38, wherein the macromolecule is an interferon (e.g., IFN gamma).

41. The method of any one of the preceding claims, wherein the method comprises activating the tumor microenvironment in the absence of ferroptosis.

42. The method of any one of the preceding claims, wherein the method comprises administering one or more regulators of ferroptosis.

43. The method of claim 42, wherein the regulator of ferroptosis is an inhibitor of ferroptosis.

44. The method of claim 43, wherein the one or more inhibitors of ferroptosis comprises a member selected from the group consisting of liproxstatin-1, ferrostatin-1, and/or other compounds which scavenge lipid peroxides.

45. A composition for use in the method of any one of the preceding claims, the composition comprising ultrasmall nanoparticles having the following attributes:
(i) a number of targeting ligands (e.g., αMSH) from 5 to 15 per nanoparticle;
(ii) a heterogeneous surface characterized by one or more of (a) to (d) as follows: (a) an unincorporated dye; (b) a variation in a PEG coating (e.g., due to length of PEG chains and/or number of PEG chains per nanoparticle, e.g., said number from about 100 to about 500 chains per nanoparticle); (c) a variation in dye encapsulation (e.g., by PEG); and (d) a number of targeting ligands (e.g., from 1 to 60 per nanoparticle, or from 1 to 15 per nanoparticle, or from 40 to 60 per nanoparticle);
(iii) a particle core and shell having a hydrodynamic diameter in a range from 4.7 nm to 7.8 nm (e.g., with a silica core diameter in a range from 2.7 nm to 5.8 nm and/or with a PEG shell thickness of about 1 nm); and
(iv) a silica composition controlled for ferroptosis “switch-off” (e.g., wherein the nanoparticles are made using a ratio of phosphonate-silane to tetramethyl orthosilicate (TMOS) in a reaction feed at or above 20% such that ferroptosis may occur, or wherein the nanoparticles are made using a ratio of phosphonate-silane to tetramethyl orthosilicate (TMOS) in a reaction feed from 0% to 20% such that ferroptosis may not occur.

46. A composition (e.g., a pharmaceutical composition) for use in a medicament, the composition comprising ultrasmall nanoparticles having the following attributes:
(i) a number of targeting ligands (e.g., αMSH) from 5 to 15 per nanoparticle;
(ii) a heterogeneous surface characterized by one or more of (a) to (d) as follows: (a) an unincorporated dye; (b) a variation in a PEG coating (e.g., due to length of PEG chains and/or number of PEG chains per nanoparticle, e.g., said number from about 100 to about 500 chains per nanoparticle); (c) a variation in dye encapsulation (e.g., by PEG); and (d) a number of targeting ligands (e.g., from 1 to 60 per nanoparticle, or from 1 to 15 per nanoparticle, or from 40 to 60 per nanoparticle);
(iii) a particle core and shell having a hydrodynamic diameter in a range from 4.7 nm to 7.8 nm (e.g., with a silica core diameter in a range from 2.7 nm to 5.8 nm and/or with a PEG shell thickness of about 1 nm); and
(iv) a silica composition controlled for ferroptosis “switch-off” (e.g., wherein the nanoparticles are made using a ratio of phosphonate-silane to tetramethyl orthosilicate (TMOS) in a reaction feed at or above 20% such that ferroptosis may occur, or wherein the nanoparticles are made using a ratio of phosphonate-silane to tetramethyl orthosilicate (TMOS) in a reaction feed from 0% to 20% such that ferroptosis may not occur.

47. A treatment comprising a therapeutically effective amount of a composition (e.g., wherein the composition comprises a tumor microenvironment activating nanoparticle with a ligand for targeting MC1-R) for use in a method of treating cancer in a subject.

48. A method of treating cancer in a subject, the method comprising:
administering a composition to the subject to activate a tumor microenvironment.

49. The method of claim 48, wherein the composition comprises a nanoparticle.

50. The method of any one of claims 1 to 44, wherein the nanoparticle does not comprise a targeting ligand.

51. The method of claim 50, wherein the nanoparticle comprises PEG (e.g., a PEG coating).
